(19) United States
(12) Patent Application Publication
(10) Pub. No.: US 2009/0155254 A1
(43) Pub. Date: Jun. 18, 2009

(54) Affinity Regions

(76) Inventors: Martijn Frans Ben Gerard Gebbink, Eemnes (NL); Barend Bouma, Houten (NL)

Correspondence Address: TRASKBRITT, P.C., P.O. BOX 2550, SALT LAKE CITY, UT, 84110, US

(21) Appl. No.: 12/224,087

(22) PCT Filed: Feb. 16, 2007
(86) PCT No.: PCT/NL2007/050063

§ 371 Date: Feb. 18, 2009

(30) Foreign Application Priority Data
Feb. 16, 2006 (EP) 06075355.5

(51) Int. Cl. A61K 39/395; G01N 33/566; C07K 16/18; G01N 33/53

(52) U.S. Cl. 424/133.1; 436/501; 530/387.1; 424/130.1; 435/7.1; 435/7.2

(57) ABSTRACT

The present invention provides a method for selecting from a collection of IgIV molecules, at least one IgIV molecule comprising an affinity region that is capable of interacting with a misfolded protein and/or with an epitope of a cross-β structure and/or with an epitope of a protein comprising a cross-β structure, said method comprising contacting a collection of IgIV molecules with a misfolded protein and/or with a cross-β structure and/or with a protein comprising a cross-β structure and collecting at least one IgIV molecule comprising an affinity region interacting with said misfolded protein and/or epitope.

The invention relates to the fields of biochemistry, molecular biology, structural biology and medicine.

Immune Globulin Intravenous (IgIV) are immunoglobulins of apparently healthy animals or humans which immunoglobulins are collected from serum or blood. IgIV are prescribed to animals and humans that have a lack of antibodies. The cause of said lack of antibodies may be an illness affecting the immune system, such as for example AIDS, or an inborn error causing complete or partial a-gammaglobulinaemia or hypogammaglobulinaemia such as for example a primary immunodeficiency syndrome or severe combined immunodeficiency syndrome (SCIDS). Immunoglobulins collected from human serum or blood are commercially available under many different names such as for example “intravenous human immunoglobulins” (IgIV, or IVIg). Since IgIV were first introduced in 1981 for immunoglobulin substitution therapy of above-described immunodeficient humans, they have also been applied off label to patients suffering of a wide variety of diseases and in a number of cases, experimental treatment with IgIV turned out to be successful.

Because the mechanism of action has not been elucidated up to now, many of the off label treatments with IgIV were trial and error experiments, and the outcome was rather unpredictable.

The treatment with IgIV is not without risks. Since 1981, the Food and Drug Administration (FDA) has received over 114 worldwide (approximately 83 U.S.) adverse event reports of renal dysfunction and/or acute renal failure associated with the administration of these products. Although acute renal failure was successfully managed in the majority of cases, deaths were reported in 17 patients worldwide. Many of the patients who died had serious underlying conditions. For further reported side effects related to administering of IgIV in patients see Table 1.

In conclusion, the treatment of humans with IgIV is not clearly limited to a specific disease condition, a clear understanding of the mode of action is lacking and the outcome of an IgIV treatment for a new disease entity is unpredictable. The administration of large amounts of IgIV involves the risk of adverse side effects. Therefore, there is a need for improvement of current IgIV treatment.

It is an object of the present invention to provide means and methods for improving IgIV therapy. It is a further object to provide a selection and/or purification of a group of immunoglobulins from an IgIV pool, and an alternative mode of preparation of immunoglobulins or a functional equivalent thereof in order to obtain an immunoglobulin or equivalent thereof, suitable for treatment of a disease or a group of diseases, with at least one improved property as compared to IgIV, such as for instance a reduced risk of adverse side effects and/or an improved therapeutic action. Up to now, a person skilled in the art would not know how to start and which selection to make.

The present invention provides the insight that a selection of IgIV that is enriched in IgIV molecules capable of interacting with an epitope of a misfolded protein, an epitope of a cross-β structure and/or with an epitope of a protein comprising a cross-β structure has improved properties as compared to currently used IgIV. Said selection is preferred over currently used IgIV, amongst other things because adverse side effects are at least in part prevented and/or therapeutic action is improved.

A misfolded protein is defined herein as a protein with a structure other than a native, non-amyloid, non-cross-β structure. Hence, a misfolded protein is a protein having a non-native three dimensional structure, and/or a cross-β structure, and/or an amyloid structure. Protein misfolding is of etiological importance to a large number of diseases, often related to aging (such as amyloid diseases). Misfolding diseases are also referred to as conformational diseases. At present over 30 misfolding diseases, including but not limited to localized and systemic amyloidoses, like Alzheimer's disease and dialysis related amyloidosis, Parkinson's disease, and Huntington's diseases, have been described as such. We previously disclosed that, in addition to these known misfolding diseases, many other diseases, of which a number with still partly or largely unknown etiology, including (auto-)immune diseases and atherosclerosis, are associated with protein misfolding (patent WO 2004 004698 (EP1536778) and related patents). For many of these diseases no adequate treatment or cure is available. We also disclosed that other processes, of which several can be disease related, such as clearance from the body of obsolete proteins at the end of their life-time, blood coagulation, platelet aggregation and fibrinolysis, are associated with protein misfolding.

Besides the role of misfolded proteins in disease initiation and/or disease progression, protein misfolding also underlies complications, such as adverse generation of auto-antibodies, anaphylactic responses and other inflammatory or allergic reactions, associated with the use of protein pharmaceuticals. For this reason protein misfolding is of major concern during production, storage and use of protein-based drugs.

Finally, misfolded proteins contribute to induction of immunity, and misfolded proteins can be used to trigger and/or potentiate an immune response, for example for the use in vaccines.

Misfolded proteins tend to multimerize and can initiate fibrillization. This can result in the formation of amorphous aggregates that can vary greatly in size. In certain cases misfolded proteins are more regular and fibrillar in nature. The term amyloid has initially been introduced to define the fibrils, which are formed from misfolded proteins, and which are found in organs and tissues of patients with the various known misfolding diseases, collectively termed amyloidoses. Commonly, amyloid appears as fibrils with indefinite length and with a mean diameter of 10 nm, is deposited extracellularly, stains with the dyes Congo red and Thioflavin T (ThT), shows characteristic green birefringence under polarized light when Congo red is bound, comprises β-sheet secondary structure, and contains the characteristic crossbeta conformation (see below) as determined by X-ray fibre diffraction analysis. However, since it has been determined that protein misfolding is a more general phenomenon and since many characteristics of misfolded proteins are shared with amyloid, the term amyloid has been used in a broader scope. Now, the term amyloid is also used to define intracellular fibrils and fibrils formed in vitro. Also the terms amyloid-like and amylog are used to indicate misfolded proteins with properties shared with amyloids, but that do not fulfill all criteria for amyloid, as listed above.

In conclusion, misfolded proteins are highly heterogeneous in nature, ranging from monomeric misfolded proteins, to small oligomeric species, sometimes referred to as protofibrils, larger aggregates with amorphous appearance, up to large highly ordered fibrils, all of which appearances can share structural features reminiscent to amyloid. As used herein, the term “misfoldome” encompasses any collection of misfolded proteins.

Amyloid and misfolded proteins that do not fulfill all criteria for being identified as amyloid can share structural and functional features with amyloid and/or with other misfolded proteins. These common features are shared among various misfolded proteins, independent of their varying amino acid sequences. Shared structural features include for example the binding to certain dyes, such as Congo red, ThT, Thioflavin S, accompanied by enhanced fluorescence of the dyes, multimerization, and the binding to certain proteins, such as tissue-type plasminogen activator (tPA), the receptor for advanced glycation end-products (RAGE) and chaperones, such as heat shock proteins, like BiP (grp78 or immunoglobulin heavy chain binding protein). Shared functional activities include the activation of tPA and the induction of cellular responses, such as inflammatory responses, and induction of cell toxicity.

A unique hallmark of a subset of misfolded proteins such as for instance amyloid is the presence of the crossbeta conformation or a precursor form of the crossbeta conformation.

A cross-β structure is a secondary structural element in peptides and proteins. A cross-β structure (also referred to as a “cross-β”, a “cross beta” or a “crossbeta” structure”) is defined as a part of a protein or peptide, or a part of an assembly of peptides and/or proteins, which comprises single β-strands (stage 1) and/or a(n ordered) group of β-strands (stage 2), and/or typically a group of β-strands arranged in a β-sheet (stage 3), and/or in particular a group of stacked β-sheets (stage 4), also referred to as “amyloid”. A crossbeta structure is formed following formation of a crossbeta structure precursor form upon protein misfolding like for example denaturation, proteolysis or unfolding of proteins. A crossbeta structure precursor is defined as any protein conformation that precedes the formation of any of the aforementioned structural stages of a crossbeta structure. These structural elements present in crossbeta structure (precursor) are typically absent in globular regions of (native parts of) proteins. The presence of crossbeta structure is for example demonstrated with X-ray fibre diffraction or binding of Thioflavin T or binding of Congo red, accompanied by enhanced fluorescence of the dyes.

A typical form of a crossbeta structure precursor is a partially or completely misfolded protein. A typical form of a misfolded protein is a partially or completely unfolded protein, a partially refolded protein, a partially or completely aggregated protein, an oligomerized or multimerized protein, or a partially or completely denatured protein. A crossbeta structure or a crossbeta structure precursor can appear as monomeric molecules, dimeric, trimeric, up till oligomeric assemblies of molecules, and can appear as multimeric structures and/or assemblies of molecules.

Crossbeta structure (precursor) in any of the aforementioned states can appear in soluble form in aqueous solutions and/or organic solvents and/or any other solutions. Crossbeta structure (precursor) can also be present as solid state material in solutions, like for example as insoluble aggregates, fibrils, particles, like for example as a suspension or separated in a solid crossbeta structure phase and a solvent phase.

Protein misfolding, formation of crossbeta structure precursor, formation of aggregates or multimers and/or crossbeta structure can occur in any composition comprising peptides, of at least 2 amino acids, and/or protein(s). The term “peptide” is intended to include oligopeptides as well as polypeptides, and the term “protein” includes proteinaceous molecules including peptides, with and without post-translational modifications such as glycosylation and glycation. It also includes lipoproteins and complexes comprising a proteinaceous part, such as protein-nucleic acid complexes (RNA and/or DNA), membrane-protein complexes, etc. As used herein, the term “protein” also encompasses proteinaceous molecules, peptides, oligopeptides and polypeptides. Hence, the use of “protein” or “protein and/or peptide” in this application have the same meaning.

A typical form of stacked β-sheets is in a fibril-like structure in which the β-sheets are stacked in either the direction of the axis of the fibril or perpendicular to the direction of the axis of the fibril. The direction of the stacking of the β-sheets in cross-β structures is perpendicular to the long fiber axis. A cross-β structure conformation is a signal that triggers a cascade of events that induces clearance and breakdown of the obsolete protein or peptide. When clearance is inadequate, unwanted proteins and/or peptides aggregate and form toxic structures ranging from soluble oligomers up to precipitating fibrils and amorphous plaques. Such cross-β structure conformation comprising aggregates underlie various diseases, such as for instance, Huntington's disease, amyloidosis type disease, atherosclerosis, diabetes, bleeding, thrombosis, cancer, sepsis and other inflammatory diseases, rheumatoid arthritis, transmissible spongiform encephalopathies such as Creutzfeldt-Jakob disease, Multiple Sclerosis, auto-immune diseases, diseases associated with loss of memory such as Alzheimer's disease, Parkinson's disease and other neuronal diseases (epilepsy), encephalopathy and systemic amyloidoses.

A cross-β structure is for instance formed during unfolding and refolding of proteins and peptides. Unfolding of peptides and proteins occur regularly within an organism. For instance, peptides and proteins often unfold and refold spontaneously at the end of their life cycle. Moreover, unfolding and/or refolding is induced by environmental factors such as for instance pH, glycation, oxidative stress, heat, irradiation, mechanical stress, proteolysis and so on. As used herein, the term “cross-β structure” also encompasses any crossbeta structure precursor and any misfolded protein, even though a misfolded protein does not necessarily comprise a crossbeta structure. The term “crossbeta binding molecule” or “molecule capable of specifically binding a crossbeta structure” also encompasses a molecule capable of specifically binding any misfolded protein.

The terms unfolding, refolding and misfolding relate to the three-dimensional structure of a protein or peptide. Unfolding means that a protein or peptide loses at least part of its three-dimensional structure. The term refolding relates to the coiling back into some kind of three-dimensional structure. By refolding, a protein or peptide can regain its native configuration, or an incorrect refolding can occur. The term “incorrect refolding” refers to a situation when a three-dimensional structure other than a native configuration is formed. Incorrect refolding is also called misfolding. Unfolding and refolding of proteins and peptides involves the risk of cross-β structure formation. Formation of cross-β structures sometimes also occurs directly after protein synthesis, without a correctly folded protein intermediate.

### Crossbeta Pathway: Response to Misfolded Proteins

We previously disclosed a biological mechanism that senses occurrence of misfolded proteins, resulting in breakdown and clearance of the misfolded proteins, termed the Crossbeta Pathway (patent WO 2004 004698). We experimentally identified a number of proteins, including tPA and the closely related proteins factor XII, hepatocyte growth factor activator (HGFA) and fibronectin, that recognize misfolded proteins, with structural features common to proteins comprising crossbeta structure or a crossbeta structure precursor form. We also disclosed that, based on analysis of the literature, a number of additional proteins, including cell surface receptors, are implicated in the response of the body to misfolded proteins, including clearance of misfolded proteins, and thus are part of the Crossbeta Pathway. We disclosed that a number of these proteins, like tPA and its relatives, are able to recognize misfolded proteins directly. Said proteins were known to bind a large number of ligands that seemed unrelated with respect to 3D structure and/or amino-acid sequence. These protein ligands are often implicated in diseases, but the presence of a common structural or sequential mode of recognition was not identified earlier. Collectively, tPA, its relatives and other proteins that recognize misfolded proteins are thus part of mechanisms that facilitate the clearance of misfolded proteins, i.e. the Crossbeta Pathway. Examples of physiological processes in which the Crossbeta Pathway is involved are long term potentiation, innate immunity, adaptive immunity, angiogenesis, blood coagulation, thrombus formation and fibrinolysis. Malfunctioning of the Crossbeta Pathway will result in proteins that form dangerous misfolded proteins, either or not accompanied by structural features commonly seen in amyloid, like for example aggregates or fibrils with crossbeta conformation. As stated above and before in patent application WO 2004 004698, misfolded proteins underlie various health problems and diseases, some of which are previously associated with protein misfolding and others that have not yet been associated as such. These health problems and diseases include Huntington's disease, localized amyloidoses, atherosclerosis, diabetes, bleeding, thrombosis, cancer, sepsis, inflammatory diseases, rheumatoid arthritis (RA), multiple sclerosis (MS), other auto-immune diseases, diseases associated with loss of memory such as Alzheimer's disease (AD), Parkinson's disease and other neuronal diseases like for example epilepsy, encephalopathy, encephalitis, cataract, systemic amyloidoses, transmissible spongiform encephalopathies, such as Creutzfeldt-Jakob disease, and amyloidosis related to dialysis with patients suffering from renal insufficiency.

In conclusion, the Crossbeta Pathway comprises molecules, some of which directly bind misfolded proteins, termed crossbeta structure binding compounds or crossbeta binding compounds or misfolded protein binding compounds, which contribute to the sensing, the breakdown and/or the clearance of misfolded proteins. The Crossbeta Pathway senses any non-native 3D fold of a protein and responds by means of various modes. The Crossbeta Pathway also comprises molecules, such as chaperones, that are able to interact with misfolded proteins in order to assist in folding and/or refolding, in order to prevent accumulation of aggregates, fibrils, and/or precipitates of misfolded proteins.

For example, tPA is a serine protease that is activated in response to direct binding to misfolded proteins. One such misfolded protein is fibrin, present in a blood clot. Upon activation, tPA generates plasmin from the zymogen plasminogen. The serine protease plasmin in turn cleaves many substrates, such as proenzymes, like procollagenases, as well as extracellular matrix proteins, like fibrin. As such tPA initiates a cascade of events to degrade aggregates of misfolded proteins, such as blood clots.

Another example is RAGE. This receptor is involved in binding glycated proteins, amyloid and other ligands, that comprise amyloid properties, and is implicated in the pathology of many diseases, such as amyloidosis, diabetes and auto-immune diseases. Administration of a soluble form of this receptor has beneficial effects in animal models of several of the aforementioned protein misfolding diseases.

Yet another example of misfolded protein binding molecules that are involved in the Crossbeta Pathway are the chaperones, or heat shock proteins (HSPs), or stress proteins. The fact that chaperones like for example haptoglobin and clusterin, assist in prevention of formation of aggregates of misfolded proteins in an ATP independent manner make them candidates to play an important role in the Crossbeta Pathway. It is likely that a series of proteins that sample protein conformation act in concert. Amongst these proteins that act in concert in the Crossbeta Pathway are chaperones, like for example HSP60, HSP90, DNAK, clusterin, haptoglobin, gp96, BiP, other (extracellularly located) HSPs, proteases, like for example HGFA, tPA, plasminogen, factor XII, IVIg, and cell surface receptors. Cell surface receptors implicated in the Crossbeta Pathway include low density lipoprotein receptor related protein (LRP, CD91) and relatives, CD36, scavenger receptor A, scavenger receptor B-I, RAGE, collectively also referred to in literature as multiligand receptors.

In summary, the Crossbeta Pathway is capable of preventing misfolded proteins to form toxic structures like for example amyloid crossbeta structure oligomers and fibrils, and is capable of degrading and clearance of (aggregates of) misfolded proteins. As part of the Crossbeta Pathway misfolded proteins bind to multiligand misfolded protein binding receptors, resulting in endocytosis and subsequent proteolytic breakdown.

Hence, modulation of the Crossbeta Pathway provides treatment opportunities for protein misfolding diseases.

### Misfolding Diseases

As mentioned above, diseases associated with protein misfolding, termed protein misfolding diseases, misfolded protein diseases, protein misfolding disorder, conformational diseases, misfolded protein related and/or associated diseases, or protein folding disorders, include amyloidoses, and protein misfolding is also associated with many other diseases and health problems and physiological processes, not necessarily defined by the term amyloidosis or protein misfolding disorder, of which several are mentioned above.

According to the present invention, a selection of IgIV that is enriched in IgIV molecules capable of interacting with a misfolded protein and/or with an epitope of a cross-β structure and/or with an epitope of a protein comprising a cross-β structure has at least one improved property as compared to currently used IgIV. The invention therefore provides a method for selecting from a collection of IgIV molecules at least one IgIV molecule capable of interacting with a misfolded protein and/or with an epitope of a cross-β structure and/or with an epitope of a protein comprising a cross-β structure. This is preferably performed by contacting a collection of IgIV molecules with a misfolded protein and/or with a cross-β structure and/or with a protein comprising a cross-β structure and collecting at least one IgIV molecule comprising an affinity region interacting with said protein and/or epitope. Provided is therefore a method for selecting from a collection of IgIV molecules, at least one IgIV molecule comprising an affinity region that is capable of interacting with a misfolded protein and/or with an epitope of a cross-β structure and/or with an epitope of a protein comprising a cross-β structure, said method comprising contacting a collection of IgIV molecules with a misfolded protein and/or with a cross-β structure and/or a protein comprising a cross-β structure and collecting at least one IgIV molecule comprising an affinity region interacting with said epitope.

An affinity region influences the affinity with which a protein or peptide binds to an epitope and is herein defined as at least part of an antibody that is capable of specifically binding to an epitope. Said affinity region for instance comprises at least part of an immunoglobulin (such as in IgIV), at least part of a monoclonal antibody and/or at least part of a humanized antibody. Said affinity region preferably comprises at least part of a heavy chain and/or at least part of a light chain of an antibody. In one embodiment said affinity region comprises a double F(ab′)₂ or single form Fab fragment.

Generally, affinity regions occur on the surface of cells such as T-cells or β-cells or other immune cells, in which case they are often part of a cellular receptor. Affinity regions also occur in synthetic form in phage display libraries.

One embodiment of the invention comprises contacting a collection of immunoglobulins of an IgIV solution with a collection of misfolded proteins and/or cross-β structures, preferably with a given selected cross-β structure, and/or with a protein comprising a cross-β structure, preferably a given selected protein comprising a cross-β structure. An epitope recognized by an affinity region is in one embodiment located on a cross-β structure itself. Therefore, one embodiment of the invention provides a method according to the invention for selecting from a collection of IgIV molecules, at least one IgIV molecule comprising an affinity region that is capable of interacting with an epitope of a cross-β structure and/or with an epitope of a protein comprising a cross-β structure, wherein said epitope is at least part of a cross-β structure of a protein. In another embodiment, said epitope is exposed on said protein comprising a cross-β structure. Said epitope is not necessarily located on said cross-β structure. Generally a cross-β structure induces a different folding of a protein, which often results in the induction and/or unveiling of hitherto unknown epitopes, or the change or deletion of known epitopes of said protein. Therefore, it is also possible that a protein in which a cross-β structure is formed during misfolding, displays an epitope that is related to the presence of a cross-β structure. In one embodiment an IgIV molecule capable of specifically binding such induced and/or unveiled epitope is selected.

As described before, misfolded proteins, cross-β structures and/or (misfolded) proteins comprising a cross-β structure are an underlying cause of disease symptoms of many diseases. Said disease symptoms, related to the presence of cross-β structures, are at least partly diminished by the administration of a collection of IgIV molecules according to the present invention. A collection of IgIV molecules according to the present invention is particularly suitable for removing misfolded proteins and/or proteins or peptides comprising a cross-β structure, preferably related to and/or associated with a disease, from a sample such as for instance a body fluid or tissue sample, thereby decreasing the amount of (circulating) misfolded proteins and/or proteins or peptides comprising a cross-β structure. As used herein, the term “removing a misfolded protein and/or protein or peptide comprising a cross-β structure” comprises separating said protein and/or peptide from a sample, as well as binding, covering, shielding and/or neutralizing a misfolded protein and/or a cross-β structure and/or any other part of a protein or peptide comprising a cross-β structure, thereby at least in part preventing interaction of said misfolded protein and/or cross-β structure and/or protein or peptide comprising a cross-β structure with other binding molecules. This way, adverse effects related to the presence of a misfolded protein and/or a cross-β structure and/or to the presence of a protein or peptide comprising a cross-β structure, such as for instance infections and/or inflammation in AIDS, and/or disease symptoms of such painful and devastating diseases like for example rheumatoid arthritis and multiple sclerosis, are at least in part decreased. The same principle is also applicable to inflammatory conditions in which proteins are altered by the presence of a cross-β structure (be it a cross-β structure generated by the body or generated and/or induced by a pathogen).

Because of the low concentration and the wide variety of antibodies reacting with a misfolded protein and/or a cross-β structure and/or with a protein comprising a cross-β structure, relatively large amounts of IgIV would have to be administered to achieve a positive result, which increases the risk of adverse side effects. Furthermore, in current IgIV treatments, the body is unnecessarily stressed by the administration of a lot of proteins that have no function for the disease they are administered for. It is therefore a major advantage that a selection is now made with a method according to the invention from the pool of IgIV for IgIV molecules capable of specifically binding a misfolded protein and/or a cross-β structure and/or a protein comprising a cross-β structure. IgIV preparations according to the invention comprising enriched fractions of immunoglobulins capable of specifically binding a misfolded protein and/or a cross-β structure and/or a protein comprising a cross-β structure are particularly suitable for administering to a non-human animal or human at risk of suffering or already suffering from a cross-β structure-related disease. Because now an enriched selection of IgIV is administered, comprising IgIV molecules capable of specifically reacting with a misfolded protein and/or a cross-β structure and/or with a protein comprising a cross-β structure, it has become possible to use a total concentration of IgIV molecules which is lower than in current IgIV treatments and still have the same or even better therapeutic effect than with currently used IgIV.

An IgIV molecule comprising an affinity region that is capable of interacting with a misfolded protein and/or an epitope of a cross-β structure and/or with an epitope of a protein comprising a cross-β structure is selected from a collection of IgIV molecules in various ways. For instance, said IgIV molecule is selected by contacting a pool of IgIV molecules with a misfolded protein and/or a cross-β structure and/or with a protein comprising a cross-β structure. Subsequently, bound IgIV molecules are collected. In one embodiment said cross-β structure and/or protein comprising a cross-β structure is related to a disease. For instance, myelin, myelin basic protein and/or myelin oligodendrocyte glycoprotein is preferably used in order to select IgIV molecules for use in at least in part treating and/or preventing multiple sclerosis. Likewise, collagen and/or rheuma factor is preferably used in order to select IgIV molecules for use in at least in part treating and/or preventing rheumatoid arthritis. Various alternative methods for selecting an IgIV molecule capable of interacting with a misfolded protein and/or an epitope of a cross-β structure and/or with an epitope of a protein comprising a cross-β structure are available in the art, which are suitable for use in a method according to the invention.

In one preferred embodiment of the present invention, an IgIV molecule capable of interacting with any given misfolded protein of interest and/or with any given cross-β structure epitope of interest and/or with any given epitope of interest of a protein comprising a cross-β structure, is selected using any kind of misfolded protein, cross-β structure epitope and/or epitope of a protein comprising a cross-β structure. According to the present invention, with the use of one or several affinity matrices comprising misfolded proteins and/or proteins comprising crossbeta structure or amyloid, affinity regions are selected from any composition of affinity regions, that are capable of preferentially, selectively and with increased affinity binding to misfolded proteins and/or proteins comprising a crossbeta structure, that were not necessarily included in the set of affinity regions used for the selection. The Examples demonstrate that with the use of a solid support with immobilized selected misfolded proteins, affinity regions are isolated which have affinity for virtually any misfolded protein.

In addition, according to the present invention, both with misfolded proteins with fibrillar appearance, as well as misfolded protein aggregates lacking fibrillar features, affinity regions are selected which exhibit broad range specificity for misfolded proteins and/or proteins comprising crossbeta structure. For instance, with an Aβ fibril-affinity matrix affinity regions are selected that display affinity for non-fibrillar multimers of for example misfolded BSA-AGE, aggregates of Aβ and dOVA. At the other hand, with the use of non-fibrillar HbAGE-matrix or non-fibrillar misfolded IgIV-matrix, affinity regions are selected that efficiently bind to Aβ fibrils.

In the Examples, with the use of a bovine serum albumin-AGE-matrix, affinity regions with affinity for human Aβ, human albumin and chicken ovalbumin were selected. With the use of a human Aβ-matrix, affinity regions that are capable of binding to glycated bovine serum albumin and chicken ovalbumin were selected. With a glycated human Hb-matrix, affinity regions capable of binding to misfolded mouse IgG were selected. Hence, according to the present invention, with misfolded proteins originating from one species, affinity regions can be selected that have affinity for misfolded proteins originating from other species.

Moreover, according to the invention, from a collection of human IgIV affinity regions a selection of affinity regions originating from at least four different β-cell clones producing IgG1, IgG2, IgG3 and IgG4 iso-types, can be selected that exhibit binding properties towards a wide range of proteins, which proteins originate from various species and need not to have substantial amino-acid sequence homology, nor similar amino acid sequence length, nor overlapping or similar 3D structure in their native fold, though they share a structural feature common to misfolded proteins. The selected affinity regions with specificity for misfolded proteins and/or proteins comprising crossbeta structure are useful for a variety of applications. Below, enriched affinity regions used for therapy against protein misfolding diseases is outlined in more detail.

The methods according to the invention enable selection of, amongst other things, affinity regions that are applicable in therapeutics and/or diagnostics for diseases associated with protein misfolding. A summary outlining preferred embodiments of a method according to the invention is depicted in FIG. 26. Any misfolded protein of choice (mix X and Y in FIG. 26, representing the Misfoldome) is suitable for use to select affinity regions, but preferably misfolded proteins (mix A in FIG. 26) are used that are associated with disease. Since misfolded proteins share common characteristics, in general, affinity regions will be selected that bind to more than one particular misfolded protein. However, as disclosed in this application, it is also possible to select affinity regions that preferentially bind a subset or even a single type of misfolded protein. By combining a set of columns a person skilled in the art is able to select those affinity regions of interest that are applicable for therapeutics and/or diagnostics for misfolding in general or that are preferentially applicable for a particular disease or set of diseases in which the misfolded protein of choice is implicated. As illustrated in FIG. 26, application of column I (mix of misfolded proteins not necessarily related to a disease) will result in affinity regions (preparation 1) with affinity for misfolded proteins in general, i.e. the Misfoldome. Such affinity regions are suitable for diagnostics and also for therapy. However use of such affinity regions for therapeutic purposes implies the potential risk for side effects, due to the fact that affinity regions are introduced to the patient that not only bind to the disease-related misfolded protein (desired therapeutic effects), but also to other misfolded proteins present (unpredictable side-effects of the therapy). By combining columns I and III, and more preferably columns II and IV, those affinity regions are selected that preferentially interact with misfolded proteins specific for a disease or a set of diseases. Column IV is used to remove those affinity regions that interact with misfolded proteins which are not related to the target disease of choice. Hence, preparations 3 and 4 are preferentially selected for specific therapeutic purposes.

Hence, in order to select affinity regions capable of specifically binding misfolded proteins associated with a disease of interest, two columns are preferably used. One column (“the general column”) comprises misfolded proteins which are not necessarily associated with said disease. The other column (“the specific column”) comprises more misfolded proteins that are associated with said disease, as compared to the general column. Preferably, the misfolded proteins of said specific column essentially consist of misfolded proteins associated with said disease.

In one embodiment, the general column is firstly used. In this step, affinity regions capable of specifically binding to any misfolded protein are isolated. Subsequently, according to this embodiment, the specific column is used. In this step, the composition comprising the affinity regions is enriched in affinity regions specific for misfolded proteins associated with a disease of interest.

In another embodiment, the above mentioned columns are used in the reverse order. Firstly, the specific column is used in order to isolate affinity regions capable of specifically binding misfolded proteins associated with a disease of interest. In practice, the resulting composition will also comprise affinity regions capable of specifically binding misfolded proteins that are not associated with said disease of interest. Therefore, a general column is preferably subsequently used. An important characteristic of this second column is that it does not, or to a lower extent, comprise misfolded proteins that are associated with said disease of interest. Said second column will bind affinity regions capable of specifically binding misfolded proteins that are not associated with said disease of interest, but it will not, or to a lower extent, bind affinity regions that are specific for misfolded proteins associated with said disease of interest. Hence, the flow through fraction is enriched in affinity regions specific for misfolded proteins associated with said disease of interest.

In one embodiment selected IgIV molecules are tested for their reactivity with a given protein and/or peptide of interest in a body sample of a human or animal suffering from said disease. The capability of a selected IgIV collection according to the invention of binding a specific protein of interest from a body sample is for example measured with a blood platelet aggregation test, an opsonophagocytosis test, and/or a complement activation or inhibition test.

One further embodiment provides a selection method according to the invention wherein a misfolded protein and/or an epitope, being a cross-β structure or an epitope of a protein comprising a cross-β structure, is attached to a support such as for example spheres or particles or beads or sheets or strands of latex or agarose or Sepharose or glass or plastic or metal or any other suitable substance or compound or material or molecule to enhance the efficiency of the selection, like for instance magnetic beads. Therefore the invention provides a method as described herein wherein said misfolded protein and/or said epitope is bound to a solid support.

The invention furthermore provides a collection of IgIV molecules, enriched in IgIV molecules comprising an affinity region that is capable of specifically interacting with a misfolded protein and/or with an epitope of a cross-β structure and/or with an epitope of a protein comprising a cross-β structure. As explained above, said collection of IgIV molecules has at least one improved property as compared to currently used IgIV. A collection of IgIV molecules according to the invention is preferably selected from currently used IgIV with a selection method according to the invention. With a method of the invention, a skilled person is able to select from a large collection of IgIV, a smaller selection of IgIV molecules which is enriched in IgIV molecules comprising affinity regions capable of specifically binding a misfolded protein and/or an epitope on a cross-β structure and/or an epitope on a protein comprising a cross-β structure. Therefore, one embodiment provides a collection of IgIV molecules, enriched in IgIV molecules comprising an affinity region capable of interacting with a misfolded protein and/or an epitope of a cross-β structure and/or with an epitope of a protein comprising a cross-β structure, selected by a method according to the present invention. An enriched collection of IgIV molecules according to the invention is suitable for administering to a patient in need of such medicament in a smaller amount than currently used IgIV preparations, because of the relative increase of affinity regions in said enriched collection capable of interacting with a misfolded protein and/or with an epitope on a cross-β structure and/or with an epitope on a protein comprising a cross-β structure.

The invention further provides a composition comprising at least 5 isolated, synthetic and/or recombinant molecules comprising an affinity region that is capable of interacting with a misfolded protein and/or an epitope of a cross-β structure and/or with an epitope of a protein comprising a cross-β structure. Preferably, said composition comprises at least 8, more preferably at least 10 of the above mentioned isolated, synthetic and/or recombinant molecules. In one preferred embodiment synthetic and/or recombinant molecules are used. An advantage of a composition of synthetic and/or recombinant molecules is the fact that the need for IgIV is obviated. This is, amongst other things, advantageous because the supplies and availability of IgIV are not sufficient, and because there are certain risks involved in administration of biological products derived from human blood (such as for instance the risk of infection with prion disease and with pathogens such as hepatitis virus or HIV). Now that the present invention has provided an enriched selection of IgIV molecules according to the invention, it has become possible to generate synthetic and/or recombinant molecules with at least one similar property as said enriched selection of IgIV molecules in kind, not necessarily in amount. Once an enriched selection of certain immunoglobulins of IgIV has been made, a skilled person is able to determine by methods known in the art (such as for example, but not limited to, the Maldi-Toff method) the amino acid sequence of said immunoglobulins, or at least of an affinity region of said immunoglobulins. Said amino acid sequence is then preferably used to select or produce synthetic or partially synthetic molecules that have the same binding characteristic in kind, not necessarily in amount, as at least one affinity region of a selected IgIV molecule according to the invention. A non-limiting example of a synthetic or partially synthetic molecule is a product obtained by recombinant or chemical synthesis of peptides, proteins or other molecules. It is even possible to screen a phage display library with misfolded proteins and/or with cross-β structures and/or with proteins comprising a cross-β structure, or epitopes of said proteins, to select binding molecules having an affinity region reacting with said misfolded protein, cross-β structure and/or protein comprising a cross-β structure. Therefore, one embodiment provides a method for producing a composition according to the invention, comprising defining the amino acid sequence of an affinity region of at least one IgIV molecule capable of interacting with a misfolded protein, an epitope of a cross-β structure and/or with an epitope of a protein comprising a cross-β structure, and producing synthetic and/or recombinant molecules comprising said amino acid sequence. In another embodiment, the invention also provides a synthetic or recombinant molecule comprising an affinity region that is capable of interacting with a misfolded protein, an epitope of a cross-β structure and/or with an epitope of a protein comprising a cross-β structure, said molecule produced according to a method as described above.

Hence, affinity regions analogous as those isolated from IgIV are for instance made recombinantly or synthetically by applying standard techniques, known to a person skilled in the art, including protein sequence analysis, DNA cloning and expression technology. One embodiment of the invention comprises the following steps: (1) The amino acid sequence, at least from the variable regions of both heavy and light chains, or at least from the complementarity determining regions 1-3 (CDRs), or at least from CDR3 of the heavy chain (HC) of isolated affinity regions, is obtained by protein sequence analysis. (2) A nucleic acid sequence, preferably a DNA sequence, encoding the identified amino acids sequence is made synthetically. As an alternative to the exact sequence determined by protein analysis, a sequence can be produced wherein one or more mutations are introduced, preferably in the CDR3, and even more preferably in the CDR3 of the heavy chain (HC), in order to produce affinity regions with altered affinity, preferably increased and/or more specific affinity. (3) The nucleic acid is cloned into an appropriate expression vector. Such vector preferably already contains the sequences encoding the constant regions of immunoglobulins of the desired type, such as for instance to obtain IgG1, IgG2a, IgG2b, IgM, IgA, IgE etc. (4) Said vector is transduced in either way into an expression system of choice, preferably a mammalian cell. (5) Cells expressing the affinity region are selected. (6) Recombinantly made affinity regions are purified from said cells or cell derived culture supernatant. If mutations are introduced into the original affinity region sequence to optimize affinity, the newly made affinity regions are optionally re-selected, preferably using a method according to the present invention. Such generation of semi-synthetic affinity regions with an even increased repertoire of affinity regions, preferably in the complementarity determining regions, preferably in the CDR3, even more preferably in the CDR3 of the HC, is preferably performed by generation of a semi-synthetic library, such as a phage display library (see below).

Besides a collection of human immunoglobulins such as IVIg obtained from blood, a combinatorial library can also be obtained from any other set of affinity regions, preferably a set of recombinant affinity regions such as those present in a phage display library (Winter et al. 1994; Hoogenboom, 1992, 1997, 2000, 2002, 2005). Preferably, such a library is comprised of sequences related to mammalian affinity regions, preferably human affinity regions, like immunoglobulins. In one preferred embodiment, such a phage display library comprising a collection of affinity regions is made as follows (Winter et al. 1994, de Kruif et al. 1995a, 1995b): firstly, RNA is extracted from B cells or from a tissue comprising B cells. Subsequently, cDNA is prepared. Next, cDNA encoding the variable regions is amplified, cloned into an appropriate phagemid vector and transformed into an appropriate host, such as for example a strain of Escherichia coli. In this way affinity regions are expressed, i.e. displayed by phages, as fusion proteins on the surface of filamentous bacteriophages. A phage display library is for instance prepared from B cells obtained from a healthy mammal, preferably a human, mouse, rat or llama, or alternatively from a mammal immunized with a misfolded protein. In one embodiment, a phage display library is prepared from B cells from a mammal, preferably a human, suffering from a particular disease, preferably a misfolding disease, like for example RA. In this way, a collection of affinity regions is prepared with a specific aim to comprise those affinity regions specific for misfolded proteins. For example, in one embodiment a mouse is immunized once or several times with one or a selection of misfolded proteins (like in Example 20), B cells are isolated from the spleen and used to prepare a phage display library. In another embodiment, B cells are isolated from a human with a particular disease, for example (rheumatoid) arthritis. cDNA prepared from these B cells is then preferably used to prepare a phage display library. In such a way a phage display library is prepared to comprise affinity regions with specificity for misfolded proteins involved in the chosen misfolding disease. For example, a library is prepared with affinity regions for the Fc domain of Ig's, i.e. affinity regions like Rheumatoid Factor (RF) (van Esch et al. 2003, Clin Exp. Immunol). In the above described way a person skilled in the art is able to design and prepare a phage display library with any collection of affinity regions with emphasis on a particular disease or application.

In one embodiment a phage display library with such a collection of affinity regions with an increased repertoire is prepared synthetically (Hoogenboom, 1992, 1997, 2000, 2002, 2005; de Kruif et al. 1995a, 1995b). In this way a person skilled in the art is able to design a library comprising affinity regions of considerable additional diversity. Preferably, by implementing additional sequences in the hypervariable regions, the CDRs that interact with the antigen, additional affinity regions are made, reshaping the variable domains. Besides affinity regions obtained from human sequences, a collection of affinity regions is in one embodiment created from any other species, such as llama, camel, alpaca or camelid, to obtain affinity regions, such as llama antibodies, also referred to as nanobodies, with properties related to these species.

Thus, a phage display library and/or a collection of affinity regions is prepared in many ways, for instance from a mammal immunized with one or a set of misfolded proteins. In a particularly preferred embodiment, a phage display library and/or a collection of affinity regions is prepared from a mammal with a disease, preferably a misfolding disease. Affinity regions specific for misfolded proteins are preferably selected from a phage display library using means and methods according to the invention, preferably combined with standard procedures for isolating phages. Most straightforward, in a preferred embodiment, misfolded proteins are prepared and are immobilized, preferably according to any one of the procedures disclosed in this application, and subsequently allowed to bind phages. After extensive washing bound phages are retrieved and amplified by reinfection of host. To allow recovery of only specific phages the selection procedure is preferably repeated several times. Finally, those phages are isolated that are capable of specifically binding misfolded targets. In a particularly preferred embodiment, misfolded proteins are isolated from a tissue sample obtained from an individual or combination of individuals with a disease. For example, misfolded proteins are isolated using a protein that is capable of specifically binding to misfolded proteins comprising crossbeta structure, such as tPA, RAGE or a functional equivalent thereof (see Table 4), from synovial fluid of a patient with (rheumatoid) arthritis. In analogy, any other sample can be used.

Using approaches as described above recombinantly made affinity regions for misfolded proteins are obtained.

After selection of the appropriate phages DNA encoding the variable regions of the isolated affinity regions are preferably isolated from the phagemid DNA in order to generate full antibodies. This is easily performed according to standard procedures. The DNA is preferably cloned into vectors encoding the constant regions for the heavy and light chains. Any vector and any desired type of constant region can be used. The vector is preferably transduced in any known way into an expression system of choice, preferably a mammalian cell. Cells expressing the affinity region are preferably selected. Recombinantly made affinity regions are preferably purified from the cells or cell derived culture supernatant. In such a way any immunoglobulin affinity region for misfolded proteins is prepared (Bloemendal et al 2004; Huls et al 1999a, 1999b; Boel et al 2000).

For use in humans, “chimeric” or “humanized” recombinant affinity regions are preferably generated. Affinity regions obtained from other species are preferably modified in such a way that non-human sequences are replaced with human sequences, wherever possible, while the binding properties of the affinity region are preferably not influenced too much. In one embodiment affinity regions are made during classical immunization strategies, preferably using mice or rats, even more preferably using transgenic mice that encode human immunoglobulins. After immunization hybridoma cell lines expressing monoclonal antibodies are preferably prepared by standard procedures, and/or by applying the above described phage display technology. Monoclonal antibodies are preferably selected that are capable of specifically interacting with misfolded proteins. “Chimeric” or “humanized” versions of such affinity regions, when made using normal mice or rats, are for instance made by replacing the non-human constant regions and the relevant non-human variable regions with the relevant human homologous regions (Morrison et al 1984; Jones et al. 1986). Moreover, different constant regions are introduced when desired.

In one preferred embodiment a composition according to the invention comprises a functional part, derivative and/or analogue of at least one IgIV molecule comprising an affinity region capable of interacting with a misfolded protein and/or an epitope of a cross-β structure and/or with an epitope of a protein comprising a cross-β structure. A functional part of an IgIV molecule is defined as a compound which has the same immunological binding properties in kind, not necessarily in amount. Said functional part is capable of binding a misfolded protein and/or a cross-β structure and/or protein comprising a cross-β structure, albeit not necessarily to the same extent as said IgIV molecule. A functional derivative of an IgIV molecule is defined as an IgIV molecule which has been altered such that the capability of binding a misfolded protein and/or a cross-β structure and/or a protein comprising a cross-β structure of the resulting compound is essentially the same in kind, not necessarily in amount. A derivative is provided in many ways, for instance through conservative amino acid substitution, whereby an amino acid residue is substituted by another residue with generally similar properties (size, hydrophobicity, etc), such that the overall functioning is likely not to be seriously affected, or even improved.

A person skilled in the art is well able to generate analogous compounds of an IgIV molecule. This can for instance be done through screening of a peptide library. Such an analogue is capable of binding a misfolded protein and/or a cross-β structure and/or protein comprising a cross-β structure, albeit not necessarily to the same extent as said IgIV molecule.

A selected IgIV molecule and/or an isolated, synthetic or recombinant molecule comprising an affinity region capable of specifically binding a misfolded protein and/or an epitope of a cross-β structure and/or an epitope of a protein comprising a cross-β structure is in one embodiment of the invention used for reacting and binding to a misfolded protein and/or cross-β structures and/or proteins comprising cross-β structures in vitro. Said molecule is preferably reacted with a sample of body fluid or tissue, food, fluid, or a pharmaceutical composition comprising misfolded proteins and/or a cross-β structure and/or a protein comprising a cross-β structure, and bound material is preferably removed. Another application of a molecule according to the invention is reacting and binding to misfolded proteins and/or cross-β structures and/or proteins comprising a cross-β structure in vivo.

One preferred embodiment provides a composition according to the invention wherein at least one of said molecules further comprises a misfolded protein and/or a cross-β structure binding compound. A misfolded protein and/or cross-β structure binding compound is a compound capable of specifically binding a misfolded protein and/or a cross-β structure. A misfolded protein and/or cross-β structure binding molecule is capable of serving as an effector molecule by enhancing the capability of a molecule of a composition according to the invention to specifically bind a misfolded protein and/or a cross-β structure or a protein comprising a cross-β structure. Enhanced binding of a misfolded protein and/or a cross-β structure due to said cross-β structure-binding molecule is for instance desired for enhancing the formation and removal of misfolded protein and/or cross-β structure complexes from the circulation and/or from the body. Alternatively, or additionally, local accumulation of a misfolded protein and/or cross-β structures such as present in amyloid plaques is diminished.

Non-limiting examples of misfolded protein and/or cross-β structure binding molecules are a finger domain (also referred to as fibronectin type I domain) of tissue-type plasminogen activator (tPA), hepatocyte growth factor activator (HGFA), factor XII, or fibronectin, or members of the multiligand receptor family such as receptor for advanced glycation end-products (RAGE), or low density lipoprotein receptor related protein (LRP) or CD36. Such a misfolded protein and/or cross-β structures binding molecule may even be a non-proteinaceous molecule, such as for example a dye (Congo red or Thioflavin).

In one embodiment, an effector molecule is provided to an isolated, synthetic and/or recombinant molecule of the invention and/or to a selected IgIV immunoglobulin of the invention. A composition and a collection of IgIV molecules according to the invention, wherein at least one of said molecules further comprises an effector molecule, is therefore also provided. In one preferred embodiment said effector molecule comprises an inhibitor of misfolding, such as for instance Congo red. In another preferred embodiment said effector compound is capable of enhancing the complement system and/or the phagocytic system of an animal preferably a human) in order to enhance removal of (proteins comprising) undesired cross-β structures. Hence, in one preferred embodiment said effector compound comprises a complement activating factor such as for instance, but not limited to, any complement protein, a complement activating cytokine, C reactive protein, serum amyloid P component, Pentraxin-3, an Fc region of immunoglobulins (ligand for C1q), a complement control protein, a molecule capable of enhancing the complement activating activity of complement control proteins, and/or a molecules capable of inhibiting the inhibitory activity of complement control proteins. Non-limiting examples of complement control proteins are C1-inhibitor, C4 binding protein, factor H, factor I, properdin, S protein, complement receptor type I, membrane cofactor protein, decay accelerating factor, C8 binding protein and CD59. In a further preferred embodiment said effector compound is capable of facilitating breakdown of a misfolded protein and/or a cross-β structure and/or a protein comprising a cross-β structure. Another preferred property of said effector compound is a capability of facilitating cellular uptake of a misfolded protein and/or a cross-β structure and/or a protein comprising a cross-β structure. One embodiment provides a composition according to the invention, wherein said isolated, synthetic and/or recombinant molecule, or said selected IgIV molecule, comprises an effector compound which is a protease or a misfolded protein and/or cross-β structure-binding part thereof. Said effector is particularly suitable for binding and/or breaking down a misfolded protein and/or a cross-β structure and/or an undesired protein comprising a cross-β structure. In a further preferred embodiment said effector compound comprises an immunopotentiating compound in order to enhance an immune response directed against a misfolded protein and/or a cross-β structure and/or a protein comprising a cross-β structure. Said immunopotentiating compound preferably comprises a cytokine.

In a further embodiment said effector compound comprises a misfolded protein and/or cross-β structure binding-potentiating factor. This is a factor capable of enhancing the capability of a molecule according to the invention of binding a misfolded protein and/or cross-β structure and/or binding a protein comprising a cross-β structure. Non-limiting examples of such factors are Thioflavin T and Thioflavin S (See for instance example 4).

In a further embodiment said effector compound comprises a clearance signal that aids in removal of the resulting complex after a molecule and/or IgIV molecule of the invention has bound a misfolded protein and/or a cross-β structure and/or a protein comprising a cross-β structure. Clearance signals are well known in the art. A preferred example of a clearance signal is at least part of an Fc region, more preferably an Fcγ region capable of interacting with an Fc receptor (preferably with an FcγIIb receptor). Said clearance signal is capable of enhancing removal of a complex comprising a molecule according to the invention bound to a misfolded protein and/or a cross-β structure or to a protein comprising a cross-β structure from the circulation and/or from the body of an animal preferably a human).

Activation of the complement system results in a cascade of reactions, including inflammation, cell destruction and tissue damage. In some circumstances it is desired to counteract the complement system in order to dampen adverse side effects. Non-limiting examples of such circumstances are situations with excessive and/or uncontrolled activation of the complement system or (sustained) activation of the complement system without a properly functioning negative feedback mechanism or overstimulation of the complement system, for instance due to sustained and/or overexpressed levels of activators, like for example during inflammation, amyloidoses and/or rheumatoid arthritis. In one embodiment an effector compound is therefore used that is an inflammation suppressive compound, preferably a complement inhibiting factor such as for instance an immunoglobulin or a compound capable of at least partly inhibiting or blocking important functioning of complement proteins and/or capable of at least partly inhibiting or blocking important functioning of any protein or compound that comprises complement system stimulatory capacities. Non-limiting examples of complement inhibiting factors are soluble TNF receptor, IL-1 receptor antagonists and anti-inflammatory cytokines.

In yet another embodiment said effector compound comprises an opsonizing compound. Additionally, or alternatively, said isolated, synthetic and/or recombinant molecule of the invention is itself an opsonizing compound. Opsonizing is defined herein as a process of inducing and/or enhancing phagocytosis of a substance by phagocytes such as macrophages, polymorphonuclear cells and the like. Some substances are capable of withstanding and/or escaping phagocytosis, for instance due to the nature of their surface. In such cases, phagocytosis is preferably induced and/or enhanced by opsonizing binding compounds, that, once attached to a substance, facilitate the uptake of said substance by phagocytes such as macrophages and polymorphonuclear cells and the like.

In one embodiment it is determined whether a selected IgIV molecule and/or an isolated, synthetic and/or recombinant molecule according to the invention has an opsonizing capacity, using phagocytic cells. According to this embodiment, once an enriched selection of IgIV molecules according to the invention has been provided, said collection is preferably incubated with a misfolded protein and/or a cross-β structure and/or a protein comprising a cross-β structure, where after complexes of IgIV molecules bound to a misfolded protein and/or a cross-β structure and/or a protein comprising a cross-β structure are subsequently contacted with a phagocytic cell in order to determine which IgIV molecules are capable of inducing and/or enhancing phagocytosis of said misfolded protein and/or cross-β structure and/or protein comprising a cross-β structure. It is of course also possible to perform the same kind of test with isolated, synthetic and/or recombinant molecules according to the invention. Further provided is therefore a method for selecting from a collection of IgIV molecules according to the invention, or from a composition according to the invention, a molecule comprising an affinity region which is capable, upon interacting with a misfolded protein and/or an epitope of a cross-β structure and/or upon interacting with an epitope of a protein comprising a cross-β structure, of inducing opsonization of said misfolded protein and/or cross-β structure and/or a protein comprising a cross-β structure by a phagocytic cell, said method comprising:
contacting a collection of IgIV molecules according to the invention, and/or a composition according to the invention, with a misfolded protein and/or a cross-β structure and/or with a protein comprising a cross-β structure;
contacting any complex comprising a misfolded protein and/or a cross-β structure and/or a protein comprising a cross-β structure, bound to an IgIV molecule and/or to an isolated, synthetic and/or recombinant molecule, with a phagocytic cell; and
collecting an IgIV molecule and/or isolated, synthetic and/or recombinant molecule that is capable of inducing or enhancing phagocytosis, by a phagocytic cell, of said misfolded protein and/or cross-β structure and/or a protein comprising a cross-β structure.

Said test is preferably performed in vitro. Selected IgIV molecules and/or isolated, synthetic and/or recombinant molecules capable of inducing or enhancing phagocytosis are preferably used in order to induce and/or enhance opsonization of misfolded proteins and/or cross-β structures and/or proteins comprising a cross-β structure that are capable of withstanding and/or escaping phagocytosis. Such misfolded proteins and/or cross-β structures and/or proteins comprising a cross-β structure capable of withstanding and/or escaping phagocytosis for instance occur in disease states in which molecules capable of inducing or enhancing phagocytosis are absent or present at reduced (functional) levels, like for example in AIDS, SCIDS and a-gammaglobulinaemia, and for instance in disease states in which formation of misfolded proteins and/or cross-β structures and/or proteins comprising a cross-β structure is increased like for example in TSE, amyloidoses, diabetes, thrombosis and inflammation.

As described before, misfolded proteins and/or cross-β structures in proteins are often related to, and/or associated with, a risk and/or presence of disease, such as for instance Huntington's disease, amyloidosis type disease, atherosclerosis, diabetes, bleeding, thrombosis, cancer, sepsis and other inflammatory diseases, rheumatoid arthritis, transmissible spongiform encephalopathies such as Creutzfeldt-Jakob disease, Multiple Sclerosis, auto-immune diseases, diseases associated with loss of memory such as Alzheimer's disease, Parkinson's disease and other neuronal diseases (epilepsy), encephalopathy and systemic amyloidoses. An enriched collection of IgIV molecules according to the invention and a collection of isolated, synthetic and/or recombinant molecules according to the invention, being capable of specifically binding a misfolded protein and/or cross-β structures and/or proteins comprising a cross-β structure, are particularly suitable for at least in part preventing and/or treating such misfolded protein and/or cross-β structure related and/or associated diseases. One embodiment therefore provides a collection of IgIV molecules according to the invention and/or a composition according to the invention for use as a medicament and/or prophylactic agent. The invention furthermore provides a use of a collection of IgIV molecules and/or a composition according to the invention for the preparation of a medicament and/or prophylactic agent. Said medicament and/or prophylactic agent is particularly suitable for at least in part preventing, treating and/or stabilizing diseases that are related to and/or associated with occurrence of misfolded proteins and/or cross-β structures, blood coagulation disorders, sepsis, inflammation, and/or an infection by a microbe, pathogen, bacterium, parasite and/or virus. Further provided is therefore a use of a collection of IgIV molecules according to the invention and/or a composition according to the invention for the manufacture of a medicament for at least partial prevention and/or treatment of a misfolded protein and/or cross-β structure related and/or associated disease, a blood coagulation disorder, sepsis, inflammation and/or a microbial/pathogen/parasite/bacterial/viral infection. A method for at least partial prevention and/or treatment of a misfolded protein and/or cross-β structure related and/or associated disease, a blood coagulation disorder, sepsis and/or a microbial/pathogen/parasite/bacterial/viral infection in an individual, comprising administering a collection of IgIV molecules according to the invention and/or a composition according to the invention to said individual, is also herewith provided.

In one preferred embodiment said microbial/pathogen/parasite/bacterial/viral infection comprises an opportunistic infection. This is an infection by an organism such as for instance a pathogen and/or virus that does not ordinarily cause disease but that, under certain circumstances (such as an impaired immune system), becomes pathogenic. An impaired immune system is for instance caused by medication such as chemotherapy. In a particularly preferred embodiment said microbial/pathogen/parasite/bacterial/viral infection comprises an HIV-related opportunistic infection. Since opportunistic infections are the major cause of death in HIV patients, it is highly desired to provide medicaments and/or prophylactic agents against such infections. Many opportunistic infections involve the presence of a misfolded protein and/or a cross-β structure. For instance, amyloid structures occur on the surface of microbial organisms like fungi, yeast and bacteria. Said amyloid-like structures are generally called hydrophobins on fungi, chaplins on gram-positive bacteria, and curli or tafi or aggregative fimbriae on gram-negative bacteria. Since an enriched collection of IgIV molecules according to the invention and a collection of isolated, synthetic and/or recombinant molecules according to the invention are particularly suitable for binding such misfolded proteins and/or cross-β structures and/or proteins comprising a cross-β structure, said collections of the invention are particularly suitable for counteracting and/or at least in part preventing HIV-related opportunistic infections. The invention therefore provides a method for at least partial prevention and or treatment of an HIV-related opportunistic infection in an individual, comprising administering a collection of IgIV molecules according to the invention and/or a composition according to the invention to said individual.

A composition comprising a collection of IgIV molecules according to the invention and/or a composition according to the invention and a suitable carrier, diluent and/or excipient is also herewith provided. Said composition preferably comprises a pharmaceutical composition. In order to be able to administer a medicament according to the present invention to a patient in need of treatment, said medicament must fulfil the needs for a pharmaceutically acceptable formulation. This means that a medicament according to the invention comprises an enriched collection of IgIV molecules according to the invention and/or a collection of isolated, synthetic and/or recombinant molecules according to the invention which are of pharmaceutical grade, physiologically acceptable and tested for extraneous agents. A pharmaceutical composition comprising an enriched collection of IgIV molecules according to the invention and/or a collection of isolated, synthetic and/or recombinant molecules according to the invention and a pharmaceutically acceptable carrier, diluent and/or excipient is also herewith provided. Preferably, said composition comprises a misfolded protein and/or cross-β structure-binding compound in order to enhance interaction of said pharmaceutical composition with a misfolded protein and/or a cross-β structure and/or with a protein comprising a cross-β structure. Therefore, the invention provides a composition according to the invention further comprising a misfolded protein and/or cross-β structure-binding compound. In a further preferred embodiment of the invention, binding of a composition according to the invention to a misfolded protein and/or a cross-β structure and/or to a protein comprising a cross-β structure is further enhanced or potentiated by the addition of a compound that is known for its misfolded protein and/or cross-β structure-binding-potentiating characteristics, such as for example dye molecules such as Thioflavin T or Thioflavin S. Therefore, the present invention discloses a composition according to the invention further comprising a misfolded protein and/or cross-β structure-binding-potentiating compound.

In another preferred embodiment, removal of misfolded proteins and/or cross-β structures and/or proteins comprising a cross-β structure from a body is enhanced by adding to a composition according to the invention complement potentiating signals capable of enhancing complement activation. Therefore, the invention provides a composition according the invention, further comprising a complement potentiating compound.

Since activation of the complement system results in a cascade of reactions, including inflammation, cell destruction and tissue damage, it is sometimes desired to at least in part counteract complement activation. In some cases, activation of the complement system in relation to the clearance of misfolded proteins and/or cross-β structures is itself causing illness. In such cases, a composition according to the invention preferably further comprises a complement inhibiting compound. In one embodiment a composition according to the invention comprises an inflammation suppressive compound.

The present invention furthermore provides means and methods for increasing extracellular protein degradation and/or protein clearance in an individual. In a natural situation, the formation of a misfolded protein and/or cross-β structures initiates and/or participates in a physiological cascade of events, dealing with removal of unwanted molecules, such as for instance misfolded proteins, apoptotic cells or even pathogens. This pathway regulates the removal of unwanted biomolecules during several processes, including protein misfolding during synthesis in the endoplasmic reticulum, fibrinolysis, formation of neuronal synaptic networks, clearance of used, unwanted and/or destroyed (denatured) proteins, induction of apoptosis and clearance of apoptotic cells, necrotic cells, aged cells and/or pathogens. Since a collection of IgIV molecules according to the invention and a composition according to the invention are particularly suitable for binding misfolded proteins and/or cross-β structures and proteins comprising cross-β structures, extracellular protein degradation and/or protein clearance is increased. Further provided is therefore a method for increasing extracellular protein degradation and/or protein clearance in an individual, comprising administering a collection of IgIV molecules according to the invention and/or a composition according to the invention to said individual.

By binding and removing misfolded proteins and/or cross-β structures and proteins comprising cross-β structures, a collection of IgIV molecules according to the invention and a composition according to the invention are capable of at least in part counteracting misfolded protein and/or cross-β structure mediated effects in an individual. Further provided is therefore a method for at least in part inhibiting misfolded protein and/or cross-β structure mediated effects in an individual, comprising administering an effective amount of a collection of IgIV molecules according to the invention and/or a composition according to the invention to an individual.

In a preferred embodiment, a collection of IgIV molecules according to the invention and/or a composition according to the invention is used in order to inhibit platelet aggregation that is induced by misfolded proteins and/or proteins comprising a cross-β structure. An example of such use is shown in Example 2. Therefore, the invention provides a use of a collection of IgIV molecules according to the invention and/or a composition according to the invention for inhibiting protein-induced blood-platelet aggregation.

In another preferred embodiment, a collection of IgIV molecules according to the invention and/or a composition according to the invention is used in order to compete binding of the serine protease tissue type plasminogen activator (tPA) to a misfolded protein and/or a cross-β structure and/or to a protein comprising a cross-β structure. tPA induces the formation of plasmin through cleavage of plasminogen. Plasmin cleaves fibrin and this occurs during lysis of a blood clot. Although not essential for fibrinolysis in mice, tPA has been recognized for its role in fibrinolysis for a long time. Activation of plasminogen by tPA is stimulated by fibrin or fibrin fragments, but not by its precursor, fibrinogen. tPA is a misfolded protein and cross-β structure binding protein, a multiligand receptor and a member of the cross-β structure pathway. tPA mediates a misfolded protein and/or cross-β structure induced cell dysfunction and/or cell toxicity. tPA mediates at least in part cell dysfunction and/or toxicity through activation of plasminogen. The plasminogen dependent effects are inhibited with a collection of IgIV molecules according to the invention and/or a composition according to the invention. Excessive or uncontrolled tPA/plasminogen activation during a disease state is treated this way. Non-limiting examples of such disease states are Alzheimer's disease, infections, preeclampsia, angina pectoris, inflammatory and noninflammatory joint diseases, diabetes.

One preferred embodiment provides a use of a collection of IgIV molecules and/or a composition according to the invention for at least partial removal of a misfolded protein and/or cross-β structures and/or proteins comprising a cross-β structure from a sample. Removal of a misfolded protein and/or cross-β structures and/or proteins comprising a cross-β structure is desired in a variety of applications. For instance, if an individual is suffering from, or at risk of suffering from, a disorder related to and/or associated with the presence of a misfolded protein and/or a cross-β structure, removal of such misfolded protein and/or cross-β structure from the body is beneficial in order to counteract such disorder and/or to alleviate adverse side effects. Moreover, it is advantageous to remove misfolded proteins and/or cross-β structures and/or proteins comprising a cross-β structure from products intended for (human) consumption in order to at least in part avoid uptake of misfolded proteins and/or cross-β structures. One embodiment therefore provides a method for at least partially removing misfolded proteins and/or cross-β structures and/or proteins comprising a cross-β structure from a sample, said method comprising contacting a sample with a collection of IgIV molecules according to the invention and/or a composition according to the invention, and removing from said sample any complexes of a misfolded protein and/or cross-β structures, and/or proteins comprising a cross-β structure, bound to an IgIV molecule and/or an isolated, synthetic and/or recombinant molecule. Said sample preferably comprises a fluid sample. In one embodiment said fluid comprises a food substance.

In one preferred embodiment said sample comprises a body fluid. This embodiment is particularly suitable for at least in part preventing and/or treating a misfolded protein and/or cross-β structure related and/or associated disorder of an animal, preferably of a human individual. In one preferred embodiment extracorporeal dialysis is applied. For example, a patient suffering from a misfolded protein and/or cross-β structure related and/or associated disorder is subjected to dialysis of his blood. A collection of IgIV molecules and/or a composition according to the invention is for instance coupled to a carrier or support and/or to the inside of a tube used for dialysis. This way, misfolded proteins and/or cross-β structures and proteins comprising a cross-β structure will be removed from the blood stream of said patient, thereby at least in part relieving said patient of negative effects related to, and/or associated with, said misfolded proteins and/or cross-β structures and/or proteins comprising a cross-β structure. As another example, such use is applied in haemodialysis of kidney patients. A separation device for carrying out a method according to the invention is also provided. One embodiment thus provides a separation device for carrying out a method according to the invention, said device comprising a system for transporting (circulating) fluids, said system being provided with means for connecting to a flowing fluid, preferably to an individual's circulation, means for entry of fluid into said system and return of fluid from said system, preferably to an individual's circulation, said system further comprising a solid phase, said solid phase comprising a collection of IgIV molecules according to the invention and/or a composition according to the invention. Said separation device preferably comprises a dialysis apparatus.

Another preferred embodiment provides a use of a collection of IgIV molecules and a composition according to the invention for at least partial removal of misfolded proteins and/or cross-β structures and/or proteins comprising a cross-β structure from a pharmaceutical or any of its constituents. Important categories of nowadays pharmaceutical compositions comprising a protein or a proteinaceous compound as an active substance include, but are not limited to hormones, enzymes, vaccines and antigens, cytokines and antibodies. In addition to the above-mentioned proteinaceous pharmaceutical compositions, a large number of pharmaceutical compositions are manufactured with the help of a production and/or purification step comprising proteins. For example, many pharmaceutical compositions comprise one or more proteins as a stabilizing agent. Health problems related to the use of pharmaceutical compositions are for example related to the fields of haematology, fibrinolysis and immunology. An incomplete list of observed side-effects after administration of pharmaceutical compositions comprises for example fever, anaphylactic responses, (auto)immune responses, disturbance of haemostasis, inflammation, fibrinolytic problems, including sepsis and disseminated intravascular coagulation (DIC), which can be fatal. Said side effects are for instance caused by either an alteration of a protein or a proteinaceous compound present in said pharmaceutical composition, or by added diluents or carrier substances of said pharmaceutical composition. Alteration of a proteinaceous compound of a pharmaceutical composition comprises for example denaturation, multimerization, proteolysis, acetylation, glycation, oxidation, unfolding or misfolding of proteins. Unfolding or misfolding of initially properly folded native proteins leads to the formation of toxic structures in said proteins. Toxic structures of pharmaceutical compositions often comprise misfolded proteins and/or cross-β structures. Said toxic structures are at least in part removed with a collection of IgIV molecules and/or a composition according to the invention.

Provided is therefore a method for removing a misfolded protein and/or a cross-β structure and/or protein comprising a cross-β structure from a pharmaceutical composition or any of its constituents comprising a protein, said method comprising:
contacting said pharmaceutical composition or any of its constituents comprising a protein with a collection of IgIV molecules according to the invention and/or with a composition according to the invention;
allowing binding of said misfolded protein and/or cross-β structure and/or protein comprising a cross-β structure to said collection of IgIV molecules and/or composition; and
separating bound misfolded protein and/or cross-β structure and/or bound protein comprising a cross-β structure from said pharmaceutical composition or any of its constituents comprising a protein.

By removing a misfolded protein and/or a cross-β structure and/or a protein comprising a cross-β structure from a pharmaceutical composition, undesired side effects are at least in part decreased and/or prevented. Also provided is therefore a method for decreasing and/or preventing undesired side effects of a pharmaceutical composition and/or increasing the specific activity per gram protein, said method comprising removing an unfolded protein, an unfolded peptide, a misfolded protein, a denatured protein, an aggregated protein, an aggregated peptide, a multimerized protein and/or a multimerized peptide, and/or a peptide comprising a cross-β structure, from said pharmaceutical composition or any of its constituents, using a method according to the invention.

A pharmaceutical composition or any of its constituents comprising a protein, obtainable by a method according to the invention is also herewith provided. Said pharmaceutical composition involves a reduced risk of undesired side effects as compared to untreated pharmaceutical compositions.

In one embodiment a misfolded protein and/or a cross-β structure and/or protein comprising a cross-β structure is removed from a sample using a collection of IgIV molecules and/or a composition of isolated, synthetic and/or recombinant molecules according to the invention, wherein said collection and/or composition is bound to a solid support. This provides the advantage that a continuous process has become possible, wherein said solid support is incubated with a sample. Subsequently, said sample and said solid support are easily separated from each other, said solid support comprising misfolded proteins and/or cross-β structures and/or proteins comprising a cross-β structure that are (indirectly) bound, while the resulting sample has a lowered concentration of misfolded proteins and/or cross-β structures and/or proteins comprising a cross-β structure.

In yet another embodiment, a selected IgIV immunoglobulin and/or an isolated, synthetic and/or recombinant molecule according to the invention is used to make a diagnostic kit. Said diagnostic kit is particularly suitable for diagnosis of a disease that is related to, and/or associated with, the presence of misfolded proteins and/or cross-β structures. Said kit preferably comprises at least one affinity region of a collection of IgIV molecules according to the invention, and/or at least one affinity region of a composition according to the invention, capable of interacting with a misfolded protein and/or a cross-β structure and/or with a protein comprising a cross-β structure, and a way of visualization of an interaction of said misfolded protein and/or cross-β structure and/or said protein with said affinity region.

With such diagnostic kit, not only diseases that are generally related to and/or associated with the presence of misfolded proteins and/or cross-β structures are diagnosed, but also a more defined diagnosis is possible, dependent of the specificity of the affinity regions in the kit. A diagnostic kit capable of specifically diagnosing one kind of disorder is for instance generated by providing said kit with affinity regions which are capable of specifically binding a given misfolded protein and/or cross-β structure and/or a given protein comprising a cross-β structure that is specific for said one kind of disorder, such as for example proteins related to rheumatoid arthritis, SLE or other autoimmune diseases, or inflammatory reactions. Therefore, in one embodiment, the invention provides a diagnostic kit as described above, wherein said misfolded protein and/or cross-β structure is a disease-related misfolded protein and/or cross-β structure.

Since misfolded proteins and/or cross-β structures and proteins comprising a cross-β structure are effectively bound to a collection of IgIV molecules according to the invention and/or to a composition according to the invention, they are effectively separated and/or isolated from a sample and/or an animal's or human's body and subsequently identified. In yet another embodiment therefore, a selected IgIV immunoglobulin and/or an isolated, synthetic and/or recombinant molecule according to the invention is used to isolate misfolded proteins and/or cross-β structures and/or proteins comprising a cross-β structure. Preferably, misfolded proteins and/or cross-β structures and/or proteins comprising a cross-β structure present in a body fluid, like for example blood, serum, plasma, cerebrospinal fluid, synovial fluid, sputum and/or urine, is identified. For instance, the presence and/or identity of a misfolded protein and/or a cross-β structure, and/or protein comprising a cross-β structure, of healthy individuals is compared with the presence and/or identity of a misfolded protein and/or a cross-β structure, and/or protein comprising a cross-β structure, from individuals with a disease related to and/or associated with a misfolded protein and/or a cross-β structure and/or a protein comprising a cross-β structure. The identity and the relative concentration of a misfolded protein and/or a cross-β structure and/or protein comprising a cross-β structure is determined using any method known to a person skilled in the art, like for example, but not limited to, 2D gel electrophoresis and/or mass-spectrometric analyses. The results of a sample originating from a healthy individual and a sample originating from a patient are preferably compared. In this way, information is obtained, for instance about the identity and/or susceptibility of proteins prone to misfold and/or adopt cross-β structure conformation during defined disease states. This obtained information subsequently serves as a diagnostic tool, for instance to monitor disease state, to monitor effectiveness of therapy, to monitor occurrence of disease, and provides valuable leads for development of therapeutics targeted at misfolded proteins and/or cross-β structures and/or protein(s) comprising a cross-β structure which are preferably specific for a defined disease.

The invention therefore provides a method for determination of the identity of a misfolded protein and/or a cross-β structure or a protein comprising a cross-β structure in a sample comprising a protein, said method comprising:
contacting said sample with a collection of IgIV molecules according to the invention, and/or a composition according to the invention, resulting in bound misfolded proteins and/or cross-β structures and/or bound protein(s) comprising a cross-β structure, and
identifying a bound misfolded protein and/or cross-β structure and/or a bound protein comprising a cross-β structure. Said bound misfolded protein and/or cross-β structure and/or bound protein comprising a cross-β structure is preferably identified by analyzing at least part of the amino acid sequence of said misfolded protein and/or cross-β structure and/or protein using any method known in the art. Said sample preferably comprises an aqueous solution, more preferably a body fluid. In one preferred embodiment body fluids originating from healthy individuals (preferably humans) and body fluids originating from individuals suffering from, or suspected to suffer from, a disease related to and/or associated with the presence of a misfolded protein and/or a cross-β structure are used in order to compare a healthy state with a diseased state (or a state wherein the risk of disease is enhanced).

Because the present invention provides a way of selecting from a collection of IgIV those immunoglobulins that have affinity regions capable of interacting with a misfolded protein and/or a cross-β structure and/or with a protein comprising a cross-β structure, a skilled person is now also capable of using said selected IgIV, and/or isolated, synthetic and/or recombinant molecules according to the invention, in order to determine whether a protein or peptide which is misfolded and/or which comprises a cross-β structure is present in a sample. Provided is therefore a method for determining whether a misfolded protein and/or a protein and/or peptide comprising a cross-β structure is present in an aqueous solution comprising a protein, said method comprising:
contacting said aqueous solution with a collection of IgIV molecules according to the invention, and/or a composition according to the invention, and
detecting whether bound misfolded protein and/or bound protein and/or peptide comprising a cross-β structure is present. Said protein and/or peptide is preferably detected in an aqueous solution by contacting said aqueous solution with a collection and/or composition of the invention and detecting bound peptides and/or proteins. Provided is thus a method for detecting a misfolded protein and/or a protein and/or peptide comprising a cross-β structure in an aqueous solution comprising a protein, said method comprising contacting said aqueous solution with a collection of IgIV molecules according to the invention, and/or a composition according to the invention, resulting in bound misfolded protein and/or a bound protein and/or peptide comprising a cross-β structure, and detecting bound misfolded protein and/or protein and/or peptide comprising a cross-β structure. Binding of said collection and/or composition of the invention to a misfolded protein and/or a cross-β structure is preferably detected by means of a visualization reaction as for example by fluorescent staining or an enzymatic or colorimetric detection, or by any other visualization system available to a skilled person.

Said aqueous solution preferably comprises a detergent, a food product, a food supplement, a cell culture medium, a commercially available protein solution used for research purposes, blood, a blood product, a body fluid like for example urine, cerebrospinal fluid, synovial fluid, lymph fluid and/or sputum, a cosmetic product, a cell, a pharmaceutical composition or any of its constituents comprising a protein, or a combination of any of these.

A use of a collection of IgIV molecules according to the invention, and/or a composition according to the invention, for determining the presence of accumulated deposited misfolded protein and/or proteins with a cross-β structure, is also herewith provided. Preferably, the presence of a misfolded protein involved in a conformational disease is detected. A conformational disease is defined as a disease that is caused by, related to and/or associated with misfolding of proteins and/or conformational change of proteins.

One embodiment furthermore comprises detection of the amount of a misfolded protein and/or a cross-β structure and/or a protein comprising a cross-β structure in a composition. This is for instance done in order to determine the course of a disease. Further provided is therefore a method for determining the amount of a misfolded protein and/or a cross-β structure and/or protein comprising a cross-β structure in a composition, preferably in a medicament and/or vaccine, comprising contacting said composition with a collection of IgIV molecules according to the invention, and/or with a composition according to the invention, and relating the amount of bound misfolded protein and/or cross-β structures and/or proteins comprising a cross-β structure to the amount of cross-β structures and/or proteins comprising a cross-β structure present in said composition.

Since misfolded proteins and/or proteins comprising a cross-β structure are effectively bound to a collection of IgIV molecules according to the invention and to a composition according to the invention, they are effectively removed from a sample and/or an animal's body (preferably a human's body). This way, accumulation of misfolded proteins is diminished. Further provided is therefore a use of a collection of IgIV molecules according to the invention, and/or a composition according to the invention, for diminishing accumulation of misfolded protein and/or proteins comprising a cross-β structure. Said misfolded protein and/or proteins comprising a cross-β structure are preferably involved in a conformational disease. Diminishing accumulation of such proteins results in alleviation of symptoms of said conformational disease and/or at least partial treatment and/or prevention of the course of disease. Said conformational disease preferably comprises an amyloidosis type disease, atherosclerosis, diabetes, bleeding, thrombosis, cancer, sepsis and other inflammatory diseases, rheumatoid arthritis, transmissible spongiform encephalopathies, Multiple Sclerosis, auto-immune diseases, disease associated with loss of memory or Parkinson's disease and other neuronal diseases (epilepsy), encephalopathy, and/or rheuma.

Coagulation of blood and blood platelet clot formation also involves the presence of a misfolded protein and/or cross-β structures. Examples of the role of misfolded proteins and/or (misfolded) proteins comprising cross-β structures are activation of platelets and induction of platelet aggregation and agglutination, activation of endothelium resulting in tissue factor expression and exposure to blood, resulting in blood coagulation, and activation of the contact system of blood coagulation via activation of factor XII. In addition, during blood coagulation fibrin polymers with cross-β structure conformation are formed. The cross-β structure building block of a fibrin network subsequently serves as the binding site for tPA to localize tPA at the site where fibrinolytic activity is required. Since a collection and composition according to the invention are capable of specifically binding and/or removing misfolded proteins and/or cross-β structures and/or proteins comprising cross-β structures, said collection and composition are particularly suitable for interfering in coagulation of blood and/or clot formation and/or activation of tissue factor. Further provided is therefore a method for interfering in coagulation of blood and/or clot formation comprising providing to blood a collection of IgIV molecules according to the invention, and/or a composition according to the invention.

Also provided is a method for determining a difference in the cross-β structure content of a protein in a reference sample compared to the cross-β structure content of said protein in a test sample, wherein said test sample has been subjected to a treatment that is expected to have an effect on the cross-β structure content of said protein, the method comprising:
determining in a reference sample the cross-β structure content of a protein using a collection of IgIV molecules according to the invention and/or a composition according to the invention;
subjecting said protein to a treatment that is expected to have an effect on the cross-β structure content of said protein, thus obtaining a test sample;
determining in the obtained test sample the cross-β structure content of said protein using a collection of IgIV molecules according to the invention, and/or a composition according to the invention; and
determining whether the cross-β3 structure content of said protein in said reference sample is significantly different from the cross-β structure content of said protein in said test sample.

This embodiment is particularly suitable for determining whether a certain circumstance and/or treatment has an effect on the cross-β structure content of a protein. Once this has been determined, it is possible to select a circumstance and/or treatment that has a low capability of inducing and/or enhancing cross-β structure conformation. Of course, it is also possible to choose a circumstance and/or treatment that is well capable of inducing and/or enhancing cross-β structure conformation, depending on a particular application.

The invention is further explained by the following examples, without being restricted to them.

### EXAMPLES

### Materials & Methods

### Materials

Human broad spectrum immunoglobulin G (IgG) antibodies, referred to as ‘intravenous Ig’ (‘IVIg’ or ‘IgIV’), ‘gammaglobulin’, ‘intravenous immune globulin’, ‘intravenous immunoglobulin’ or otherwise, were obtained from the local University Medical Center Utrecht pharmacy department. Octagam from Octapharma (Octapharma International Services N.V., Brussel, Belgium; dosage 2.5 gr. in 50 ml, lot 4270568431, exp. May 2006, hereinafter referred to as IgIV ‘manufacturer I’, or IgIV (I) or IgIV-I) and Hyland Immuno Gammagard S/D IVIg from Baxter (Baxter B. V., Utrecht, The Netherlands; dosage 5 gr. with 96 ml reconstitution solution, lot LE08E044AL, exp. April 2007, hereinafter referred to as IgIV ‘manufacturer II’, IgIV (II) or IgIV-II) were used. Gammagard was reconstituted under sterile conditions by adding the supplied 96 ml H₂O and leaving the solution for 30′ on a roller device at room temperature (final IgG concentration 52 mg/ml. A clear solution was obtained without foam formation. The reconstituted solution was aliquoted and stored at −20° C. After reconstitution, the Gammagard solution contains 0.06 gr. pasteurized human albumin, 0.45 gr. glycine, 0.175 gr. NaCl, 0.43 gr. glucose-monohydrate and 0.04 gr. polyethylene glycol 3,350. Octagam is supplied as a ready-to-use solution comprising 50 mg/ml IgIV. Other components are 100 mg/ml maltose and less than 5 μg/ml Triton X-100 and less than 1 μg/ml tri-n-butyl phosphate. It is stored at 4° C. According to the manufacturer, Octagam mainly consists of IgG's (≧95%), with a minor IgA fraction (≦0.4%). The distribution over the four IgG isotypes is: IgG1, 62.6%; IgG2, 30.1%; IgG3, 6.1%; IgG4, 1.2%. Gammagard and Octagam are used at room temperature. Solutions were kept at room temperature for at least 30′ before use. Frozen aliquots of Gammagard were first quickly thawed to approximately 0° C. and then left at room temperature. A third source of human immunoglobulins was normal pooled citrated plasma of approximately 40 apparently healthy donors, prepared at the University Medical Center Utrecht. This plasma was mixed directly after the blood was drawn, and directly aliquoted and frozen at −80° C. Before use, an aliquot was thawed for 10′ in a 37° C.-water bath and kept at room temperature for 30′. The plasma was mixed by swirling and/or by resuspending with a pipette; vortexing was avoided, as was done with the IgIV preparations and all other protein solutions used.

For ELISA's Microlon high-binding plates (Greiner Bio-One GmbH, Frickenhausen, Germany; catalogue number 655092, lot 05130103, exp. March 2009) were used. Antibodies used were goat anti-human IgG-alkaline phosphatase (Biosource Int., Camarillo, Calif., USA; catalogue number AHI0305, lot 7602), goat anti-human IgM-alkaline phosphatase (Biosource Int.; catalogue number AHI0605, lot 3903), peroxidase-conjugated rabbit anti-mouse immunoglobulins (RAMPO, catalogue number P0260, DAKOCytomation, Glostrup, Denmark), peroxidase-coupled swine anti-rabbit immunoglobulins (SWARPO, catalogue number P0217, DAKOCytomation), rabbit polyclonal anti-human albumin antibody A-0001 (DAKOCytomation), rabbit polyclonal anti-human haemoglobin antibody A-0118 (DAKOCytomation; lot 122(021)), mouse monoclonal anti-human amyloid-β antibody M0872 (DAKOCytomation; clone 6F/3D, lot 00003503, exp. August 2006), rabbit polyclonal anti-human fibrinogen antibody A0080 (DAKOCytomation; lot 097(701), exp. August 2006) and murine monoclonal hybridoma anti-glucose-6-phosphate glycated human fibronectin antibody 4B5 (lot 2, code 100901BB, ref. (Bouma et al., 2003)). In ELISA's binding of alkaline phosphatase conjugated antibodies was assessed using p-nitrophenyl phosphate disodium 6*H₂O (Sigma-Aldrich, St. Louis, Mo., USA; Phosphatase substrate catalogue number 104, lot 120K6008), and binding of peroxidase-conjugated antibodies was assessed using 1,2-phenylenediamine (‘OPD’, Merck, Darmstadt, Germany; catalogue number 1.07243.0050, lot L937543-844).

Inhibition studies using an ELISA set-up were performed using concentration series of Congo red (Aldrich, Milwaukee, Wis., USA; catalogue number 86,095-6), Thioflavin T (Sigma, St. Louis, Mo., USA; catalogue number T3516, lot 80K3444), Thioflavin S (Sigma; catalogue number T1892), tissue-type plasminogen activator (tPA, Actilyse, Boehringer-Ingelheim, Alkmaar, The Netherlands), or a truncated form of tPA (K2P tPA, Rapilysin, Boehringer-Ingelheim, Alkmaar, The Netherlands) lacking three amino-terminal domains including the fibronectin type I domain, or alternatively designated as finger (F) domain.

Antigens used in IgIV binding ELISA's were synthetic human fibrin peptide 148-KRLEVDIDIGIRS-160 (SEQ-ID 1), with a K157G mutation, synthetic human amyloid-β peptide 1-DAEFRHDSGYEVHHQKLVFFAEDVGSNKGAIIGLMVGGVV-40 (SEQ-ID 2) (Aβ(1-40), synthetic human Aβ(1-40)E22Q Dutch type 1-DAEFRHDSGYEVHHQKLVFFAQDVGSNKGAIIGLMVGGVV-40 (SEQ-ID 3) (Peptide facility, Dutch Cancer Institute, Amsterdam, the Netherlands), bovine serum albumin (BSA, fraction V, catalogue number A-7906, initial fractionation by heat shock, purity ≧98% (electrophoresis), remainder mostly globulins, Sigma-Aldrich, St. Louis, Mo., USA), human haemoglobin (Hb, Sigma-Aldrich; catalogue number H7379), and their advanced glycated endproducts-modified counterparts BSA-AGE and Hb-AGE (see below).

### Methods

### Glycation of Proteins

Glycation of albumin and Hb was performed as follows. For preparation of BSA-AGE, 100 mg ml−¹ of albumin was incubated with phosphate-buffered saline (PBS, 140 mM sodium chloride, 2.7 mM potassium chloride, 10 mM disodium hydrogen phosphate, 1.8 mM potassium di-hydrogen phosphate, pH 7.3) containing 1 M of D-glucose-6-phosphate disodium salt hydrate (anhydrous) (g6p, ICN, Aurora, Ohio, USA) and 0.05% m/v NaN₃, at 37° C. in the dark. The solution was glycated for 70 weeks. Human Hb at 10 mg/ml was incubated for 75 weeks at 37° C. with PBS containing 1 M of g6p and 0.05% m/v of NaN₃. After incubations, albumin and Hb solutions were extensively dialysed against distilled water and, subsequently, aliquoted and stored at −20° C. Protein concentrations were determined with Advanced protein-assay reagent ADV01 (Cytoskeleton, Denver, Colo., USA).

### Preparation of Heat-Denatured Proteins

Heat denatured misfolded proteins were prepared as follows. One mg/ml of Endostatin (recombinantly produced collagen XVIII fragment, EntreMed, Inc., Rockville, Md.; solution), BSA (Sigma-Aldrich; lyophilized, catalogue number A7906), murine serum albumin (MSA, Calbiochem, EMD Biosciences, Inc., San Diego, Calif.; lyophilized, catalogue number 126674), hen egg-white lysozyme (ICN, Irvine, Calif., USA; lyophilized, catalogue number 100831), human glucagon (Glucagen, Novo Nordisk, Copenhagen, Denmark; lyophilized, catalogue number PW60126), purified chicken ovalbumin (OVA, Sigma; catalogue number A7641, lot 071k7094) or human β2-glycoprotein I (β2gpi, purified in-house, from fresh plasma, ref. (Horbach et al., 1996)) in 67 mM NaPᵢ buffer pH 7.0, 100 mM NaCl, was heated for five cycles in PCR cups in a PTC-200 thermal cycler (MJ Research, Inc., Waltham, Mass., USA). In each cycle, proteins were heated from 30 to 85° C. at a rate of 5° C./min. In addition, Endostatin, MSA, ovalbumin and lysozyme were heat-denatured at 1 mg/ml in a similar way, using only one heat incubation cycle. Endostatin at 7.9 mg/ml was diluted in H₂O to 1 mg/ml, MSA and ovalbumin at 1 mg/ml were in PBS pH 7.4, lysozyme was dissolved in PBS with 10 μM HCl added, 1 mg/ml concentration. Control proteins are not subjected to the thermal cycling procedure. To confirm misfolding of the proteins into amyloid-like structures, enhancement of Thioflavin T (ThT) was assessed with heat-treated proteins as well as with control proteins. Fluorescence of ThT-amyloid-like protein/peptide adducts was measured as follows. Solutions of 25 μg ml⁻¹ of protein or peptide preparations were prepared in 50 mM glycine buffer pH 9.0 with 25 μM ThT. Fluorescence was measured at 485 nm upon excitation at 435 nm. Background signals from buffer, buffer with ThT and protein/peptide solution without ThT were subtracted from corresponding measurements with protein solution incubated with ThT. Regularly, fluorescence of Aβ was used as a positive control, and fluorescence of synthetic human fibrin fragment FP10 (148-KRLEVDIDIK-157 (SEQ-ID 4); Peptide facility, Dutch Cancer Institute, Amsterdam, the Netherlands), a non-amyloid fibrin fragment (Kranenburg et al., 2002), and buffer was used as a negative control. Fluorescence was measured in triplicate on a Hitachi F-4500 fluorescence spectrophotometer (Hitachi, Ltd., Tokyo, Japan). Alternatively, Congo red fluorescence was analyzed in a similar way. Now, excitation and emission wavelengths were 550 and 590 nm. Again, 25 μg/ml of tester proteins was analyzed, in 25 μM Congo red solutions.

Alternatively, a heat denatured amyloid peptide was prepared as follows. Human fibrin peptide NH₂-IDIKIR-COOH (SEQ-ID 6, FP6) was dissolved at approximately 10 mg/ml in a 1:1 volume ratio of 1,1,1,3,3,3-hexafluoro-2-propanol and trifluoro acetic acid. The organic solvents were vaporized under an air stream. FP6 was dissolved in distilled water to a final concentration of 1 mg/ml and kept at 37° C. for 72 h. The solution was subsequently stored at room temperature. Presence of crossbeta structure conformation was confirmed by measuring enhancement of fluorescence of amyloid specific dyes ThT and Congo red and by X-ray fiber diffraction analysis (personal communication, L. Kroon-Batenburg, Bijvoet Center for Biomolecular Research, Dept. of Crystal & Structural Chemistry, University of Utrecht, The Netherlands) (data not shown here). Furthermore, the property of FP6 solutions to activate tPA in a plasminogen/plasmin/chromogenic substrate conversion assay was assessed and found to be positive (data shown elsewhere).

Preparation of Yeast Prion Peptide Oligomers with Amyloid-Like Conformation

The peptide fragment NH₂-GNNQQNY-COOH of the yeast prion protein (SEQ-ID 5) was purchased from the Peptide Facility of the Netherlands Cancer Institute (H. Hilkmann, NKI-Amsterdam, The Netherlands; lot 5LKB1-2081). Purity of the peptide was analyzed by performing reversed phase HPLC and was ˜90%. The peptide was dissolved to final concentrations of 1 and 10 mg/ml in H₂O. The clear solutions were incubated for 72 h at 4° C. at a rollerbank or for 5 h at room temperature without motion. Enhancement of Congo red fluorescence was determined as a measure for the presence of amyloid like conformation (see above). In addition, formation of crossbeta structure with this batch of peptide was confirmed with X-ray fiber diffraction analysis using a solution of 10 mg/ml in H₂O (personal communication, L. Kroon-Batenburg, Bijvoet Center for Biomolecular Research, Dept. of Crystal & Structural Chemistry, University of Utrecht, The Netherlands) (data not shown here).

### Preparation of Oxidized Proteins

Oxidation of proteins was performed using prolonged exposure of proteins in solution to CuSO₄. Proteins used were human normal pooled citrated plasma of apparently healthy persons, formulated Endostatin (EntreMed, Inc., Rockville, Md.; 7.9 mg/ml solution), chicken egg-white lysozyme (ICN, catalogue number 100831, lot 98032), human haemoglobin (Sigma-Aldrich, catalogue number H7379, lot 039H7605), human glucagon (Glucagen from NovoNordisk Farma B.V., lot RW 60038), bovine albumin (Sigma-Aldrich, A7906, lot 81K1813), human γ-globulins (Sigma-Aldrich, G4386, lot 21K7600), chicken egg-white ovalbumin (Sigma-Aldrich, A7641, lot 071K7094). Lyophilized proteins were dissolved at 2 mg/ml in PBS, plasma was 40 times diluted and Endostatin was diluted to 2 mg/ml in PBS. NaN₃ stock solution of 2% m/v was added to a final concentration of 0.02%. CuSO₄ stock solution of 1 M in H₂O was added to a final concentration of 10 mM. In control protein solutions H₂O was added instead of CuSO₄. All protein solutions were mixed by swirling, avoiding vortexing. Solutions were kept at 4° C. on a rollerbank for 72 h. Enhancement of ThT was measured (see above).

Alternatively, proteins were oxidized by introducing 10 μM CuSO₄ in the solutions. In this way, ovalbumin, albumin, endostatin, lysozyme, γ-globulins all at 2.5 mg/ml and glucagon at 1 mg/ml were incubated for 144 h at 37° C. in PBS. In control protein solutions, CuSO₄ was omitted. Thioflavin T fluorescence was measured as a measure for the presence of misfolded proteins with crossbeta structure conformation. Protein solutions that showed enhanced ThT fluorescence were dialyzed against PBS, as well as their non-oxidized controls.

Low density lipoproteins (LDL) were isolated from fresh (<24 h) human plasma that was kept at 10° C., obtained from the Netherlands bloodbank. LDL was isolated essentially as earlier described (4). Plasma was centrifuged in an ultracentrifuge for three subsequent cycles. The LDL fraction was isolated and stored under N₂, at 4° C. Before experiments, native LDL (nLDL) was dialyzed overnight at 4° C. against 0.9% w/v NaCl. To obtain oxidized LDL (oxLDL) with varying degrees of oxidation, native LDL was first dialyzed against 0.15 M NaCl solution containing 1 mM NaNO₃, overnight at 4° C. Then, nLDL was diluted to 3-5 mg/ml, and CuSO₄ was added to a final concentration of 25 μM and incubated at 37° C. In a similar way LDL was oxidized using FeSO₄ instead of CuSO₄. Oxidation with FeSO₄ was also preceded by the dialysis step. Next, LDL was dialyzed against 5 μM FeSO₄ in PBS with additional 150 mM NaCl and 1 mM NaN₃, pH 7.2. The degree of oxidation is controlled by choosing a certain number of oxidation buffer refresh cycles. The more often FeSO₄ in buffer is refreshed each 10-12 h, the higher the degree of oxidation will be. To stop oxidation, the LDL sample is dialyzed against a buffer of 150 mM NaCl, 1 mM NaN₃, 1 mM EDTA for 4 h at 4° C. The degree of oxidation was followed by measurement of diene-formation at λ=234 nm (Ultrospec 3000 Spectrophotometer (Pharmacia Biotech)). To stop the oxidation reaction, LDL was dialyzed against 0.15 M NaCl, 1 mM NaNO₃ and 1 mM EDTA. LDL solutions were stored at 4° C. under N₂. Presence of crossbeta structure conformation in the ApoB protein portion of LDL was analyzed using a Thioflavin T fluorescence assay (see above).

### Preparation of Misfolded Proteins Using Denaturing Surfaces

To prepare misfolded proteins upon exposure to surfaces composed of multimeric molecules, CpG-ODN (Coley Pharmaceutical Group, MA, USA) at 21.4 μg/ml or lipopolysaccharide (LPS, from Escherichia coli serotype 011:B4, #L2630, lot 104K4109, Sigma-Aldrich) at 600 μg/ml were mixed with 1 mg/ml of chicken egg-white lysozyme (lyophilized, Fluka, Sigma-Aldrich; catalogue number 62971), BSA, Endostatin and ovalbumin, and incubated o/n at 4° C., or for 1 h at room temperature, on a roller bank. For this purpose, lyophilized proteins were dissolved in HEPES-buffered saline (HBS, 10 mM HEPES, 4 mM KCl, 137 mM NaCl, pH 7.2) to a final concentration of 2 mg/ml, and Endostatin at 7.9 mg/ml was diluted to 2 mg/ml in HBS. Proteins were gently dissolved on a roller bank at room temperature for 10 min, at 37° C. and at room temperature for 10 min. The protein solutions at 2 mg/ml were then ultracentrifuged for 1 h at 100,000*g before use, and subsequently diluted 1:1 in HBS with 42.9 μg/ml CpG-ODN or with 1200 μg/ml LPS. Formation of amyloid-like crossbeta structure was assessed by measuring enhancement of Thioflavin T fluorescence with respect to control protein solutions in which the denaturing surfaces was omitted. For this purpose, proteins were diluted to 25 μg/ml and incubated with assay buffer or with 25 μM Thioflavin T in assay buffer (see above for assay details).

Alternatively, misfolded proteins are obtained after exposure of proteins to denaturing molecules such as (negatively charged) (phospho)lipids such as phosphatidyl serine and cardiolipin, dextran sulphate (500,000 Da), alum, ellagic acid, glass or kaolin. These misfolded proteins are included in tests conducted to reveal the working mechanism of IgIV action.

### Enzyme Linked Immunosorbent Assay for Testing of IgIV Binding to Misfolded Proteins

Binding of IgIV or of immunoglobulins in normal pooled plasma was determined using an enzyme linked immuno sorbent assay (ELISA) set-up. For this purpose 50 μl/well of potential ligands at indicated concentrations or coat buffer only for control and background measurement purposes, were coated overnight at 4° C., with motion, in 50 mM NaHCO₃ pH 9.6. Glycated albumin and Hb (BSA-AGE and Hb-AGE), control BSA and control Hb were coated at 5 μg/ml. Aβ and FP13 were coated at 25 μg/ml. The BSA and Hb controls were prepared freshly by dissolving lyophilized proteins at 1 mg/ml in PBS upon resuspending by pipetting, followed by a 30′ period at the roller bank, at room temperature. The protein solutions were centrifuged for 10′ at 16,000*g and diluted in coat buffer. Coat controls were performed with anti-glycated protein antibody, anti-albumin antibody, anti-Hb antibody and anti-Aβ antibody. FP13 was not recognized by a polyclonal anti-fibrinogen antibody. The alkaline phosphatase-conjugated anti-human Ig antibodies were controlled by coating the IgIV's and overlaying them with the secondary antibodies. After coating the plates were washed twice with 50 mM Tris-HCl pH 7.3, 150 mM NaCl, 0.1% v/v Tween20, and blocked with 175 μl/well Blocking reagent (Roche Diagnostics, Almere, The Netherlands; catalogue number 11112589001), for 1 h at room temperature, with motion. Plates were washed twice and incubated in triplicate with indicated antibodies dilution series, plasma dilution series or controls, including binding buffer only, in the absence or presence of putative inhibitors, in binding buffer; PBS/0.1% v/v Tween20, at 50 μl/well, for 1 h at room temperature, with constant motion. After four wash cycles, secondary antibodies were added to the wells, 50 μl/well, for 45′ at room temperature, with motion. RAMPO and SWARPO were used at 2000 times dilution, goat anti-human IgG antibodies were diluted 3000 times, goat anti-human IgM antibodies were diluted 1000 times. After 5 washes with wash buffer followed by two washes with PBS, binding of antibodies was assessed. For alkaline phosphatase conjugated secondary antibodies p-nitrophenyl phosphate (600 μg/ml) in DEA buffer pH 9.8 (10% v/v diethanolamine in H₂O, with 240 μM MgCl₂.6H₂O, pH adjusted with HCl) was used at 100 μl/well, for ˜5′. The reaction was stopped by adding 50 μl/well of 2.4 M NaOH in H₂O. After 5′ absorbance was read at 405 nm. For peroxidase-conjugated RAMPO and SWARPO, OPD (1.3 mg/ml) in 50 mM citric acid/100 mM Na₂HPO₄/0.06% v/v H₂O₂ pH 5 was used at 100 μl/well, for ˜5′. The reaction was stopped by adding 50 μl/well of 2 M H₂SO₄ in H₂O. After 5′ absorbance was read at 490 nm. Each experiment has been performed at least twice. To test whether amyloid-like crossbeta structure binding compounds and controls (see ref. (Bouma et al., 2003) and patent application P57716EP00) interfere with IgIV binding to crossbeta structure ligands, concentration series of the potential inhibitors were tested in the presence of a suboptimal IgIV concentration. For this purpose stock solutions used of tPA, K₂P tPA, Congo red, Thioflavin S (ThS) and Thioflavin T (ThT) were 3.7 mg/ml, 1.1 mg/ml, 10 mM, 10 mM and 10 mM, respectively. The influence of tPA and K2P tPA was tested in the presence of 10 mM ε-amino caproic acid, to avoid binding of the kringle2 domain of tPA and K2P tPA to lysine- and arginine residues (tPA binding to amyloid-like structures is mediated by its finger domain, that is lacking in truncated K2P tPA; the kringle2 domain binds to exposed side chains of lysines and arginines). Binding buffer and K2P tPA serve as negative controls in these inhibition studies. Separately, similar inhibition studies were performed with immobilized Aβ or BSA-AGE, a suboptimal concentration of tPA (see ref (Bouma et al., 2003; Kranenburg et al., 2002)) and concentration series of Congo red or ThT. Data reduction was performed as follows. Triplicates were averaged and standard deviations calculated. Background signals obtained with buffer-coated wells were subtracted (binding of primary antibody to empty wells), as well as background signals obtained with wells in which the primary antibodies were omitted (binding of secondary antibody to coated ligands).

In a separate series of experiments yeast prion peptide NH₂-GNNQQNY-COOH (SEQ-ID 5) was coated to the ELISA plates at a concentration of 25 ˜g/ml. The stock solutions of 1 mg/ml that was incubated at 4° C. for 73 h was used. In control wells, 5 μg/ml Hb-AGE or coat buffer was coated. Binding of a dilutions series IgIV (I) was analyzed and compared to binding of concentration series of tPA and K2P tPA. In addition, a mixture of five monoclonal antibodies which have affinity for misfolded proteins, was also tested for binding to the immobilized ligands (see below for monoclonal details). For this purpose, a mixture comprising 336 μg/ml of each of the five antibodies was prepared in PBS, resulting in a stock solution of 1.83 mg/ml total antibody.

### Preparation of Murine Monoclonal Anti-Misfolded Proteins Antibodies

The immunizations were performed by the ABC-Hybridoma facility (P. van Kooten & M. Smits, Utrecht University, The Netherlands). A mouse (Balb/c) was immunized with 100 μg Aβ in 100 μl H₂O and 100 μl complete Freund's adjuvant. After three weeks, a first boost of 50 μg Aβ in H₂O-Specol (ID-DLO, Lelystad, The Netherlands) was given, followed by a second boost 30 days after the first boost. Thirty-six and 37 days after the second boost, the mouse was given two additional boosts with 50 μg Aβ in PBS (intravenously). Between approximately week 44 and week 48 after the start of the immunization with Aβ, the mouse got ill, but recovered. Forty nine weeks later, the mouse was immunized with 50 μg recombinant chicken serum amyloid A in H₂O-Specol. This antigen was a kind gift of Dr H. Toussaint (Dept. of Veterinary Medicine, University of Utrecht, The Netherlands). Four weeks later, the mouse was immunized with 50 μg Hb-AGE. Finally, 31 and 32 days later the mouse was boosted twice intravenously with 50 μg FP6 (SEQ-ID 6) in PBS. Three days after the final boost, the mouse was sacrificed and the spleen was used to prepare hybridomas. Fusion medium was enriched with PEG4000 (Merck, catalogue number 9727). The spleen comprised an exceptionally high number of cells, i.e. 7*10⁸ cells, with a relatively high abundance of infiltrated fibroblasts. 2*10⁸ cells were mixed with 4*10⁷ Sp2/0 plasmacytoma cells for the fusion. After fusion selective hybridoma culture medium consisting of OptiMEM I with 10% Fetalclone1 (Hyclone), 4 μM Aminopterin and 1% Glutamax I was used. After an incubation time to allow for fusion of the spleen β-cells and the plasmacytoma cells, cells were transferred at 1 cell per well to 96-wells plates, using a FacsVantage apparatus with Accudrop software. After approximately two weeks hybridomas were screened for putative production of anti-cross-β structure antibodies. First, 768 clones in 96-wells plates were screened for the presence of antibodies that bind to immobilized FP13 K157G amyloid and amyloid γ-globulins. For this purpose, FP13 K157G and amyloid γ-globulins were diluted together in H₂O to 5 μg ml⁻¹ of each polypeptide. Microlon high-binding ELISA plates (Greiner, Bio-One GmbH, Frickenhausen, Germany) were filled with 50 μl of this solution and air-dried overnight at 37° C. Plates were blocked with Blocking reagent (catalogue #11112589001, Roche Applied Science, Basel, Switzerland) and washed with tap water. One hundred μl of hybridoma cell culture supernatants containing 10% v/v fetal calf serum was transferred to the coated plates and incubated for 1 h at room temperature (RT) while shaking. Plates were washed with Tris-buffered saline pH 7.3 (TBS, 50 mM Tris-HCl, 150 mM NaCl) with 0.1% Tween-20 (wash buffer), and subsequently overlayed with 2000× diluted peroxidase-coupled rabbit anti-mouse immunoglobulins (RAMPO, #P0260, DAKO, Denmark) in PBS/0.1% Tween-20, for 30′ at RT while shaking. After extensive washing, bound RAMPO was visualized with tetramethylbenzidine (TMB, #45.01.20, /#45.014.01, Biosource, Nivelles, Belgium). The reaction was stopped after 5 minutes with 1% H₂SO₄ in H₂O. Plates were read at 450 nm. Clones were included in further screening trials when signals reached at least 1.5× background levels. Again, presence of putative anti-cross-β structure antibodies was analyzed with immobilized FP13 K157G and amyloid γ-globulins. Then, 35 clones remained positive. Those clones were transferred to cell culture flasks and subjected to further analyses. For this purpose, again FP13 K157G and amyloid γ-globulins, now separately, as well as Aβ and Hb-AGE were immobilized on ELISA plates. In addition, freshly dissolved Aβ, FP13 K157G, Hb and γ-globulins were coated onto Immobilizer plates (Exiqon, Vedbæk, Denmark). These freshly dissolved controls were coated at 20, 12.5, 50 and 50 μg ml-1, respectively, in PBS, for 1 h at RT while shaking. Aβ, FP13 K157G, Hb and γ-globulins stock solutions of 20, 12.5, 50 and 50 μg ml⁻¹, respectively, were first centrifuged for 30 min. at 238*10³×g to remove insoluble aggregates that might be present. Buffer was coated on Greiner (H₂O) and on Exiqon (PBS) plates as additional negative control. Greiner plates were not blocked during initial screens with 768 clones. Ten % FCS in the cell culture medium is an efficient blocker during the incubation of cell supernatant in the ELISA plates. Ten μl of PBS/1% Tween-20 was added to the wells of the Exiqon plates, before cell supernatants were added. Tween-20 at a concentration of 0.1% is an effective instant blocker for Immobilizer plates. Hundred μl of the hybridoma supernatants was transferred to the plates. Culture medium was used as negative control. Signals were calculated as multiples of the signals obtained when fresh culture medium with 10% FCS was incubated on the various immobilized antigens and controls. Signals were considered positive when exceeding 2.0× the background values obtained with fresh culture medium. Subsequent screening of 21 out of 35 clones was performed on Greiner plates, prepared as described above. The plates were now first blocked with Blocking reagent and washed. Fifty μl of each hybridoma clone supernatant was tested in duplicate for the presence of sequence independent, but structure specific antibodies, fresh culture medium was tested in fourfold as control. From the original 21 clones, six were selected for further single cell sub-cloning to obtain monoclonal hybridomas. The six clones were seeded at one cell per well of a 96-wells culture plate and cultured in medium enriched with 10% v/v FCS. The clones were all tested for binding to two coated amyloids. For each of the six clones five sub-clones were identified that bound to the two amyloids, for subsequent culturing in 25 cm² culture flasks. Isotyping of the thirty subclones using fluorescently labeled isotype-specific antibodies has been performed by the ABC-Hybridoma facility (M. Smits) according to the recommendations of the manufacturer (Luminex, Austin, Tex., USA). The antibodies were purified from cell culture medium using conventional chromatographic purification technology. Samples were subjected to thiophillic chromatography using AFFI-T gel matrix (KemEnTEC, Biozym, Landgraaf, The Netherlands) in an Econo column (Biorad, Veenendaal, The Netherlands). Purified antibodies were stored at −20° C. in PBS.

In order to obtain monoclonal antibodies, a mouse was sequentially immunized with human amyloid Aβ(1-40) E22Q, recombinant chicken serum amyloid A and glycated human haemoglobin with amyloid-like properties, followed by a final boost with amyloid human fibrin peptide FP6. Hybridomas were formed and their cell culture supernatants were screened for the presence of antibodies that specifically recognize an epitope that is only recognized when cross-β structure conformation is present in any polypeptide with an amino-acid composition that is unrelated to antigens used for immunization. Out of 768 clones six clones, 2E2, 4F4, 7H1, 7H2, 7H9 and 8F2, were selected that show affinity for a broader range of amyloid-like aggregates other than the antigens used for immunization. After several rounds of selection and subcloning finally the following five monoclonal antibodies showed consistent binding to misfolded proteins with crossbeta structure conformation: 2E2B3D12, 7H2H2, 7H1C6A7, 7H₉B9, 8F2G7H7. The 7H2H2 clone specifically binds only to various misfolded forms of immunoglobulins, which let us to type this clone as a ‘Rheuma factor-like antibody’. A mixture of the five listed monoclonal antibodies was prepared in which the final concentrations of the individual antibodies was 1.5, 0.37, 0.4, 0.45 and 0.47 mg/ml for 2E2B3D12, 7H2H2, 7H1C6A7, 7H₉B9 and 8F2G7H7, respectively, giving an overall antibody concentration of 3.2 mg/ml. Alternatively, all antibodies were diluted in PBS to 1.83 mg/ml and combined 1:1:1:1:1 resulting in a total antibody concentration of 1.83 mg/ml with 336 μg/ml of the individual antibodies. These mixtures of murine anti-misfolded protein antibodies were used as stock solutions for further blood platelet aggregation assays (see Example).

### Platelet Aggregation

The influence of IgIV on blood platelet aggregation induced by aggregates of misfolded glycated Hb with amyloid-like crossbeta structure conformation was tested with washed platelets in an aggregometric assay. Freshly drawn human aspirin free blood was mixed gently with citrate buffer to avoid coagulation. Blood was spinned for 15′ at 150*g at 20° C. and supernatant was collected; platelet rich plasma (PRP). Buffer with 2.5% trisodium citrate, 1.5% citric acid and 2% glucose, pH 6.5 was added to a final volume ratio of 1:10 (buffer-PRP). After spinning down the platelets upon centrifugation for 15′ at 330*g at 20° C., the pellet was resuspended in HEPES-Tyrode buffer pH 6.5. Prostacyclin was added to a final concentration of 10 ng/ml, and the solution was centrifuged for 15′ at 330*g at 20° C., with a soft brake. The pellet was resuspended in HEPES-Tyrode buffer pH 7.2 in a way that the final platelet number was adjusted to 200,000-250,000 platelets/μl. Platelets were kept at 37° C. for at least 30′, before use in the assays, to ensure that they were in the resting state. Platelets of five donors were isolated separately on three different days (2, 2, 1).

For the aggregometric assays, 270, 280 or 300 μl platelet solution was added to a glass tube and prewarmed to 37° C. A stirring magnet was added and rotation was set to 900 rpm, and the apparatus (Whole-blood aggregometer, Chrono-log, Havertown, Pa., USA) was blanked. A final volume of 30, 30 or 33.3 μl was added, containing the agonist of interest and/or the premixed antagonist of interest, prediluted in HEPES-Tyrode buffer pH 7.2. Aggregation was followed in time by measuring the absorbance of the solution, that will decrease in time upon platelet aggregation. As a positive control, either 10 μg/ml collagen (Kollagenreagens Horm, NYCOMED Pharma GmbH, Linz, Austria; lot 502940), or 5 μM of synthetic thrombin receptor activating peptide TRAP (NH₂-SFLLRN-COOH, SEQ-ID 7) was used. Aggregation was recorded for 15′ and expressed as the percentage of the transmitted light (0-100%).

Preparation of a Crossbeta Structure Affinity Matrix for Capturing Proteins that Bind to Misfolded Proteins

In order to be able to further investigate whether a subset of the Ig's in IgIV binds to misfolded proteins, we prepared an affinity matrix with linked misfolded protein. For this purpose, we coupled glycated Hb to CNBr-Sepharose (GE Healthcare-Amersham, Roosendaal, The Netherlands) according to the manufacturer's recommendations. For overnight coupling at 4° C. at a rollerbank, 250 μg of HCl-washed and coupling-buffer washed, aspirated beads (dry weight) was incubated with 125 μl coupling buffer only (control beads) (100 mM NaHCO₃, pH 8.3, 500 mM NaCl) or with coupling buffer with 3.33 mg/ml Hb-AGE. After extensive washing, we determined whether Hb-AGE was coupled to the Sepharose and whether the prepared affinity matrix was indeed capable of capturing proteins that have affinity for misfolded proteins with crossbeta structure conformation. Coupling efficiency was determined by comparing the concentration of Hb-AGE starting material with the Hb-AGE supernatant after the coupling reaction. Dilution series were prepared in ADV01 protein stain (Cytoskeleton) and absorbance was read at 590 nm. Comparing absorbance signals revealed that 50% of the Hb-AGE was bound to the Sepharose, i.e. approximately 200 μg Hb-AGE at 250 μg beads (dry weight).

Previously, we established that tissue-type plasminogen activator is an enzyme with affinity for misfolded proteins with crossbeta structure conformation, including glycated proteins (Bouma et al., 2003; Kranenburg et al., 2002). To test the ability of the Hb-AGE-affinity matrix to bind tPA, twenty μl of a 1:1 suspension of Hb-AGE Sepharose or control Sepharose in HBS was incubated with 6 μM tPA (optimized concentration after testing 0-10 μM concentration series) by overnight incubation at 4° C. at a rollerbank, in duplicates. After two minutes centrifugation at 8,000*g and discarding the supernatant, beads were washed five times with HBS. Bound tPA was eluted by incubating the matrix for 1 h at room temperature with 20 μl elution buffer (10 mM HEPES pH 7.4, 1140 mM NaCl, 10 mM ε-amino caproic acid, 4.5 mM CaCl₂, 0.005% Tween20). The eluate was analyzed for the tPA content and this was compared with the tPA content of the incubation mixture before and after contacting the Hb-AGE Sepharose or control Sepharose. Relative tPA concentrations were determined using a chromogenic tPA substrate S2765 (Chromogenix, Instrumentation Laboratory SpA, Milano, Italy). For this purpose, 1-5 μl of tester samples (tPA starting solutions, supernatant after contacting the Hb-AGE Sepharose, eluate after incubation of Hb-AGE Sepharose with elution buffer) was mixed with 10 μl 5 mM S2765 and 5 μl of a 10 times HBS stock solution, and adjusted with H₂O to a final volume of 50 μl. Conversion of the substrate by tPA from a colourless agent to a yellow substance was recorded in time at an absorbance 96-wells kinetic plate reader, at 37° C.

Next, 120 μl or 20 μl of the affinity Hb-AGE Sepharose matrix or 120 μl or 20 μl of the control Sepharose without coupled protein were incubated with 200 μl of the 50 mg/ml IgIV (Octagam) stock solution (4 h at room temperature). Then, the concentration of IgIV remaining in solution and the amount of bound IgIV to the matrix after extensive washing with incubation buffer, was determined. Protein concentrations were determined by measuring absorbance at 280 nm, using an IgIV standard dilution series, and by comparing absorbance at 590 nm of IgIV samples after staining with ADV01 (Cytoskeleton), with staining of an IgIV standard dilution series. IgIV bound to Hb-AGE-Sepharose or to control-Sepharose was washed six times with approximately two volumes of HBS (binding buffer). Then, bound IgIV was eluted with 200 μl HBS with 1 M NaCl and 10 mM ε-amino caproic acid (30′ at room temperature, with agitation). Binding to Hb-AGE immobilized on an ELISA plate was analyzed with dilution series of untreated IgIV, IgIV after contacting Hb-AGE-Sepharose, IgIV after contacting control-Sepharose, eluted IgIV from Hb-AG-Sepharose, eluted IgIV from control-Sepharose. For this purpose, 5 μg/ml Hb-AGE or heat-denatured BSA was coated for 1 h at room temperature, with agitation (Greiner Microlon high-binding plate). Plates were blocked with Blocking Reagent (Roche). Binding of dilutions series of the IgIV preparations was assessed as described above. The relative amount of IgIV in eluted IgIV from Hb-AGE-Sepharose and in eluted IgIV from control-Sepharose was calculated with respect to the IgIV stock. For this purpose a standard curve was prepared of a dilution series of IgIV bound to Hb-AGE or bound to heat-denatured BSA. Enrichment of the eluted IgIV from Hb-AGE-Sepharose with respect to binding to coated Hb-AGE was assessed using IgIV that was incubated with 120 μl Sepharose. Enrichment with respect to binding to heat-denatured BSA was assessed with IgIV incubated with 20 μl Sepharose.

Immunohistochemical Stain of Brain Sections of Deceased Creutzfeldt-Jakob's Disease Patients with IgIV and a Mixture of Monoclonal Anti-Misfolded Protein Antibodies

For immunohistochemical stains of brain sections of deceased Creutzfeldt-Jakob's disease (CJD) patients with sporadic CJD or new variant CJD, paraffin sections were prepared (Dept. of Pathology, University Medical Center Utrecht, The Netherlands). The sections were applied to a standard stain procedure comprising the following steps: 1. fixed sections were blocked with block buffer, 2. incubated with IgIV or monoclonal antibodies with affinity for misfolded proteins with crossbeta structure conformation, diluted in binding buffer, 3. washed, 4. incubated with an anti-human IgG antibody and anti-murine IgG/IgM antibodies, respectively, 5. washed, 6. incubated with Powervision, 7. washed, 8. stained with DAB, and 9. enclosed and mounted, decontaminated by an acid treatment, before microscopic analysis and scoring. The procedure was performed by qualified personel in the authorized category III laboratory equipped for working with TSE-contaminated materials, located in the UMC Utrecht, The Netherlands). As control sections were incubated consecutively with tPA, a murine monoclonal anti-tPA antibody and Powervision, followed by DAB stain. As a control for the stain procedure, brain sections of a deceased Alzheimer's disease patient were also incubated with IgIV or tPA, following the same procedure as given above.

### Results & Discussion

### Example 1

### IgIV (Human Immunoglobulin IgG Antibodies) Bind to Misfolded Proteins Comprising Crossbeta Structure Conformation

Non-enzymatic modification of proteins by carbohydrates, a process termed glycation induces protein misfolding accompanied with formation of amyloid crossbeta structure (Bouma et al., 2003). Binding of IgIV to immobilized glycated proteins Hb-AGE and BSA-AGE and non-glycated Hb and BSA was established using an ELISA set-up (FIG. 1A-C). Binding of IgIV was detected using alkaline-phosphatase-labeled anti-human IgG or IgM antibodies. Both IgIV (I) and IgIV (II) bound with high affinity to glycated proteins comprising crossbeta structure, whereas they bound weakly to immobilized native albumin and native haemoglobin (FIG. 1A-C). Affinity of IgIV (I) for immobilized protein was higher than of IgIV (II). Affinity of IgIV (I) for Hb-AGE was higher than for BSA-AGE. Depending on the albumin or haemoglobin preparation, a slightly varying amount of IgIV bound to these ‘native’ proteins, most likely due to varying amounts of molecules with a non-native conformation, exposing the binding site for IgIV antibodies with affinity for misfolded proteins.

Tissue-type plasminogen activator is a serine protease containing a module, termed the finger domain, that specifically interacts with misfolded proteins comprising crossbeta structure (Kranenburg et al., 2002; Gebbink et al., 2005). Binding of IgIV (I) at the suboptimal concentration of 15 μg/ml to coated glycated proteins is effectively diminished by a concentration series of tPA, whereas truncated K2P tPA has no influence on IgIV (I) binding (FIG. 1D). It is known that tPA binds with relatively high affinity (kD of approximately 500 μM) to glycated proteins and, with somewhat lower affinity, to many other misfolded proteins with amyloid-like protein conformation comprising crossbeta structure, most likely via its fibronectin type I domain, which is lacking in K2P tPA. Similar to tPA, also the amyloid-specific dye Congo red effectively blocks the binding of 15 μg/ml IgIV (I) to coated glycated protein (FIG. 4A).

To assess whether IgIV has broad-range specificity for any misfolded proteins, without limitations to the amino-acid sequence of the protein with crossbeta structure, heat-denatured MSA, ovalbumin, and glucagon were analyzed for IgIV binding, as well as oxidized ovalbumin, glucagon, haemoglobin and LDL, and the control non-oxidized or non-heat-denatured counterparts. For this purpose all proteins were immobilized on a Greiner microlon high-binding plate at 25 μg/ml concentration in 50 mM NaHCO₃ (glucagon: 12.5 μg/ml), and overlayed with a concentration series of IgIV (I), 0/1/3/9/27/81/243/729 μg/ml in PBS/0.1% v/v Tween20.

In conclusion, these results demonstrate that IgIV binds to immobilized misfolded proteins that comprise crossbeta structure conformation. To further substantiate these findings, binding to a series of misfolded proteins is assessed. For example, binding of IgIV to oxidized proteins, heat-denatured proteins, proteins denatured upon exposure to (biocompatible) surfaces, e.g. in extracorporal circulation devices, to disease related misfolded proteins (e.g. amyloid-β (Alzheimer's disease); β2-microglobulin (dialysis)), is addressed.

### Example 2

### Blood Platelet Aggregation is Induced by Amyloid-Like Misfolded Protein and is Inhibited by Human IgIV and Murine Monoclonal Antibodies

Platelets isolated from freshly drawn citrated blood of apparently healthy human volunteers readily aggregate when exposed to misfolded glycated proteins, as shown for platelets from three different individuals (donor ‘A’, ‘B’, ‘C’) with Hb-AGE (FIG. 2). When the misfolded protein Hb-AGE or BSA-AGE is pre-incubated with IgIV (I) (FIG. 2A, C) or with a mixture of five monoclonal antibodies (2E2B3D12, 7H2H2, 7H1C6A7, 7H₉B9, 8F2G7H7) with affinity for misfolded proteins comprising crossbeta structure conformation (FIG. 2E, F), platelet aggregation is inhibited. Induction of platelet aggregation by collagen or TRAP, is hardly influenced by the IgIV (I) or mixed monoclonal antibodies (FIG. 2B, D), indicating that the monoclonal antibodies specifically inhibit the effects mediated by proteins comprising crossbeta structure.

In a separate series of experiments using platelets of human donors D and E, platelet aggregation was induced by 50 μg/ml Aβ (FIG. 3). The influence of 2.5 mg/ml IgIV (I) or of the monoclonal antibody mixture on amyloid-induced aggregation was addressed (FIG. 3). Both IgIV (I) and the monoclonal mixture inhibit amyloid-induced platelet aggregation with platelets of two different donors (D and E). Donor D shows a higher % final aggregation than donor E upon stimulation by Aβ. For both donors, IgIV (I) delays the start of platelet aggregation by approximately 2 minutes. Platelets of donor D that are incubated with both Aβ and IgIV finally aggregate to a similar extent when compared to incubation with Aβ. With platelets of donor E, addition of IgIV to Aβ results in a stronger inhibition of platelet aggregation. Four μM TRAP was applied as a positive control. In control experiments the influence of IgIV or monoclonal antibodies on TRAP activation of platelets was analyzed by pre-incubating the TRAP stock with the mixture of monoclonal antibodies. These aggregation experiments showed that the IgIV or the monoclonal antibodies do not influence TRAP induced aggregation (not shown).

These results show that human IgIV contains antibodies that inhibit platelet aggregation induced by glycated proteins and Aβ comprising crossbeta structure. The mixture of monoclonal anti-misfolded protein antibodies exhibit a similar inhibitory activity indicative for the presence of anti-misfolded protein antibodies in the human IgIV therapeutic solution. A wide variety of misfolded proteins are now tested for their ability to induce platelet aggregation. Subsequently the influence of either human IgIV or murine anti-misfolded protein antibodies is addressed to substantiate the current findings. Alternative misfolded proteins used to induce platelet aggregation are, but are not limited to, oxidized proteins, (heat-)denatured proteins, glycated proteins, proteins exposed to denaturing surfaces or denaturing molecules, e.g. CpG-ODN, lipopolysaccharides, dextran sulphate, kaolin, glass, lipids, or amyloid peptides, e.g. FP6, amyloid-β, FP13.

### Example 3

### Potentiation of Binding of IgIV and tPA to Misfolded Protein, by Thioflavin T and Thioflavin S

Two amyloid-specific dyes, Thioflavin T and Thioflavin S, inhibit IgIV-glycated protein interaction to some extent at relatively low dye concentrations, whereas at relatively high dye concentrations both dyes seem to facilitate binding of IgIV to immobilized misfolded protein (FIG. 4B, C). This is explained by the fact that binding of an amyloid-specific dye to a misfolded protein facilitates subsequent binding of a protein with affinity for binding to misfolded proteins. Thioflavin T and Thioflavin S binding stabilizes the surrounding molecules or part of molecules with crossbeta structure conformation in a relatively fixed state that represents a binding site for IgIV. At low dye concentrations, these forces are yet too weak to provoke fixation into a more uniform, IgIV-binding site exposing crossbeta structure. Now, dye binding directly competes for IgIV binding sites. At higher dye concentrations, bound dye molecules exert their stabilizing forces to the surrounding crossbeta structure in concert, thereby creating readily accessible binding sites for IgIV. Similar effects of Congo red and Thioflavin T are seen when binding of a suboptimal concentration of tPA to immobilized BSA-AGE or Aβ is considered (FIG. 4D-G). The observation that binding of an amyloid-specific molecule to crossbeta structure under certain conditions facilitates binding of another molecule with specificity for misfolded proteins is used to improve the efficacy of drugs, such as antibodies, and to treat protein misfolding diseases, such as amyloidosis.

### Example 4

### Misfolded Protein-Sepharose Affinity Matrix for Binding Proteins with Affinity for Ligands with Amyloid-Like Crossbeta Structure Conformation

Immobilization of extensively glucose-6-phosphate glycated haemoglobin, Hb-AGE, to CNBr-Sepharose matrix resulted in an efficient affinity matrix for capturing tPA from solution (FIG. 5). It is shown that tPA specifically binds to the misfolded protein affinity matrix (FIG. 5A). This is further depicted by analysing the tPA content of the wash buffer after incubation of this buffer with tPA-incubated Hb-AGE misfolded protein affinity Sepharose matrix or tPA-incubated control matrix without coupled protein (FIG. 5B). Hardly any tPA serine protease activity is recovered in the wash buffer after washing Hb-AGE Sepharose, and some tPA activity is seen in the wash buffer after washing tPA-incubated control beads. After incubation of tPA-incubated affinity matrix and control matrix with elution buffer, analysis of the recovery of tPA activity in the elution buffer shows that the Hb-AGE Sepharose is an efficient and selective affinity matrix for tPA (FIG. 5C).

In a next series of experiments the Hb-AGE Sepharose affinity matrix for proteins that bind to misfolded proteins, was used to capture the fraction in IgIV that binds specifically to misfolded proteins. IgIV that specifically bound to Hb-AGE-Sepharose was tested for binding to immobilized Hb-AGE and heat-denatured BSA, in an ELISA. First a standard curve of a dilution series of the IgIV stock was prepared using protein stain ADV01 (FIG. 5D). IgIV concentrations after contacting affinity matrix and after elution of bound protein from affinity matrix were determined using the IgIV standard curve. In a similar way, standard curves were prepared for the binding of dilution series of IgIV stock to immobilized Hb-AGE or heat-denatured BSA (FIG. 5E, H). Relative IgIV concentrations in IgIV after contacting affinity matrix or control matrix and in the eluates was determined by calculating IgIV concentrations using the standard curves. These calculated IgIV concentrations were compared with IgIV concentrations that were determined directly using ADV01 stain. With these numbers, an enrichment factor for specific binding of IgIV to misfolded proteins is calculated.

In FIG. 5F, binding of 1000 times diluted IgIV stock (50 μg/ml) and IgIV contacted with Hb-AGE-Sepharose or control-Sepharose to coated Hb-AGE is shown. Hb-AGE binding is approximately 50% reduced after contacting IgIV with Hb-AGE matrix, whereas no decrease in signal is observed after contacting IgIV with control matrix. Total protein concentrations after contacting Hb-AGE matrix or control matrix were 55 and 60 mg/ml. These deviations from the maximally expected value of 50 mg/ml (starting material) result from the non-linearity of the standard curve. In the IgIV fractions eluted from Hb-AGE-Sepharose and control matrix, 120 and 7 μg/ml IgIV was present, respectively, as determined with ADV01 protein stain. When binding of the 100 times diluted eluates to immobilized Hb-AGE was assessed, the observed signals corresponded to signals obtained after binding of approximately 75 and 0.3 mg/ml IgIV stock (FIG. 5G). Therefore, in conclusion, 120 μg/ml of Hb-AGE-Sepharose affinity matrix enriched IgIV binds Hb-AGE with a potency corresponding to approximately 75 mg/ml of the original IgIV stock. This corresponds to an enrichment factor of approximately 75.000/120=600 times. In FIG. 1 it is depicted that contacting IgIV with Hb-AGE-Sepharose or control-matrix does not reduce the signals obtained after assessing IgIV binding to immobilized heat-denatured BSA, when compared to starting material. However, when the 120 μg/ml IgIV that was eluted from the Hb-AGE matrix, was tested for binding to heat-denatured BSA, signals corresponded to signals obtained after binding of 1.7 mg/ml starting material (original IgIV stock) (FIG. 5J). This shows that contacting IgIV with misfolded protein affinity-matrix increases specificity for heat-denatured BSA with approximately 1700/120=14 times.

Alternative to Hb-AGE, other misfolded proteins are immobilized to a matrix in order to improve selectivity, affinity, capacity and/or stability of the affinity matrix. Alternative misfolded proteins that are immobilized are, but are not limited to, oxidized proteins, (heat-)denatured proteins, glycated proteins, proteins exposed to denaturing surfaces or denaturing molecules, e.g. CpG-ODN, lipopolysaccharides, dextran sulphate, kaolin, glass, lipids, or amyloid peptides, e.g. FP6, amyloid-β, FP13. Alternative to CNBr-Sepharose, other matrices or solid supports are applied for immobilization of the misfolded protein ligand. Preferably, the solid support is produced under good manufacturer practice (GMP) conditions, and preferably, the matrix is designated as a ‘Bioprocess medium’, referring to safety aspects of the matrix that are compatible with medical use with respect to humans. Other matrices/solid supports are, but are not limited to, NHS-Sepharose, Streptavidin-Sepharose, latex beads, epoxy activated solid support, e.g. cross-linked polymethacrylate, activated thiol Sepharose, Carboxylink, Profinity epoxide.

An affinity matrix is prepared using a misfolded protein that contributes to a specific disease. With this affinity matrix, those Ig's that bind the disease-associated misfolded protein with crossbeta structure conformation, are selectively isolated. After recovery of this Ig fraction, a disease-specific IgIV is obtained with higher specific beneficial outcome when used as therapy for the misfolding disease. Not only IgIV comprising solely IgG's is applied to this procedure, but every Ig fraction is tested for the presence of a beneficial subset of antibodies, e.g. antibodies of the IgM subclass. A few examples of misfolded proteins that are associated with a disease state and that are applied for the preparation of the IgIV enrichment affinity matrix are amyloid-β (Alzheimer's disease), glycated proteins (dialysis, diabetes), 62-microglobulin (dialysis), transthyretin (systemic amyloidosis). See for further examples of proteins that form misfolded crossbeta structure rich molecules and that are used for the disease-specific enrichment procedure, Tables 4 and 5.

New Constructs Combining High Specificity and Affinity for Misfolded Proteins with a Clearance Signal: Chimera of Misfolded Protein Binding Protein with Fc Domains of Ig's

Based on the findings that IgG molecules in IgIV and murine monoclonal IgG1/IgM/IgG2a antibodies bind to misfolded proteins with crossbeta structure conformation, a new molecule is designed with even higher specificity and/or affinity for misfolded proteins, combined with the ability to be prone to clearance via interaction with Fc receptors. For this purpose finger domains (F) or any other protein domain with affinity for crossbeta structure, e.g. an Ig domain of receptor for advanced glycation endproducts, a domain of (cluster II, cluster IV of) low density lipoprotein receptor related protein, a domain of the scavenger receptors A, -B-I or CD36, is fused at the DNA level or at the amino-acid level with an Fc portion of an Ig molecule. In fact, any of the proteins that has affinity for misfolded proteins provides a suitable domain to introduce specificity for crossbeta structure in the complex construct with the Fc domain (Table 4, 5). Finger domains of tPA, factor XII, hepatocyte growth factor activator and fibronectin all bind to misfolded proteins with crossbeta structure conformation, and are therefore all used for the design of chimeric constructs. Any combination of finger domains or stretches of multiple finger domains or combinations of finger domain(s) and other misfolded protein binding domains are also applied for the development of a chimeric construct with an Fc domain. The chimer gene is fused and prepared synthetically and is cloned in a suitable expression vector for expression purposes in for example yeast cells, plant cells, bacteria, eukaryotic cells, e.g. human embryonic kidney cells, baby hamster kidney cells. After purification of for example the recombinant F-Fc chimeric protein, it is applied as a therapeutic agent for any of the diseases for which IgIV has been used. Alternatively, affinity regions or synthetic molecules or any (portion of a) protein with affinity for crossbeta structure or for a protein comprising crossbeta structure are fused to for example Fc regions by any method known to a person skilled in the art for (non)-covalently coupling of protein (fragments). Moreover, non-proteinaceous molecules with affinity for crossbeta structure and/or molecules comprising crossbeta structure (Table 3) are fused to Fc regions in a similar way.

### Example 5

### Models to Test the Protective and/or Beneficial Effects of Administering IgIV, Affinity-Purified Enriched IgIV or Chimeric Structures of a Misfolded Protein Binding Protein or Molecule and an Fc Domain

To test a beneficial effect of IgIV, or an enriched IgIV fraction after affinity purification with a matrix with coupled misfolded protein, (humanized) anti-misfolded protein antibodies, or a chimeric structure of for example a finger domain and an Fc domain of an IgG molecule, several in vitro cell-based models for disease states, as well as in vivo animal or human models are applied to determine whether such modalities have a more pronounced beneficial effect than administering total IgIV or than current standard therapy.

In vitro murine dendritic cell assay (Auto)immunity is dependent on the presentation of (auto)antigens by antigen presenting cells, such as dendritic cells. Cultured murine dendritic cells (DC's) are thus applied as a model for (auto)immunogenicity. For this purpose, DC's are isolated from the hind legs of for example 8-12 weeks old Black-6 mice. Bones are isolated and rinsed in 70% ethanol, rinsed in RPMI-1640 medium with 25 mM HEPES, with 10% fetal calf serum, penicillin and Streptomycin. Then the bone is flushed with this buffer, in both directions. Eluates are cleared from erythrocytes by adding erythrocyte specific lysis buffer (obtained from the local UMC Utrecht Pharmacy Dept., catalogue number 97932329). Eluates are analyzed for viable cells by culturing them in cell culture plates. At this stage, the medium is enriched with 10 ng/ml GM-CSF. DC's growth in suspension or on a layer of macrophage cells. Using a FACS and specific antibodies, it is determined whether DC's are present and activated. Preferably the levels of so-called co-stimulatory molecules, such as B7.1, B7.2, MHC class II, CD40, CD80, CD86 are determined on preferably CD11c positive cells. Alternatively, activation of NF-κB and/or expression of cytokines is used as indicators of activation of cells involved in immunogenicity, such as APC and DC. Preferably, the following cytokines are quantified: TNFα, IL-1, IL-2, IL-6, and/or IFNγ. Preferably, the cytokine levels are quantified by ELISA. Alternatively, the mRNA levels are quantified. For a person skilled in the art it is evident that function of APC and DC are tested as well.

Alternatively, a stable DC line, cultured dendritic cells obtained from monocytes collected from human blood or other antigen presenting cells are used to test beneficial effects of depletion or neutralisation of misfolded proteins with crossbeta structure (Citterio et al., 1999).

Further experiments that are performed with DC's are exposure of the cells to lipopolysaccharide (LPS), followed by reading out levels of the above mentioned activation markers. The effect of pre- and/or co-incubations of LPS with (enriched) IgIV and/or other affinity regions before or during exposure of the LPS to DC's, is also tested. These experiments are seen as a model for bacterial infection and sepsis in humans.

### In Vitro Human Umbilical Vein Endothelial Cell Assay

Glycated proteins comprising crossbeta structure induce inflammatory response, believed to contribute to pathogenesis of certain diseases including diabetic nephropathy. In general, misfolded proteins induce cellular dysfunction with enhanced expression or activation of inflammatory signals. The effect of misfolded proteins on endothelial cell (dys)function is for example measured by determining the levels of reactive oxygen species in response to misfolded proteins. Human umbilical vein endothelial cells that are isolated and cultured, according to standard protocols, are used or other endothelial cells such as bEnd.3 endothelial cells. The levels of reactive oxygen species (ROS) levels are monitored using fluorescent probes, such as CM-H₂DCF-DA. Alternatively cell viability is monitored by MTT-assay. The cultured primary cells provide the opportunity to perform in vitro cell assays that are accepted in research community as model systems for certain disease states. Again, the ability of IgIV, isolated fractions thereof, a functional equivalent or our anti-crossbeta antibodies are applied in these systems.

### In Vivo Murine Model of Disseminated Intravascular Coagulation

Crossbeta structure induces disseminated intravascular coagulation (DIC). As a model for DIC, in female C57B1/6 mice the generalized Shwartzman's reaction is elicited. For this purpose, mice are injected with 5 μg lipopolysaccharide (LPS) in the footpad at day=0 and with 300 μg LPS intravenously at t=24 h. In time, survival is monitored, together with several plasma levels of proteins, e.g. cytokines.

### In Vivo Mouse/Rat Experimental Autoimmune Encephalomyelitis Model

To test whether anti-misfolded protein antibodies provide a beneficial effect during a multiple sclerosis (MS) relapse, an in vivo mouse model for MS, the experimental autoimmune (or allergic) encephalomyelitis (EAE) model is used. For this purpose, myelin basic protein (MBP) or myelin oligodendrocyte glycoprotein peptide 35-55 (MOG35-55) is emulsified in incomplete Freund's adjuvant (IFA) with mycobacterium. The presence of misfolded proteins is determined using Thioflavin T and Congo red fluorescence assays, as well as tPA binding and activation assays. Binding of IgIV or an enriched fraction of IgIV after affinity purification, to the emulsified MBP or MOG35-55 is assessed. To induce EAE in mice or rats, the emulsified MBP or MOG35-55 is injected in for example the hind footpad. In mice a subcutaneously injected amount of MOG35-55 is preferably accompanied with an intraperitoneal injection of Bordetella pertussis toxin, which is repeated after 48 h. For example, Lewis female rats are used, or female Balb/c mice. Measures for clinical disease are for example scored as follows: 0, normal; 1, limp tail; 2, impaired righting reflex; 3, paresis of hind limbs; 4, complete paralysis of hind limbs; 5, death. The effect of any (chimeric) antibody preparation is analyzed by administering the drug at one or more time points after inducing EAE. One of the preparations that is tested is IgIV that is affinity purified on an affinity matrix with immobilized denatured/misfolded MBP or MOG35-55, depending on which of the two proteins is used for inducing the disease.

### In Vivo Collagen Induced Arthritis Model

In the in vivo collagen induced arthritis model, rats are injected intradermally at the base of the tail and on the back above each leg with type II (bovine) collagen, dissolved in acetic acid and emulsified in IFA. The rats are daily examined for disease signs by monitoring swelling and erythema. One of the preparations that are tested is IgIV that is affinity purified on an affinity matrix with immobilized denatured/misfolded collagen in IFA.

### In Vivo Mouse Sepsis Model

Sepsis is mediated by crossbeta structure. One of the in vivo mouse sepsis models that is applied to test effects of IgIV, monoclonal antibodies or related drugs, is the ‘cecal ligation and puncture’ model. For this model, female Balb/c mice are anesthetized before an abdominal incision is made to bring the cecum outside the abdomen. After puncturing the cecum an amount of luminal contents is transferred outside through the punctures, before the cecum is returned in the adomen and the mouse is closed. Infection progression is monitored by measuring the body temperature and by scoring the mobility of mice. One considers mice lethally infected when they are hypothermic (T<33° C.) and when mice are unable to right themselves. Effects of administering antibodies with affinity for misfolded proteins with crossbeta structure conformation after the puncture of the cecum are assessed by monitoring a group of untreated mice and a group of mice that received an (enriched) IgIV, monoclonal antibodies or a chimeric construct.

### In Vivo Rat Sepsis Model

As an alternative for the mouse sepsis model, the rat sepsis model is used. For example, endotoxic shock is induced in Fischer rats of approximately 150 gr. by intravenous injection of 15 mg/kg Escherichia coli endotoxin. As a measure for disease progression, in ELISA's levels of tissue necrosis factor and interleukin-1 in blood are monitored. Effects of treatment with any preparation of IgIV or antibody or chimeric construct with affinity for unfolded protein are assessed this way.

### In Vivo Mouse Reactivated Streptococcus Cell Wall-Induced Arthritis Model

In an in vivo mouse model of reactivated Streptococcus cell wall-induced arthritis, C57BL/6 mice are induced by an intraarticular injection in the knee joints of cell walls of Streptococcus pyogenes T12 organisms. The injection is repeated five times with 1-week intervals. The disease progression is followed for example for about 40 days by measuring swelling of injected knee joints. After killing the mice, severity of the arthritis is scored macroscopically after removing the skin from the knee joints. Effects of administering anti-crossbeta structure antibodies or chimera are compared with controls that received no therapeutic and with control mice that were injected with buffer.

### In Vivo Mouse Experimental Rheumatoid Arthritis Model

In an in vivo mouse model for experimental collagen induced rheumatoid arthritis, for example male mice of the DBA/1 strain and/or male mice of the C57BL/6 strain are challenged with native bovine collagen type II. Arthritis is induced by injecting collagen emulsified in complete Freund's adjuvant with Mycobacterium tuberculosis, subcutaneously at the base of the tail. Mice are boosted at day 21 with collagen emulsified in IFA. Mice are monitored for evidence of arthritis and the severity of the disease is scored, using a standard scoring procedure. The effect of an antibody-based therapy is assessed by comparing control mice with arthritis and control mice that were injected twice with buffer only, with IgIV/monoclonal antibody/chimeric construct treated mice after induction of arthritis.

In Vivo Human Inflammation/Immunogenicity Model: Administration OF Glycated Protein +/−IgIV

Glycated proteins comprising crossbeta structure induce an inflammatory response, contributing to pathogenesis of certain diseases including diabetic nephropathy. In general, misfolded proteins induce cellular dysfunction with enhanced expression or activation of inflammatory signals. The inflammatory effects of misfolded proteins and anti-crossbeta structure reagents such as IgIV, fractions thereof, or functional equivalents inflammation are studied in mice and humans. Proteins comprising crossbeta structure are infused by intravenous administration. At different time intervals the effect on the level of acute phase proteins, such as C-reactive protein, Serum Amyloid A (SAA), Serum amyloid P-component (SAP) or complement factor 3 (C3) is measured. Alternatively the effect on other markers of inflammation, such as IL-6, IL-8, D-dimer or prothombin F1+2 levels is determined. Finally the levels of (auto)antibody formation are determined by ELISA.

### Whole Blood Assay for Determination of the Inflammatory or Immunogenic Nature of Compounds

One way of assessing whether activation of cells of the immune system by proteins with crossbeta structure conformation is blocked using crossbeta structure binding compounds, e.g. IgIV, monoclonal anti-crossbeta structure antibodies, chimeric constructs, is by use of a ‘whole blood’ assay. For this purpose, at day 1 freshly drawn human EDTA-blood is added in a 1:1 ratio to RPMI-1640 medium (HEPES buffered, with L-glutamine, Gibco, Invitrogen, Breda, The Netherlands), that is prewarmed at 37° C. Subsequently, proteins comprising crossbeta structure conformation, with or without crossbeta structure binding compounds, are added. Preferably, a positive control is included, preferably LPS. An inhibitor that is used for LPS is Polymyxin B, at 5 μg ml⁻¹ final concentration. Standard crossbeta structure conformation rich polypeptides that are tested are Aβ, amyloid γ-globulins, glycated proteins, FP13, heat-denatured OVA, heat-denatured BSA, heat-denatured MSA, heat-denatured lysozyme, and β2gpi exposed to cardiolipin. Negative controls are native γ-globulins, native albumin, native Hb, freshly dissolved Aβ or FP13, native OVA, other native proteins. As a control, all protein samples are tested in the absence or presence of 5 μg ml⁻¹ Polymyxin B to exclude effects seen due to putative endotoxin contaminations. In addition, native proteins alone or pre-exposed to denaturing adjuvants, e.g. LPS, and CPG-ODN, or other denaturing compounds or denaturing conditions (e.g. Cu²⁺-oxidation), are tested for immunogenic activity. All aforementioned tester compounds are tested in the absence and presence of a concentration series of a potential inhibitor of the inflammatory or immunogenic response, e.g. IgIV, monoclonal anti-crossbeta antibodies. The final volume of activators, controls and potential inhibitors added to the blood-medium mixture is approximately 1/200 of the total volume. Higher concentrations of activators and putative inhibitors are achieved by using concentrated RPMI-1640 medium for predilution steps (RPMI-1640 Medium powder, Gibco, Invitrogen; catalogue number 51800-035). The blood and the medium are mixed carefully and incubated overnight in a CO₂ incubator with lids that allow for the entrance of CO₂. At day 2 medium is collected after 10′ spinning at 1,000*g, at room temperature. The cell pellet is stored frozen. The medium is again spinned for 20′ at 2,000*g, at room temperature. Supernatant is analyzed using ELISAs for concentrations of markers of an immune response, e.g. tissue necrosis factor-α (TNFα), cytokines, chemokines. For example, TNFα levels after exposure of whole blood to tester compounds is assessed by using the commercially available TNF-alpha/TNFSF1A ELISA (R&D Systems, Minneapolis, Minn., USA; Human TNF-alpha Quantikine HS PharmPak). When positive and negative controls are established as well as a reliable titration curve, any solution is tested for the crossbeta structure load with respect to concentrations of markers for immunogenicity. Furthermore, putative inhibitors of the immune response are tested. For example, IgIV and monoclonal anti-crossbeta antibodies prevent an immune response upon addition to misfolded protein solutions.

### Phagocytosis of Cross-β Structure Comprising Moieties.

The uptake of cross-β structure comprising proteins, polypeptides and/or peptides as well as cells or cellular particles, and the effect of IgIV or a functional equivalent thereof are studied in vitro using cultured cells, preferably monocytes, dendritic cells, or macrophages or similar cells, for example U937 or THP-1 cells. Preferably, cross-β structure comprising molecules are labelled, preferably with 125I or a fluorescent label, preferably FITC, covalently attached to the molecule by a linker molecule, preferably ULS (universal Linkage system) or similar coupling method. Cells are preferably labelled with mepacrin or other fluorescent labels, such as rhodamine. Phagocytic cells are incubated in the presence of labelled cross-β structure comprising molecules or cells in the presence or absence of a cross-β structure binding compound, such as IgIV or functional equivalent thereof. After incubation, preferably during several hours, the uptake of labelled molecules or cells is measured preferably using a scintillation counter (for 125I) or by FACS-analysis (with fluorescent probes) or immunofluorescent microscopy. The uptake of cells is also counted under a light microscope with visual staining of the cells.

### Examples 6-20

### General Materials and Methods for Examples 6-20

Preparation of Misfolded Proteins with Crossbeta Structure

### Misfolding of Human IgIV

### IgIV Gammagard RF (IgIV RF)

IgIV Gammagard (native IgIV) was misfolded according to a procedure used to prepare antigen for rheumatoid factor (RF). IgIV Gammagard was dissolved under sterile conditions to 1 mg/ml in glycine buffer (100 mM glycine, 17 mM NaCl pH 8.2). It was heated for 5 minutes at 65° C. and stored at −80° C.

### Heat Denaturation of IgIV Gammagard (IgIV 65, IgIV 69, IgIV 76, Etc.)

IgIV Gammagard was dissolved under sterile conditions to 5 mg/ml in 20 mM sodium phosphate pH 5.0, and heat denatured from 25° C. to indicated temperatures with temperature steps of 5° C./minute. Final temperatures were 65° C., 69° C., 76° C., 80° C., 83° C. and 86° C. After heat denaturing, proteins were immediately stored at −80° C. and their structure was analyzed using various assays as described below. As native control, freshly dissolved IgIV Gammagard at a concentration of 5 mg/ml in 20 mM sodium phosphate pH 5.0 was kept at room temperature for 10 minutes, and stored at −80° C.

### HFIP/TFA Denaturation of IgIV Gammagard (IgIV HFIP/TFA)

IgIV Gammagard was dissolved under sterile conditions to 5 mg/ml in a 1:1 (v:v) mixture of 1,1,1,3,3,3-Hexafluoro-2-propanol (HFIP) and Trifluoroacetic acid (TFA). Subsequently, it was mixed thoroughly for 5 minutes on a vortex, at room temperature. The organic solvent was evaporated under N₂ gas and the dried material was dissolved to 1 mg/ml in H₂O and incubated for 7 days at 37° C., and stored at −20° C.

### Acid or Base Denaturation of IgIV Gammagard (IgIV Acid, IgIV Base)

IgIV Gammagard was dissolved to 5 mg/ml in PBS and incubated at room temperature on a roller device for 10 minutes. Then, the pH was lowered to pH 2 by addition of a volume of a 15% HCl stock in H₂O (acid denaturation) or elevated to pH 11 with a volume of a 5 M NaOH stock in H₂O (base denaturation), and incubated at 37° C. for 30 minutes. Then, the pH was adjusted to its initial, physiological value by adding 5 M NaOH or 15% HCl, respectively, and stored at −80° C.

### Misfolding of Octagam IgIV

Octagam IgIV (Octapharma, Brussel, Belgium, lot 5024018434, exp. December 2006) was used. The endotoxin concentration in IgIV was low, i.e. 0.13 E.U./ml in the 50 mg/ml Octagam stock, as determined using a standardized Limulus Amebocyte Lysate (LAL) assay (Cambrex). IgIV was diluted in 10 mM NaPi buffer (pH 8.1) to 1, 2.5, 5, 10 and 20 mg/ml and stepwise heated (0.5° C./minute) from 25° C. to 65° C., kept at room temperature for 1 hour and 40 minutes and subsequently stored at −80° C. Alternatively, IgIV was diluted in 10 mM HCl pH 2.0 and incubated at 65° C. for 6 hours. After this incubation, the pH was set to 7.3 with NaOH.

Acid or Base Denaturation of a Composition of Mouse IgGs (dmIgG Acid, dmIgG Base)

Mouse IgGs (mγ-globulins, from cohn fraction II, III approx. 99%, Sigma, lot 090k7680)

were dissolved to 1 mg/ml in PBS and incubated at room temperature on a roller device for 20 minutes. The IgGs were misfolded according to the method described above for IgIV ACID and IgIV BASE. The misfolded mγ-globulins is referred to as dmIgG or dmγ-globulins.
Misfolding of Mouse IgG by Heat (dmIgG 85° C.)

Mouse IgG composition was dissolved to 1 mg/ml in PBS and incubated at room temperature on a roller device for 20 minutes. Then, it was heated in steps of 5° C. per minute from 25° C. to 85° C. and subsequently stored at −80° C.

### Misfolding of a Composition of Human IgGs

Human IgGs (γ-globulins, Sigma, G4386) were dissolved to 5 mg/ml in HEPES buffer (20 mM

HEPES, 137 mM NaCl, 4 mM KCl, 3 mM CaCl₂). Then the pH was increased by adding a volume from a 5 M NaOH stock and kept for 40 minutes at 37° C. Then, an equal amount from a 5 M HCl stock was added to adjust pH to its initial value, and stored at −80° C. Large aggregates were observed by eye.

### Acid and Heat Denaturation of Apolipoprotein A-I

Apolipoprotein A-I (ApoA-I, 2.15 mg/ml, from human plasma, Sigma, A0722, lot 116K1408) in 10 mM NH₄HCO₃ and HCl added to 100 mM, was denatured by heating for 30 minutes at 37° C., 75° C. or 100° C. Subsequently, an equivalent amount of NaOH (100 mM final concentration) was added to change the pH to initial values.

### Base and Heat Denatured Apolipoprotein A-I

Again, 2.15 mg/ml Apolipoprotein A-I in 10 mM NH₄HCO₃, now with NaOH added to 100 mM, was denatured by heating for 30 minutes at 37° C., 75° C. or 100° C. Subsequently, an equivalent amount of HCl (100 mM final concentration) was added to change the pH to initial values.

### Heat Denatured Apolipoprotein A-I

The 2.15 mg/ml Apolipoprotein A-I (ApoA-I) stock in 10 mM NH₄HCO₃ was heat denatured for 30 minutes at 75° C. or 100° C.

Heat Denaturation of Ovalbumin (dOVA Std)

Ovalbumin (OVA, from chicken egg white, Sigma, A5503 grade V, lot 07147094) was dissolved in PBS at a concentration of 1 mg/ml, and heated from 30° C. to 85° C. for 5 cycles in a PCR machine with temperature steps of 5° C. per minute. This misfolded OVA is referred to as dOVA or dOVA standard (std).

Preparation of Fibrillar Amyloid Beta 1-42 (fAβ42)

Lyophilized synthetic human amyloid-β(1-42) peptide (DAEFRHDSGYEVHHQKLVFFAEDVGSNKGAIIGLMVGGVVIA; NKI Amsterdam, The Netherlands; SEQ-ID 9) (Aβ1-42) was first monomerized by dissolving at 1 mM in HFIP and aliquoted in sterile micro-centrifuge tubes. HFIP was removed with nitrogen gas, and the peptide film was resuspended in dry dimethyl sulfoxide (DMSO, Pierce, 20684) to a concentration of 5 mM, snap-frozen in liquid nitrogen and stored at −80° C. (monomerized Aβ1-42 stock). Thawed monomerized Aβ1-42 stock in DMSO was dissolved in 10 mM HCl at a final concentration of 400 μg/ml, and incubated for at 37° C. for 24 h, and subsequently stored at −80° C.

Aβ1-42 Dissolved in PBS and Directly Frozen at −80° C. at t=0 (Aβ42t=0)

Thawed monomerized Aβ1-42 stock in DMSO was dissolved in PBS, filter sterilized (0.22 μm), to a concentration of 100 μM, and stored at −80° C.

### Aβ1-42 Dissolved in HBS and Incubated for 24 h at 4° C. (Aβ42HBS)

Thawed monomerized Aβ1-42 stock in DMSO was dissolved in HBS (HEPES buffered saline, 137 mM NaCl, 4 mM KCl, 10 mM HEPES, pH 7.3) to a concentration of 100 μM. Buffer is filtrated by a 0.22 μm syringe filter prior use. Samples were stored at −80° C. after preparation.

Preparation of Fibrillar Amyloid Beta 1-40 (fAβ40)

Identical to Aβ1-42, a stock of monomerized synthetic human Aβ1-40 peptide (DAEFRHDSGYEVHHQKLVFFAEDVGSNKGAIIGLMVGGVV, NKI Amsterdam, The Netherlands) was prepared and stored at −80° C. Thawed monomerized Aβ1-40 in DMSO was dissolved in PBS to a concentration of 100 μM, and incubated for 168 h at room temperature, and subsequently stored at −80° C.

Aβ1-40 Dissolved in PBS and Directly Frozen at −80° C. at t=0 (Aβ40t=0)

Thawed monomerized Aβ 1-40 in DMSO was dissolved in PBS to a concentration of 100 μM, and directly stored at −80° C.

### Aβ1-40 Dissolved in 10 mM HCl and Incubated for 24 h at 37° C. (Aβ40HCl)

Thawed monomerized Aβ 1-40 in DMSO was dissolved in 10 mM HCl to a concentration of 100 μM, and incubated for 24 h at 37° C. Subsequently, it was neutralized with excess PBS1 (140 mM NaCl, 10 mM Na₂HPO₄, 1.8 mM KH₂PO₄, pH 7.4. PBS1 is filtrated using a 0.22 μm syringe filter prior to use) and stored at −80° C.

### Misfolding of Human Serum Albumin (Hsa, Cealb, Sanquin, The Netherlands, Lot 05C29H120A)

HSA, at 1, 2.5, 5, 10 and 20 mg/ml, pH 2 (lowered with a volume from a 5 M HCl stock) was heated at 65° C. for 6 h followed by neutralization with a volume from a 5 M NaOH stock, and subsequently stored at −80° C.

### Transmission Electron Microscopy (TEM)

TEM images were collected using a Jeol 1200 EX transmission electron microscope (Jeol Ltd., Tokyo, Japan) at an excitation voltage of 80 kV. For each sample, the formvar and carbon-coated side of a 100-mesh copper or nickel grid was positioned on a 5 μl drop of protein solution for 5 minutes. Afterwards, it was positioned on a 100 μl drop of PBS for 2 minutes, followed by three 2-minute incubations with a 100 μl drop of distilled water. The grids were then stained for 2 minutes with a 100 μl drop of 2% (m/v) methylcellulose with 0.4% uranyl acetate pH 4. Excess fluid was removed by streaking the side of the grids over filter paper, and the grids were subsequently dried under a lamp. Samples were analysed at a magnification of 10K.

### Congo Red (CR) Fluorescence Assay

Enhancement of Congo red fluorescence is a characteristic of misfolded proteins that comprise structural features common to proteins with crossbeta conformation. Fluorescence of Congo red (CR) (Aldrich Chemical Company, Inc., Milwaukee, Wis., USA, 86,095-6) was measured in duplo on a Thermo Fluoroskan Ascent 2.5 microplate fluorometer (Vantaa, Finland) in black 96-wells plates at an emission wavelength of 590 nm and an excitation wavelength of 544 nm. Protein and peptide stocks were diluted to 100 μg/ml for dOVA and IgIV samples and 40 μg/ml for Aβ samples in 25 μM CR in PBS, and incubated for 5 minutes at room temperature. Background fluorescence from buffer and protein solution without CR and from CR in buffer were subtracted form corresponding measurements of protein solution incubated with CR. Positive control for the measurements was 100 μg/ml dOVA (dOVA std).

### Thioflavin T (ThT) Fluorescence Enhancement Assay

Enhancement of ThT fluorescence is a characteristic of misfolded proteins that comprise structural features common to misfolded proteins with crossbeta conformation. Fluorescence of Thioflavin T (ThT) (Sigma, St. Louis, Mo., USA, T-3516) was measured similarly to the procedure described for CR. The emission wavelength was now 485 nm and the excitation wavelength was 435 nm. Protein and peptide stocks were diluted in 25 μM ThT in 50 mM Glycine buffer pH 9.0.

### 8-Anilino-1-Naphthalenesulfonic Acid (ANS) Fluorescence Assay

ANS fluorescence is enhanced when bound to clusters of hydrophobic amino-acyl residues. Upon binding to solvent-exposed hydrophobic regions of proteins, the emission wavelength (λ_{EM}) shifts from 514 nm to 460 nm when excited at a wavelength of 380 nm (λ_{EX}), accompanied by a dramatic enhancement in fluorescence intensity. Fluorescence of ANS (Sigma, A1028) was measured at an emission wavelength of 460 nm and an excitation wavelength of 380 nm. The various tester protein and peptide stock solutions were dissolved in 40 μM ANS in PBS and incubated for 5 minutes at room temperature. Background fluorescence from buffer and protein solution without ANS and of ANS in buffer were subtracted form corresponding measurements of protein solution incubated with ANS. Positive control for the measurements was 100 μg/ml dOVA (dOVA std).

4,4′ dianilio-1,1′ binaphthyl-5,5′ Disulfonic Acid Di-Potassium Salt (Bis-ANS) Fluorescence Enhancement Assay

Similar to CR, ThT and ANS, the enhancement of Bis-ANS (Sigma) fluorescence was measured. The emission wavelength was 485 nm and the excitation wavelength was 435 nm. Protein and peptide stocks were diluted in 25 μM Bis-ANS in PBS.

### Thioflavin S (ThS) Fluorescence Enhancement Assay

Enhancement of ThS fluorescence is a characteristic of misfolded proteins that comprise structural features common to proteins with crossbeta conformation. Fluorescence of ThS (Sigma, 033k1076) was measured according to the procedure described for CR and ThT. The emission wavelength was 542 nm and the excitation wavelength was 435 nm. Protein and peptide stocks were diluted in 25 μM ThS in PBS.

### Intrinsic Tryptophan Fluorescence Assay

Intrinsic tryptophan (Trp) fluorescence measurements were performed on a Gemini Spectramax XPS, (Molecular Devices) using Softmax pro v5.01 software, with 100 μl samples, in black 96-wells plates, at an excitation wavelength of 283 nm. Emission spectra were collected at room temperature in the 360-850 nm range. A natively folded protein either displays increased or decreased fluorescence compared to its misfolded counterpart. The absolute values of the TrP fluorescence intensity is not very informative. However, changes in the magnitude serve as a probing parameter for monitoring perturbations of the protein fold. A shift in the fluorescence emission wavelength is a better indication for local changes in the environment of the Trp fluorophore. Solvent exposed Trp residues display maximal fluorescence at 340-350 nm, whereas totally buried residues fluoresce at about 330 nm.

tPA/Plasminogen Activation Assay

Enhancement of tPA/plasminogen activity upon exposure of the two serine proteases to misfolded proteins was determined using a standardized chromogenic assay (see for example patent application WO2006101387, paragraph [0195], and Kranenburg et al., 2002, Curr. Biology 12(22), pp. 1833). Both tPA and plasminogen act in the Crossbeta Pathway (See Table 4). Enhancement of the activity of the crossbeta binding proteases is a measure for the presence of misfolded proteins comprising crossbeta structure.

Recombinant Fibronectin Finger Domains that Bind Misfolded Proteins

For a description of cloning, expression and purification of recombinant human fibronectin finger domains 4-5 (Fn F4-5), now with a C-terminal FLAG-tag and His-tag, see patent application WO2006101387 paragraph [0137]-[0165] and [0192-0194]). Protein expression in human embryonic kidney cells and purification was performed with the aid of the ABC-Expression Facility (University of Utrecht, The Netherlands). Purified Fn F4-5, at 288 μg/ml in PBS containing 5% glycerol, is stored at −80° C.

### tPA and Fibronectin Finger4-5 ELISA

For analysis of the binding of Fn F4-5 and tPA to the various human plasma ApoA-I preparations, standard ELISAs were applied as described above. For the analysis of tPA binding 10 mM ε-amino caproic acid was included in the binding buffer (PBS/0.1% Tween20). Binding of Fn F4-5-FLAG-His was determined using anti-FLAG antibody (mouse antibody, M2, peroxidase conjugate; Sigma, A-8592).

### Results

TEM Analysis of dOVA Standard

TEM analysis of heat-denatured ovalbumin, used as a standard misfolded protein in indicated assays (dOVA std.), shows that the misfolded protein aggregates into non-fibrillar multimers (not shown). For all fluorescence enhancement assays described above, as well as for the tPA/plasminogen activation assay, the dOVA std. concentration has been identified that results in maximum fluorescence enhancement, or maximum tPA/plasminogen activation, respectively. For the fluorescence enhancement assays, this concentration has been set to 100 μg/ml. For the tPA/plasminogen activation assay, 40 μg/ml dOVA std. is used as a reference. When appropriate, fluorescence enhancement and tPA/plasminogen activation induced by dOVA std. has been arbitrarily set to 100% for comparison purposes.

### TEM Analysis of Glycated BSA and Hb

FIG. 6 illustrates that misfolding of BSA and haemoglobin by glycation induces non-fibrillar amorphous aggregates.

### IgIV Octagram

FIG. 7 shows that denaturation of Octagam IgIV induces crossbeta structure. It is seen that various misfolding conditions result in misfolded proteins with varying TEM and Thioflavin T characteristics. Fibrils are not observed. It is concluded that at relatively high IgIV concentrations during misfolding, the size of the assemblies of IgIV molecules increases. This does not correlate with ThT fluorescence.

### IgIV Gammagard

Enhanced fluorescence of Thioflavin T, Congo red, ANS, Bis-ANS and Thioflavin S was observed with the various misfolded IgIV Gammagard samples in comparison with native IgIV (FIG. 8A-E). In general, an increase in fluorescence with the various fluorescent dyes is observed proportional to the increase in temperature during denaturation. Similar characteristics were observed when Trp fluorescence is measured (FIG. 8F). Elevated fluorescence is also observed for the base and acid denatured IgIV Gammagard, when compared to native IgIV Gammagard. It is seen that conditions for preparing epitopes for RF in IgG introduce a relatively small increase in crossbeta markers. For hIgG-BASE-37° C., ThT, CR and Trp fluorescence was measured. The increase in ThT fluorescence is moderate, but the increase in CR and Trp fluorescence is high, compared to native IgIV and compared to IgG misfolded upon alternative treatments.

TEM images at a magnification of 10K show that native IgIV Gammagard barely harbours any aggregates, and the aggregates present are amorphous and small in size (FIG. 9). When denaturing temperature increases, the aggregation size and abundance of the aggregates increase. Appearance of acid denatured IgIV Gammagard on a TEM image has most similarities with heat denatured IgIV Gammagard at a temperature of 76° C. Base denatured IgIV Gammagard show amorphous aggregates of an average size of 500 nm (FIG. 9J). Misfolded IgIV HFIP/TFA and base-denatured human γ-globulins appear as aggregates with similar features as seen for IgIV BASE (FIG. 9K, L). The number of aggregates is however higher and the average size of the multimeric assemblies is somewhat larger, compared to IgIV BASE. Especially with base-denatured γ-globulins (hIgG-BASE-37° C.), the average size of the multimers is about doubled when compared to IgIV BASE. IgIV RF appears as small dense and loose assemblies (FIG. 9B).

The potency of the misfolded preparations of IgIV Gammagard to activate tPA/plasminogen in a tPA mediated plasmin generation assay was examined (FIG. 9M). No tPA/plasminogen activation was observed with native IgIV Gammagard. Based on the tPA/plasminogen activation potency of the various denatured IgIV Gammagard preparations, three groups can be classified, namely moderate activators (IgIV RF, IgIV 65, IgIV 69 and IgIV Base), potent activators (IgIV 76, IgIV 80, IgIV 83 and IgIV 86) and very potent activators (IgIV Acid and IgIV HFIP/TFA). A striking difference in IgIV structure is noticed when the misfolding temperature is increased from 69° C. to 76° C. TEM images reveal that at 69° C. a few dense aggregates are formed (FIG. 9D) whereas at 76° C. relatively large and very dense assemblies are seen that increase in size when the misfolding temperature is further rising (FIG. 9E-H). This increase in size of the IgIV assemblies in accompanied by an increase in tPA/plasminogen activation, when misfolding at 69° C. and 76° C. are compared (FIG. 9M).

### Aβ Preparations

The various Aβ42 and Aβ40 preparations show enhanced ThT, CR and ANS fluorescence levels (FIG. 10). Aβ42HCl and Aβ40PBS1 appear as fibrillar aggregates on TEM images (FIG. 11C, F). Aβ40t=0, Aβ42t=0, Aβ40HCl and A642HBS appear as amorphous aggregates (FIG. 11A, B, D, E). Remarkably, the Aβ40PBS1 fibrils gave similar ThT fluorescence levels when compared to Aβ40HCl and Aβ40t=0, whereas the Aβ42HCl strongly increases ThT and CR fluorescence.

### Human Serum Albumin

As seen in FIG. 12A, denatured HSA at a concentration of 20 mg/ml enhances ThT fluorescence strongly, whereas at other concentrations no increase is seen in comparison with native HSA. No aggregates were observed by TEM analysis of native HSA or HSA denatured at 1 mg/ml (FIG. 12B, C). Amorphous aggregates, approximately 500 nm in size, were observed in denatured HSA at 2.5, 5 and 10 mg/ml (FIG. 12D-F). Aggregate size and the relative number of aggregates largely increases when HSA was denatured at 20 mg/ml (FIG. 12G).

### Mouse IgG

Enhanced fluorescence of ThT and CR was observed with the mouse IgG preparations that were misfolded using various methods, in comparison to native mouse IgG (FIG. 13). Thioflavin T and Congo red fluorescence are enhanced in the following order:

| | |
|---|---|
| ThT: | native IgG < IgG BASE < IgG ACID ≈ IgG 85° C. |
| Congo red: | native IgG << IgG BASE < IgG ACID < IgG 85° C. |

For the ThT signals differences between IgG BASE and IgG ACID compared to IgG 85° C. are more pronounced than for Congo red fluorescence signals. It is concluded that all three misfolding methods resulted in misfolding of the IgG accompanied by the formation of crossbeta structure.

Apopolipoprotein A-I

ApoA-I heat denatured at 100° C. in buffer with 100 mM NaOH, resulted in a slightly decrease in ThT fluorescence signal, as well as in CR fluorescence signal, when compared with native ApoA-I (FIGS. 14A and B). The observed decrease of ThT and CR fluorescence was not due to loss of protein as measured by A280 nm (FIG. 14C). FIG. 14B shows that CR fluorescence of 37° C. denatured ApoA-I in buffer with 100 mM NaOH (high pH) was slightly increased in comparison with native ApoA-I. Although no clear perceptible differences are observed in ThT or CR fluorescence intensities, significant differences are observed in the potency of the misfolded ApoA-I preparations to activate tPA/plasminogen in a tPA mediated plasminogen activation assay. The ApoA-I preparations that were heated to 37° C. or 75° C. are relatively moderate to potent activators of tPA/plasminogen (FIG. 14D). Misfolded ApoA-I at 100° C. is a very potent activator of tPA/plasminogen. In FIGS. 14E and F the results are displayed of the ELISA studies for determination of the presence of crossbeta structure and/or crossbeta induced conformation in the various preparations of human plasma ApoA-I. Native ApoA-I and ApoA-I with 100 mM NaOH added to the native ApoA-I stock, followed by heating to either 37° C., or 75° C. or 100° C., for 30 minutes, are incorporated in the studies. Half maximum binding of Fn F4-5 was reached with 110 μg/ml (native ApoA-I), 73 μg/ml (75° C.-misfolded ApoA-I), 48 μg/ml (100° C.-misfolded ApoA-I) and 5.2 μg/ml (HbAGE). For 37° C.-misfolded ApoA-I, no saturated binding was calculated. These figures and the curves show that upon misfolding of ApoA-I, the affinity for Fn F4-5 binding increases for ApoA-I misfolded at 75 or 100° C., accompanied with an increase in the total number of binding sites (Bmax). In addition, binding of tPA to the ApoA-I preparations is assessed. The highest number of binding sites for tPA (Bmax) is present on native ApoA-I, compared to the misfolded ApoA-I preparations. tPA does hardly bind to ApoA-I heated at high pH to 100° C. (no saturated binding detected). For native ApoA-I, 37° C.-misfolded ApoA-I, 75° C.-misfolded ApoA-I and HbAGE, half maximum binding is achieved with tPA concentrations of 4.3, 3.1, 1.6 and 3.5 nM, respectively, indicating that misfolding at 37° C. or 75° C. at basic conditions results in exposure of tPA binding sites with which tPA interacts with higher affinity, compared to native ApoA-I. The observation that tPA binds with relatively high affinity to native ApoA-I, with comparable measures as seen with HbAGE, shows that molecules with crossbeta structure and/or crossbeta induced conformation are already present in native ApoA-I. This finding is further substantiated by the observation that native ApoA-I displayes enhanced Congo red fluorescence and Thioflavin T fluorescence.

### Endotoxin Levels in Samples Used for Examples

Endotoxin levels in various solutions used for the experiments described in Examples 6 to 20 were determined with the Limulus Amebocyte Lysate (LAL) kit (Cambrex, QCL-1000). The kit was used according to the manufacturer's protocol, except that now measurements were performed using half of the described assay volume. As a reference lipopolysaccharide (LPS, Sigma, 2.5 mg/ml L-2630 clone 011:B4) was incorporated in several measurements. With the signals obtained with an LPS standard curve, an estimate of the endotoxin content in mass/volume was calculated with signals in endotoxin units (EU) obtained with unknown samples. In Table 6, endotoxin levels in EU are presented for the stock solutions.

### Example 6

### ‘Cross-Enrichment’: Enrichment of Human IgIV Towards Increased Affinity for Crossbeta Protein ‘A’ Also Results in Enriched IgIV with Increased Affinity for Crossbeta Protein ‘B’, ‘C’, ‘D’ . . .

We have shown before that Octagam IgIV enriched on BSA-AGE-matrix also has increased affinity for other misfolded proteins like Aβ40, Hb-AGE and dOVA (See Example 4). Now, we expanded this experiment by enriching IgIV on Aβ40/Aβ42 fibrils-matrix, BSA-AGE-matrix, dIgIV-matrix or dHSA-matrix and testing for binding of enriched IgIV to various misfolded crossbeta proteins. Misfolded proteins were immobilized to NHS-Sepharose. Enrichment factors with eluted IgIV from each of the affinity matrices were determined amongst others for binding to Aβ40/Aβ42 fibrils, Aβ aggregates, HSA, dHSA, BSA-AGE, dOVA, mγ-globulins and dmγ-globulins in an ELISA.

### Materials and Methods.

HSA (Cealb, Sanquin, The Netherlands, lot 05C29H120A) and IgIV (Octagam, Octapharma, lot 50244018432) at 1, 2.5, 5, 10 or 20 mg/ml were misfolded before immobilizing on NHS-Sepharose (GE-Healthcare). HSA was misfolded at pH 2 (HCl) by heating at 65° C. for 6 hours followed by neutralization with NaOH. IgIV was misfolded by stepwise heating (0.5° C. per min.) from 25° C. to 65° C., in 10 mM NaPi buffer (pH 8.1). NHS-Sepharose was washed 12 times with 1 mM HCl in Amicon filter cups (Millipore, UFC30SV00) before use. For immobilization purposes the five misfolded HSA preparations or IgIV preparations were mixed (1:2.5:5:10:20 mg/ml in a ratio of 5:4:3:2:1 (V:V:V:V:V)) and diluted 3× in immobilization buffer (0.5 M NaCl; 0.2 M NaHCO₃). BSA-AGE (10.25 mg/ml) and Aβ40/Aβ42 fibrils (0.28 mg/ml) were immobilized similarly. The fibrils were made as described in the Materials section. In brief Aβ40 was incubated for 186 h at 37° C., and Aβ42 was incubated for 24 h in HCl. These fibrils were mixed 1:1 in immobilization buffer. Matrix was incubated in immobilization buffer overnight and blocked with 0.1 M Tris pH 8.5. Matrix was washed 3× with 0.1 M Tris pH 8.5 and 3× with NaOAc 0.1 M; 0.5 M NaCl. These wash steps were repeated four times. The matrices were incubated with Octagam IgIV (50 mg/ml) for 4 h or overnight. IgIV flow-through (‘FT’) was collected and matrix was washed 12 times with HBS (HEPES-buffered saline, 140 mM NaCl, 10 mM HEPES, 45 mM CaCl₂, 0.005% Tween20, pH 7.4) before elution (2×1 hour in 1.140 M NaCl, 10 mM HEPES, 45 mM CaCl₂, 0.005% Tween20, pH 7.4; ‘eluate’). Eluates were dialyzed against HBS before further analysis.

The FT and eluate were tested for binding to various immobilized proteins using an ELISA: Aβ40/Aβ42 fibrils, Aβ40/Aβ42 non-fibrillar aggregates, HSA, dHSA, BSA-AGE, nOVA and dOVA. Four different Aβ40/Aβ42 non-fibrillar aggregates were prepared as described in the Materials section and mixed 1:1:1:1. at a concentration of 400 μg/ml. In short, Aβ40 was dissolved in PBS1 and frozen at −80° C. directly, Aβ40 was incubated for 24 h in HCl solution, Aβ42 was dissolved in PBS1 and frozen at −80° C. directly, and Aβ42 was dissolved in HBS and incubated for 24 h at 37° C. Enrichment factors were calculated as described in Example 4. Protein concentrations in the FT and eluates were determined using a BCA assay kit (Pierce, cat nr. 23223) and using Octagam IgIV for a standard curve.

### Results

FIG. 15 shows a typical result of an IgIV enrichment experiment using misfolded crossbeta protein-affinity matrices. Similar data was obtained for alternative combinations of enriched IgIV using matrix with misfolded protein X and immobilized protein Y, Z, . . . , as discussed below and as summarized in Table 7. In the illustrative example it is depicted that affinity regions that are selected using Aβ fibril-affinity matrix bind to various other misfolded proteins with different amino acid sequence and sequence length, e.g. BSA-AGE (FIG. 15). In addition, IgIV enriched on BSA-AGE-matrix has an enrichment factor of approximately 6 for binding to Aβ40/Aβ42 fibrils, compared to starting material (Octagam IgIV). In two similar experiments we obtained even higher enrichment factors (25 and 53) for binding of BSA-AGE matrix enriched IgIV to Aβ40/Aβ42 fibrils. IgIV enriched on Aβ40/Aβ42 fibril-matrix has an enrichment factor of 3 for binding to Aβ40/Aβ42 fibrils. With BSA-AGE matrix IgIV is more efficiently enriched for binding to Aβ40/Aβ42 than compared to the enrichment observed with an Aβ40/Aβ42 fibril matrix.

The enrichment factor for binding to BSA-AGE is on average highest for IgIV enriched on BSA-AGE-Sepharose. The enrichment factor for binding of IgIV enriched on Aβ40/Aβ42 fibrils matrix to BSA-AGE is approximately 5, as determined in three separate experiments (FIG. 15). The IgIV eluate of the BSA-AGE-Sepharose is also enriched for binding to dOVA (enrichment factor 3). In similar experiments also the IgIV eluate of the dIgIV-Sepharose and Aβ40/Aβ42 fibril-Sepharose were enriched for binding to dOVA with enrichment factors 1.5 and 6, respectively. This latter enrichment factor was not seen in one of the three consecutive studies. No enrichment was observed for binding to nOVA, indicating that with the enrichment procedure an IgIV sub-population is obtained that specifically binds to misfolded counterparts of proteins.

Enrichment factors for additional misfolded proteins were determined. A concentration series of Octagam IgIV starting material hardly binds to immobilized HSA, mγ-globulins and dmγ-globulins. Increased binding of Octagam IgIV to dHSA is seen when compared to binding to HSA. IgIV eluates of all misfolded protein matrices were enriched for binding to dHSA. Binding to Aβ40/Aβ42 misfolded non-fibrillar aggregates was most enhanced for IgIV enriched on Aβ40/Aβ42 fibrils matrix and BSA-AGE-Sepharose (enrichment factors of approximately 10). IgIV eluate of BSA-AGE-Sepharose is enriched for binding to misfolded mouse γ-globulins (dmγ-globulins).

Taken together we have shown that Octagam IgIV enriched on an affinity matrix comprising a misfolded crossbeta protein ‘A’ is enriched for binding to misfolded crossbeta protein ‘B’, ‘C’, etc., as well. With affinity matrices comprising Aβ40/Aβ42 fibrils or BSA-AGE, enriched IgIV with broadest spectrum specificity, expressed as relatively highest enrichment factors, for misfolded crossbeta proteins was obtained (Table 7). Most interestingly, for preparation of affinity matrices three non-fibrillar misfolded crossbeta proteins are incorporated in the studies, i.e. BSA-AGE, dHSA and dIgIV (see FIG. 6, 7, 12 in the General Materials and Methods section for TEM images).

Based on the results described here, a procedure is provided to select those affinity regions from a composition of affinity regions, that specifically bind to misfolded proteins comprising crossbeta structure, which specifically contribute to the pathology of a certain disease (See also FIG. 26). For this, in one embodiment a combination of two separate crossbeta-matrices with affinity for affinity regions that are capable of specifically binding misfolded proteins are consecutively applied. As described in more detail below, in either of two possible orders, a matrix I for selecting affinity regions that are capable of specifically interacting with any crossbeta structure and/or misfolded protein comprising a non-native 3-D structure and/or a crossbeta structure and/or amyloid, i.e. the Misfoldome, is used, as well as a matrix II with one or more selected misfolded proteins that contribute to the pathology of a disease of interest, for which therapeutic affinity regions are meant for treatment purposes, for selecting those affinity regions that are capable of specifically binding to the disease-related misfolded protein. When the matrices are applied in the order I→II, any set of proteins comprising a broad range of possible appearances of crossbeta structure or crossbeta structure induced conformations, and/or representative for the complete Misfoldome, either or not comprising those misfolded proteins that contribute to the pathology of the target protein misfolding disease, are used for preparation of the affinity matrix I. When the matrices are applied in the order II→I, the set of proteins comprising a broad range of possible appearances of crossbeta structure or crossbeta structure induced conformations, and/or proteins representative for the complete Misfoldome, do not comprise those misfolded proteins that contribute to the pathology of the target protein misfolding disease, that were implied for designing affinity matrix II. Of course, a skilled person is capable of designing alternative embodiments.

### Example 7

### Specific and Saturable Enrichment of IgIV for Affinity for Misfolded Crossbeta Proteins Using Affinity Matrices with Various Misfolded Crossbeta Proteins

In Example 4 HbAGE-matrix was used for isolation of a sub-population of immunoglobulins (Ig) with affinity for misfolded crossbeta proteins from Octagam IgIV. We tested the binding of enriched Octagam IgIV and the depleted residual, termed ‘Flow Through’ (FT), for binding to various crossbeta proteins. We observed that IgIV eluted from the affinity matrix is indeed enriched for binding to HbAGE (enrichment factor of 600) and that the FT is depleted for binding to HbAGE (enrichment factor of 0.5, or alternatively: depletion factor of 2.0). To test whether IgIV was specifically enriched on misfolded crossbeta protein-Sepharose for a sub-population of Ig molecules with specific affinity for the misfolded crossbeta protein (in the example above HbAGE) and not for matrix, in the current experiment the FT after incubation of IgIV with BSA-AGE-matrix was contacted again with a new portion of BSA-AGE-matrix, which was repeated in three successive steps. If binding of IgIV to BSA-AGE-matrix would be non-specific, the IgIV would be depleted without saturation in each consecutive step, ultimately ending with no Ig in the FT anymore. If binding is specific, less and less enriched IgIV will be obtained upon incubation of FT with a new amount of BSA-AGE-matrix.

### Materials and Methods.

Ten ml NHS-Sepharose matrix was washed 12× with 10 ml 1 mM HCl before coating of BSA-AGE. Coating was done overnight on a roller device at 4° C. by adding 2.5 ml immobilization solution (5.1 mg/ml BSA-AGE, 0.2 M NaHCO₃, 0.5 M NaCl, pH 8.3), followed by a block step with 0.1 M Tris pH 8.5 for 4 hours. To remove uncoated protein, several wash steps were performed, 3× with 0.1 M Tris pH 8.5 followed by 3× acidic buffer (0.1 M acetate, 0.5 M NaCl, pH 4.2). These wash steps were repeated 4 times. Beads were stored in HBS supplemented with 0.1% sodium azide. Before binding of Octagam (charge 5024018434), the matrix was washed 6× with HBS to remove the azide. To a portion of 2200 μl beads, 1100 μl Octagam IgIV (50 mg/ml) was added. The beads were incubated with Octagam for 1 hour and the FT fraction (FT1) was collected. Two hundred μl of this FT was saved and the remaining volume was applied to a fresh portion of BSA-AGE-matrix. The amount of affinity matrix was adjusted to the remaining volume of the FT, the amount of fresh matrix now being 1800 μl. Again the matrix was incubated for 1 hour with FT1 before centrifugation to collect the second FT fraction (FT2). This was repeated 4 times resulting in 4 FT fractions (FT1-FT4). All matrix samples incubated with the consecutive FT's were washed and bound Igs were eluted twice for 1 h, upon incubation with high salt (1.14 M NaCl, 10 mM HEPES, 4.5 mM CaCl₂, 0.005% Tween20, pH 7.4), resulting in 4 elution fractions (E1-E4). Also Aβ and dOVA were coupled to NHS-Sepharose matrix in the same way as described above. Aβ1-40 with Dutch type mutation E22Q was dissolved in PBS to a concentration of 1 mg/ml and incubated on a roller device at room temperature for 2 h, while protected from light with foil. The Aβ1-40 E22Q was incubated with matrix at a concentration of 0.66 mg/ml, in immobilization solution. For preparation dOVA-Sepharose affinity matrix, ovalbumin was denatured for 1 h at 100° C. in PBS at a concentration of 5 mg/ml, and immobilized on NHS-Sepharose in immobilization buffer at a concentration of 3.5 mg/ml. Thioflavin T and Congo red measurements confirmed formation of crossbeta structure in the misfolded dOVA sample upon heating at 100° C. Enrichment factors of the 4 FT fractions and the 4 eluates were determined in an ELISA as described before (Example 4). Immobilized misfolded crossbeta proteins were dOVA, Hb-AGE, BSA-AGE and Aβ40.

### Results and Discussion

Immobilization of extensively glucose-6-phosphate-glycated bovine serum albumin, misfolded crossbeta BSA-AGE, to NHS-Sepharose matrix resulted in an efficient affinity matrix for capturing BSA-AGE binding IgIV from Octagam (FIGS. 16A and B). It is shown that a fraction of Octagam IgIV binds specifically to the BSA-AGE-Sepharose. FT1 is depleted up to 85% for Ig molecules with affinity for BSA-AGE (enrichment factor 0.15). This number increases from 94.6% (enrichment factor 0.054) to 95% (enrichment factor 0.050) and 96.2 (enrichment factor 0.038) in the subsequent fractions FT2-4. The data show that efficient depletion of IgIV for molecules with affinity for BSA-AGE is achieved after a first contact of IgIV with BSA-AGE-Sepharose. To further test whether Ig molecules bound specifically to the BSA-AGE on the matrix, eluates (E1-4) were tested in an ELISA for binding to BSA-AGE and the enrichment factors were determined. E1 shows highest affinity for binding to BSA-AGE, as expected. The enrichment factor decreases from 41.3 for E1 to 13.7 for E2 and 11.8 for E3 to 8.7 for E4 in the subsequent binding steps in which subsequent FTs were contacted again with BSA-AGE matrix. This shows clearly that the amount of Ig molecules in the FT with affinity for BSA-AGE dramatically decreases already after the first contact with affinity matrix. This became even more clear in a similar experiment in which the FT fractions were applied 6 times to new affinity matrix. In this experiment the enrichment factor for FT became as low as 0.031, so basically no BSA-AGE binding properties remained (not shown). The absolute and relative amounts of IgIV in the eluates E1-4 were 89 μg (0.16%), 36 μg (0.23%), 18 μg (0.28%) and 17 μg (0.53%), respectively.

Also the binding properties of BSA-AGE-enriched IgIV and accompanying flow throughs to Aβ40, dOVA and HbAGE were determined. The FT fractions were neither depleted for binding to Aβ40 nor to dOVA, i.e. the enrichment factor stayed 1 (FIG. 16C, E). Without wishing to be bound to theory, a possible explanation for this observation is that upon enrichment with BSA-AGE matrix only those affinity regions are selected that comprise broad-range affinity for BSA-AGE, as well as for Aβ40 and for dOVA. Apparently, many Ig molecules with relatively high affinity for Aβ40 or dOVA, but less affinity for BSA-AGE remain in the FT fraction, explaining the modest depletion. FIGS. 16C and D, however, shows that still the eluates after contacting IgIV with BSA-AGE Sepharose are enriched for Ig molecules with affinity for Aβ40, despite being selected based on affinity for BSA-AGE. For dOVA an enrichment factor of 1.8 is observed with E1 (FIGS. 16E and F). The enrichment factor is lower for subsequent eluates, but not in parallel with the decreasing enrichment factors for binding to BSA-AGE in consecutive eluates. Apparently, the fraction of Ig molecules in Octagam IgIV with dual affinity for BSA-AGE as well as for dOVA is relatively small and thus enrichment with BSA-AGE matrix results only in little enrichment for binding to dOVA. Binding of the FT fractions and the eluate fractions to HbAGE follows similar patterns as seen with binding to BSA-AGE, showing overlapping epitopes on both misfolded crossbeta proteins (FIGS. 16G and 16H). In consecutive FT fractions the fraction of Ig molecules binding to HbAGE decreases dramatically. In parallel, the enrichment factor for binding of BSA-AGE-matrix enriched IgIV eluates to HbAGE decreases when comparing consecutive eluates.

In conclusion, these experiments show that with BSA-AGE affinity matrix Octagam IgIV is not only enriched for binding to BSA-AGE, but also for binding to other misfolded crossbeta proteins like Aβ40, HbAGE and dOVA. This shows that misfolded non-fibrillar crossbeta BSA-AGE comprises epitopes that are also exposed in the other three misfolded crossbeta proteins. It appears that IgIV comprises a fraction of affinity regions with affinity for BSA-AGE that does not have large overlap with a sub-population of Ig molecules with affinity for Aβ40 or for dOVA.

One advantage of using sub-optimal amounts of crossbeta protein-Sepharose is that in a first incubation only affinity regions with relatively high affinity will be selected. This is an advantage for purposes in which only high-affinity affinity regions should be used.

In a subsequent similar experiment, Aβ40-Sepharose and dOVA-Sepharose was used for six consecutive incubations of FTs (not shown). With the Aβ40 matrix, the FTs were depleted for binding to BSA-AGE, e.g. after 6 rounds of binding of successive FT fractions to fresh amounts of Aβ40-matrix, the ‘enrichment’ factor was 0.45. Binding of FTs after incubation with Aβ40-matrix to dOVA is less affected, the ‘enrichment’ factor is 0.83 after six binding steps. The eluates of the Aβ40-matrix are enriched for binding to BSA-AGE, with enrichment factors of 17, 4, 5, 3, 8, and 4. These eluates do not bind at all to dOVA. This shows that the sub-population of affinity regions in IgIV that bind to Aβ40 does overlap with the sub-population of Ig molecules that binds to BSA-AGE, but not with the sub-population of Ig molecules that binds to dOVA.

With dOVA-Sepharose, the depletion of Octagam IgIV FT for binding to dOVA is already sub-optimal (83%) with the applied ratio of affinity matrix and IgIV, i.e. with consecutive incubations of FTs with dOVA-matrix no further reduction in binding of the FTs to dOVA is achieved. The enrichment factors, which read in fact as ‘depletion’ factors, for binding of the FT fractions to BSA-AGE or Aβ40 are unaffected and stay around 0.8 for BSA-AGE and 1 for Aβ40. The eluates, however, are enriched for binding to BSA-AGE and Aβ40 (enrichment factors are 5 and 14, respectively). The enrichment factors do not decrease in eluates obtained during successive binding steps using the consecutive FTs.

Again, the experiments show that IgIV enriched using affinity matrix with misfolded crossbeta protein ‘A’ is also enriched for binding to misfolded crossbeta protein ‘B’. These experiments also show that with the used experimental settings the sub-population of Ig molecules in Octagam IgIV that binds to Aβ-Sepharose does not overlap with the sub-population of affinity regions in Octagam IgIV that binds dOVA. For BSA-AGE-Sepharose the absolute and relative amounts of enriched IgIV in eluates E1-6 were 31.5 μg (0.084%), 11.2 (0.062), 9.5 (0.098), 7.2 (0.16), 4.1 (0.145) and 0.27 μg (0.032%), respectively. For Aβ40 these numbers were 33.9 (0.09), 29.4 (0.17), 11.2 (0.11), 9.45 (0.21), 9.8 (0.35) and 3.8 μg (0.22%), respectively. For the dOVA matrix these figures were 27.6 (0.07), 22.4 (0.07), 21.8 (0.12), 17.1 (0.15), 11.5 (0.13) and 2.8 μg (0.06%).

The results also show that affinity regions with specificity for misfolded crossbeta proteins are specifically selected using an affinity matrix with immobilized misfolded crossbeta protein. Depletion of an amount of IgIV is saturable, that is to say, depletion of IgIV from a sub-population with specificity for misfolded proteins is achieved by using misfolded protein-matrix.

Although BSA, Hb, Aβ40 and OVA lack sequence homology and, in their native state, lack 3D structural homology, BSA-AGE, HbAGE, Aβ40 and dOVA share the presence of stretches of amino-acids with crossbeta conformation. Therefore, our results show that IgIV comprises a sub-population of affinity regions with broad spectrum affinity for crossbeta conformation or crossbeta induced conformation in various proteins that lack 3D structural homology in their native form and/or lack sequence homology. Moreover, the results with Aβ-Sepharose and subsequent dOVA ELISA's show that the crossbeta structure appears with varying structural details resulting in binding of different sub-populations of IgIV, but the same crossbeta dyes, i.e. Congo red, ThT (See ‘General Materials and Methods for Examples 6-20’.

### Example 8

### Binding of Octagam IgIV and Enriched IgIV, Obtained by Using an HbAGE-Affinity Matrix, to Fibrin, Aβ Aggregates and Misfolded Ovalbumin

### Materials & Methods

To test whether Octagam IgIV comprises Ig molecules with affinity for fibrin, which are polymers that comprise crossbeta structure, (see patent application US2007003552, paragraph [187, 188]), ELISAs are performed in which fibrin is formed in situ by incubating fibrinogen with thrombin/factor IIa in the wells of the ELISA plate. In addition, for comparison binding of IgIV to immobilized misfolded ovalbumin (dOVA) with characteristics of a protein with crossbeta structure (see Materials section) and to amyloid-β aggregates is assessed.

Ovalbumin (Sigma, A5503 grade V) was gently dissolved in PBS at a concentration of 1 mg/ml, incubated for 20 minutes at 37° C., subsequently 10 minutes at room temperature on a roller device, and stored at −80° C.→referred to as nOVA. nOVA was heated from 30° C. to 85° C. at 5° C. min⁻¹. This step was repeated four times, and denatured OVA was subsequently stored at −80° C.→referred to as dOVA std. For binding studies with dOVA std, 5 μg/ml dOVA std was coated. For analyzing the affinity of Octagam IgIV for immobilized Aβ, the Aβ40t=0 and Aβ42t=0 stocks were incorporated in the binding studies. Both Aβ preparations are coated at 5 μg/ml. Also HbAGE is coated at 5 μg/ml and analysis of binding to this crossbeta protein is determined as a positive control. For testing the binding of IgIV and tPA to immobilized fibrin with crossbeta conformation, the following protocol was applied to obtain wells of 96-wells ELISA plates with immobilized fibrin:
1. Prepare a 2 U/ml factor IIa stock in H₂O from a standard factor IIa/thrombin stock (human plasma, High Activity, Calbiochem, Germany, prod.nr 605195)
2. Prepare a 50 μg/ml fibrinogen solution (Fib3L 2170L in 20 mM sodium citrate-HCl pH 7.0, Kordia, The Netherlands) in PBS from a stock solution that is centrifuged for 10 minutes at 16.000*g before use.
3. Pipet 5 μl of factor IIa solution into the wells, add 100 μl of fibrinogen solution, or add 100 μl PBS to control wells. Final concentrations: [factor IIa]≈0.1 U/ml, [fibrinogen]≈47.5 μg/ml.
4. Incubate for 2 hours at room temperature with gentle aggitation. Coat controls are performed using anti-human fibrinogen antibody (DAKO-Cytomation, P0455).
5. Emptied wells are washed twice with TBS/0.1% Tween20. TBS: Tris-buffered saline with 150 mM NaCl, 50 mM Tris-HCl, pH 7.3.

First, wells coated with Aβ, dOVA, fibrin or control coat buffer are overlayed in triplicate with 50 μl/well of concentration series of IgIV or tPA for 1 hour at room temperature, with gentle agitation. In the tPA series, 10 mM εACA is included in the binding buffer to avoid binding of the kringle domains to exposed lysine and arginine residues of fibrin, and to direct the binding of the tPA finger domain to exposed crossbeta structure conformation. The signals obtained with fIIa coated control wells without fibrinogen that are overlayed with the concentration series tPA or IgIV, are subtracted from corresponding wells with immobilized fibrin. For all signals obtained with immobilized proteins with crossbeta structure, corresponding signals obtained with coat buffer coated wells are subtracted as background.

In a second series of experiments, binding of enriched IgIV that was obtained upon incubation of HbAGE-affinity matrix with Octagam IgIV, to fibrin was assessed.

### Results & Discussion/Conclusions

We previously determined that fibrin polymers exhibit features reminiscent to proteins with amyloid-like properties, such as binding of crossbeta specific dyes Congo red and Thioflavin T, and activation of tPA and plasminogen. We also determined that Octagam IgIV comprises a sub-population of Ig's that displays affinity for proteins with crossbeta structure. We therefore addressed whether IgIV binds fibrin in an ELISA. In FIG. 17 it is shown that indeed IgIV binds to positive control HbAGE, as was assessed earlier (see for example FIG. 1), as well as to dOVA, Aβ40 and Aβ42 preparations. Affinity for HbAGE is relatively high, whereas affinity for the latter three misfolded proteins is similar and somewhat lower. When fibrin is considered, both tPA and IgIV bind in a saturable manner. The half maximum binding of IgIV to fibrin is achieved at 200 μg/ml (approximately 1.3 μM) and this value is comparable to the values obtained with dOVA and Aβ preparations. These findings show that Octagam IgIV not only binds to the routinely used proteins comprising crossbeta structure, i.e. HbAGE, dOVA, Aβ, but also to the recently identified crossbeta-comprising molecules in fibrin.

These results show that Octagam IgIV comprises a sub-population of Ig's with affinity for fibrin. Hence, the use of this sub-population is beneficial in disorders in which prolonged lifetime of fibrin by competing of fibrin binding IgIV with tPA contributes to decreased disease symptoms or health problems, or in disorders in which hampered formation of fibrin is beneficial, which is achieved by introducing fibrin binding IgIV that interferes with polymerisation of fibrin monomers.

### Example 9

### Binding of IgIV Affinity Regions to Misfolded Human Plasma Apolipoprotein A-I

### Background

Amyloid in the menisci of the knee joint is one of the most common forms of localized amyloidosis and is especially increasingly prevalent in the elderly. The amyloid deposits can result in joint problems, that ultimately requires surgical action. Apolipoprotein A-I (ApoA-I) is detectable in the knee joints, forms amyloid and is implicated in a number of diseases and health problems, including joint problems. ApoA-I is the major protein component of high-density lipoprotein. Amyloid ApoA-I is also found in atherosclerotic plaques and arteries of atherosclerosis patients. Hence removal of misfolded ApoA-I from the circulation or elsewhere in the body is beneficial for patients suffering from diseases associated with amyloid ApoA-I. We tested whether affinity regions are able to bind misfolded ApoA-I and whether the disclosed means and methods are capable of selecting affinity regions enriched for those affinity regions binding to ApoA-I. The results displayed below show that indeed affinity regions recognize ApoA-I and that the disclosed methods and means are suitable for the isolation of affinity regions capable of binding ApoA-I. ApoA-I herewith serves as another example of a disease-associated protein for which affinity regions are isolated.

### Materials and Methods

For analysis of the binding properties of enriched IgIV that was obtained upon selection of affinity regions using HbAGE-Sepharose that was incubated with Octagam IgIV, towards human plasma ApoA-I, direct ELISAs are performed with immobilized ApoA-I preparations. For the studies, native ApoA-I is incorporated, and ApoA-I in 100 mM NaOH, heated for 30 minutes at either 37° C., or 75° C., 100° C., followed by pH adjustment with 5 M NaOH back to physiological pH. As can been seen in FIG. 14 the following order in relative positivity for selected crossbeta markers, i.e. enhancement of Congo red fluorescence, enhancement of ThT fluorescence, activation of tPA/plasminogen, binding of fibronectin finger4-5 and binding of tPA, is observed:

| | |
|---|---|
| Congo red: | 100° C. < native < 37° C. < 75° C. |
| ThT: | 100° C. < native < 75° C. < 37° C. |
| tPA/Plg act.: | background = native < 37° C. < 75° C. << 100° C. |
| Fn F4/5 binding: | native = 37° C. < 75° C. < 100° C. |
| tPA binding: | background = 100° C. < native ≈ 37° C. ≈ 75° C. |

From these comparisons it is clear that already native ApoA-I bears features of a misfolded protein with crossbeta structure, i.e. it enhances fluorescence of Congo red and ThT, and it binds tPA. In general, the ApoA-I preparations obtained by heating at 37° C. or 75° C. under basic conditions act as compositions with a relatively high content of crossbeta structure. However, when solely the potency to activate serine proteases (tPA/plasminogen) is considered, clearly ApoA-I heated to 100° C. is depicted as the composition with the highest ‘biologically active’ crossbeta content.

### Results and Discussion

In FIG. 18 binding curves for binding of enriched IgIV to native ApoA-I and three heat/base-misfolded preparations are displayed. When enriched IgIV is considered, kD's are in increasing order 1.3, 1.6, 2.0 and 2.8 μg/ml for ApoA-I 75° C., ApoA-I 37° C., native ApoA-I and ApoA-I 100° C., respectively. The number of binding sites is similar for native ApoA-I and ApoA-I 75° C., somewhat higher for ApoA-I 37° C., and much lower for ApoA-I 100° C. From A280 measurements it was concluded that the protein content in the four preparations is similar. Differences in maximum number of binding sites may be reflected by differences in coat efficiency. However, it is ApoA-I 100° C. that exposes most binding sites for Fn F4-5 (FIG. 14E). When affinity of enriched IgIV for the four ApoA-I preparations is compared to the affinity of Octagam IgIV, from which enriched IgIV has been selected, enrichment factors, calculated by dividing the kD's obtained with Octagam IgIV by the kD's obtained with enriched IgIV are 4.8 for both native ApoA-I and ApoA-I 75° C., whereas for ApoA-I-37° C. the enrichment factor is 12.8. For ApoA-I 100° C. an enrichment factor could not been determined, while no binding of Octagam IgIV has been detected, and modest binding of enriched IgIV. However, enrichment for binding to ApoA-I 100° C. is reflected by the binding characteristics as depicted in FIG. 18C.

In conclusion, the signals obtained with ‘native’ ApoA-I for crossbeta markers is reflected in binding characteristics of (enriched) IgIV, further substantiating the conclusion that the native ApoA-I comprises crossbeta structure, as it is purchased from the manufacturer. Furthermore, it is concluded that relative enhancement of both Congo red and ThT fluorescence upon contacting with ApoA-I preparations has predictive power with respect to expected binding characteristics of (enriched) IgIV, with ThT fluorescence showing the strongest correlation. From the ELISA data with enriched IgIV and ApoA-I heated to 37° C. it is concluded that this ApoA-I preparation comprises crossbeta structure or crossbeta structure induced protein conformation that has closest resemblance to the protein conformation of the HbAGE used for enrichment of IgIV, and/or the most comparable number of exposed crossbeta structure epitopes that serves as binding sites on ApoA-I for enriched IgIV. In general, the data show that by applying an appropriate crossbeta-affinity matrix, affinity regions are selected that bind to misfolded ApoA-I. In this way, a lead therapeutic affinity regions composition is obtained for use in treatment regimens of diseases or health problems related to the presence of misfolded ApoA-I, like for example treatment of pain caused by knee joint amyloidosis, dissolution of amyloid deposition in the arteries, and treatment of atherosclerosis accompanied by ApoA-I amyloid accumulation in plaques.

### Example 10

### Misfolded IgG Molecules Comprise the Target Epitope of Rheumatoid Factor, Auto-Antibodies Present in 70-80% of Rheumatoid Arthritis Patients

### Materials & Methods

### IgG Misfolding and Structure Analysis

We tested our hypothesis that human Rheumatoid Factor (RF), an auto-antibody present in 70-80% of Rheumatoid arthritis (RA) patients, binds to crossbeta or crossbeta-induced protein conformation in the IgG auto-antigen. We realized that it is common sense for detection of IgG binding by RF, which is mainly of IgM sub-class (although IgG and IgA RF also occurs), to presumably predominantly the Fc domains of its target auto-antigen, aggregation of the IgG upon heat-denaturation at 65° C. is a requirement. We warmed purified human IgG (Octagam IgIV) to 65° C. according to the procedures described in the General Materials and Methods section to Example 6-20, and analysed the structure by means of Congo red fluorescence, Thioflavin T fluorescence, ANS fluorescence and analysis of the binding and activation of tPA. Enhancement of Congo red and Thioflavin T fluorescence was determined with IgIV solutions diluted to 100 μg/ml. Subsequently, enhancement of tPA/plasminogen activity was determined using a standardized chromogenic assay (as described in patent application WO2006101387, paragraph [0195]). Binding of tPA in the presence of 10 mM ε-amino caproic acid to misfolded IgIV was assessed in a standard ELISA as described here before, with immobilized Aβ40t=0 as a positive control for tPA binding.

### Results

### Enhancement of Congo Red and ThT Fluorescence by Denatured IgG

The enhancement of Congo red fluorescence and Thioflavin T fluorescence was measured with heat-denatured misfolded IgIV. Based on the relative signals compared to control IgIV, heated IgIV is misfolded with accompanying hallmarks of a misfolded protein with crossbeta conformation (FIG. 19A, B).

### tPA Binding to Misfolded IgG

We observed that tPA, that is a component of the Crossbeta Pathway, binds to dIgIV (FIG. 19E). This observation further demonstrates that dIgIV is misfolded in a way that components of the Crossbeta Pathway recognize the newly introduced structural features.

tPA/Plg Activation by Misfolded IgG

Now that we observed binding of tPA to dIgIV we tested whether dIgIV activates tPA/plasminogen in a tPA/plasminogen chromogenic assay. Native IgIV does not induce tPA-mediated plasminogen activation (FIG. 19D). The heat-denatured misfolded IgIV samples however activate tPA/plasminogen, i.e. both the IgIV denatured at 65° C. in buffer with pH 2 (not shown), as well as the IgIV heat-denatured in NaPi buffer.

### Discussion

Introducing Misfolding with Crossbeta Structure in IgG Unmasks Epitopes in the Auto-Antigen of RF

Human IgG heated at 65° C. displays a series of structural characteristics commonly seen with amyloid-like misfolded proteins with crossbeta structure. The applied temperature is slightly above the temperature of 61° C. at which conformational changes are induced, according to differential scanning calorimetry measurements described previously by other investigators. Misfolded IgG enhances Congo red- and Thioflavin T fluorescence, binds tPA and activates tPA/plasminogen. To our knowledge, we are now the first to report that misfolded IgG auto-antigen for RF exposes neo-epitopes comprising structural properties reminiscent to amyloid with crossbeta conformation. In line with our observations, the reported fact that protease activity of tPA and factor XII, two serine proteases that bind to and are activated by proteins comprising crossbeta structure is increased in RA patients, is now explained.

Our observations point to RF as a useful source of human antibodies of the IgG, IgA and IgM classes that have specificity for crossbeta structure, and/or for crossbeta structure-induced conformations in proteins. Combined with our observations that a sub-population of Ig molecules in IgIV binds to misfolded IgIV molecules (See Table 7) and/or to misfolded mouse γ-globulins (see Example 19), we conclude that in fact the isolated sub-population in IgIV with affinity for misfolded Ig or misfolded proteins in general is reminiscent to RF. Both sources of affinity regions with affinity for misfolded IgG self-antigen are beneficial for development of affinity region-based therapeutics meant for treatment of diseases or health problems associated with the occurrence of misfolded IgG's.

### Example 11

### Determination of Relative Occurrence of Immunoglobulin Subclasses and IgG Isotypes in Various Preparations of Affinity Regions Methods

To determine the relative content of IgG isotypes IgG1, IgG2, IgG3 and IgG4 present in enriched IgIV obtained as eluate from an HbAGE affinity matrix, we concentrated 550 μl of the sample using Nanosep 10k centrifugal devices (Pall life science). The final concentration of concentrated enriched IgIV was 890 μg/ml, as determined by comparing absorbance at 280 nm with a standard curve determined with Octagam IgIV dilutions in PBS. Isotyping and determination of the relative abundance of Ig sub-classes was determined using standardized methods of the Laboratory for Medical Immunology (UMC Utrecht, The Netherlands), with the Image Immunochemistry nephelometer (Beckman Coulter). For comparison, Octagam IgIV from which enriched IgIV was extracted, was analyzed for relative abundance of IgG iso-types as well. In addition, appearance of Ig sub-classes was determined. Apart from the concentrated enriched IgIV sample, also non-concentrated material at 103 μg/ml in PBS was subjected to iso-typing and sub-class determinations. According to the manufacturer, Octagam IgIV, prepared from the Ig fraction of over 3500 human donors, consists of IgG's (≧95%), with a minor IgA fraction (≦0.4%) and a trace amount of ≦0.2% IgM. The distribution over the four IgG isotypes is: IgG1, 62.6%; IgG2, 30.1%; IgG3, 6.1%; IgG4, 1.2%. According to the manufacturer, in IgIV ≦3% of the Ig molecules is aggregated and over 90% of the molecules are monomers and dimers.

### Results & Discussion

For Octagam IgIV all seven measurements for subclass determination and isotyping of IgG are listed in Table 8. With Octagam IgIV it has been confirmed that indeed the majority of the Ig's is of the IgG sub-class. i.e. approximately 99.5%. The distribution over the four IgG iso-types is fairly close to what is reported in the Octagam IgIV datasheet (as described in Methods section). With non-concentrated enriched IgIV the subclass distribution could not be determined due to the lower detection limit of the Image Immunochemistry nephelometer. Determination of the relative presence of IgG2 was also hampered due to detection limits. Concentrations of IgG1, IgG3 and IgG4 could be determined (Table 8). The total Ig concentration in enriched IgIV was established to be 103 μg/ml, using the BCA protein concentration determination technique. With the nephelometer it was calculated that the total Ig concentration was 108 μg/ml. With concentrated enriched IgIV, concentrations for all four IgG iso-types could be determined, as well as total IgG content. IgA and IgM levels were lower than the detection limit. In the enriched IgIV fraction the relative abundance of IgG3, when compared to IgG1 as a reference, is approximately two-fold increased when compared to Octagam IgIV starting material from which enriched IgIV has been selected with HbAGE affinity matrix. The relative abundance of IgG2 and IgG4 when compared to the amount of IgG1 is hardly altered upon enrichment. So, in conclusion, a sub-population of IgG3 has relatively higher affinity for the HbAGE-Sepharose than the other iso-types.

Based on the result that all four IgG iso-types are determined in the enriched IgIV fraction, it is concluded that the Ig fraction consists of a mixture of at least four different human antibodies. The appearance of enriched IgIV as a smear on an iso-electric focusing gel under non-reducing conditions, also show that more than one monoclonal antibodies are present in the enriched IgIV selection (not shown). Concentration of IgA and IgM antibodies in the enriched affinity regions population could not be established, but the presence of trace amounts of one or more IgA and IgM clones can not be excluded based on the results.

### Example 12

### Analysis of the Influence of IgIV Affinity Regions on Platelet Aggregation induced by misfolded low density lipoprotein (oxLDL), and Analysis of the Binding of Enriched IgIV, Obtained from Octagam IgIV by Applying an HbAGE-Affinity Matrix, to oxLDL.

Modified LDL, for example due to oxidation (oxLDL) plays a prominent role in devastating diseases and health problems, like, for example atherosclerosis. We recently demonstrated that upon oxidation structural features are introduced in the protein portion of the LDL, i.e. ApoB-100, that are reminiscent to amyloid crossbeta conformation (see patent application WO2003NL00501). We now addressed the possibility that IgIV comprises affinity regions directed to the crossbeta conformation or crossbeta-induced conformation in human oxLDL, and even more preferably, in ApoB-100. For this study, enriched IgIV is used that has been obtained by extracting with HbAGE-Sepharose those affinity regions from Octagam human IgIV, that binds specifically to the immobilized misfolded protein (see Example 6, 7). In addition, Octagam IgIV is included in the studies.

### Materials and Methods

The influence of Octagam IgIV on activation of human blood platelets by oxLDL or by TRAP (thrombin receptor activating peptide, amino acids: SFLLRN) was assessed. The oxLDL has been prepared by incubating LDL purified from human blood with buffer comprising FeSO₄ (See Materials & Methods section of Example 2 for details). A degree of 56% oxidation was determined by measuring the diene content. As determined before (patent application WO2003NL00501), upon oxidation the oxLDL enhances Thioflavin T fluorescence (data not shown, see patent application US2007003552 for examples). Platelet aggregation was followed in time in an aggregometer (Chrono-Log Corporation, Havertown, Pa., USA) for 15 minutes at 37° C. at 900 rpm. A volume of 270 μl platelet suspension (200.000/μl) was incubated with 30 μl solution containing samples for analysis at indicated concentrations. For inhibition experiments with IgIV, 270 μl platelet suspension was incubated with 0.3 mg/ml Fibrinogen (plasminogen, fibronectin and von Willebrand factor depleted, Enzyme Research Laboratories, Lafayette, Ind., USA), 25 μl oxidized LDL, native LDL (nLDL) or TRAP solution and 5 μl solution with IgIV. In experiments with inhibitors, oxLDL, nLDL or TRAP were pre-incubated with increasing concentrations of IgIV, at 22° C. for 10 minutes. The maximal aggregation was expressed as a percentage of the response induced by 8 μM TRAP, that was arbitrarily set to 100%.

Binding of Octagam IgIV, depleted IgIV (flow-through after contacting IgIV with HbAGE-Sepharose) and enriched IgIV to oxLDL was assessed using an ELISA. As a positive control, binding of the affinity regions preparations was tested with immobilized BSA-AGE.

### Results & Discussion

In FIG. 20A it is seen that Octagam IgIV efficiently inhibits oxLDL-induced platelet activation and aggregation in a dose dependent manner. The IgIV does not influence aggregation of platelets upon activation with TRAP. The low level of aggregation seen upon exposing platelets to native LDL is not altered when the native LDL is pre-incubated with the concentration series of IgIV.

In a direct ELISA setting binding of IgIV affinity regions to oxidized LDL was assessed, and the relative affinity for oxLDL of IgIV that was enriched using misfolded HbAGE-affinity matrix was compared with the affinity of depleted IgIV, recovered as flow-through of the affinity matrix, and with Octagam IgIV starting material, used as a source for selecting affinity regions with affinity for misfolded crossbeta proteins. Binding characteristics are compared to those obtained with BSA-AGE, another misfolded protein. In FIG. 20 the results of the binding studies are depicted. Comparison of the binding properties of enriched IgIV and starting material towards BSA-AGE and oxLDL, shows that selection of affinity regions using HbAGE-matrix results in increased affinity of enriched IgIV for both misfolded proteins. The enrichment factor towards binding of glycated albumin or oxLDL, expressed as the ratio between the kD values obtained with binding of starting IgIV sample and the kD values obtained with enriched IgIV was calculated. For BSA-AGE, the enrichment factor is 45. For oxLDL, the enrichment factor is 27. The flow-through hardly binds to both misfolded proteins, indicating that again depletion of IgIV from affinity regions with specificity for misfolded proteins using HbAGE-Sepharose occurs rather efficiently, reminiscent to what has been described in Example 7

Antibodies, either passively administrated or induced by vaccination, are generally considered as good therapeutics for the treatment of an increasing number of diseases. Modified LDL, including oxidized LDL is a candidate target for the treatment of diseases, notably atherosclerosis, associated with increased formation and deposition of modified LDL. These results demonstrate that the disclosed method is capable of selecting affinity regions, such as human antibodies that preferentially bind modified proteins, comprising crossbeta structure characteristics, such as oxidized LDL. Such antibodies are preferably used for the detection and preferably treatment of diseases, such as atherosclerosis, associated with formation of misfolded proteins, preferably misfolded LDL as a consequence of modification, such as oxidation. In addition, those selected affinity regions are preferably used as model molecules displaying amino-acid sequences and 3D structural characteristics of affinity regions with affinity for misfolded proteins, for design of synthetic affinity regions (See Example 20).

In FIG. 20E, it is shown that the IgIV binds saturable to the oxLDL used for activation of the platelets. In FIG. 20G it is shown that affinity regions that are selected based on their affinity for misfolded Hb-AGE also bind with increased affinity to oxLDL, when compared to Octagam IgIV from which the enriched IgIV was selected. Together with the observed inhibitory effect of IgIV on oxLDL-induced platelet aggregation, our results show that IgIV comprises affinity regions with specificity for misfolded ApoB100 and that the affinity regions are able to interfere in responses of cells to misfolded proteins, i.e. in this Example 12 the aggregation of platelets upon exposure to oxLDL, a misfolded protein related to for example atherosclerosis.

### Example 13

### Role of Crossbeta Structure Binding Compounds Intravenous Immunoglobulins and Hepatocyte Growth Factor Activator Finger Domain on Bleeding Time in a Mouse Tail-Cut Experiment

### Materials & Methods

For the analysis of the influence of crossbeta structure binding compounds on in vivo coagulation and/or platelet aggregation, the mouse tail cut assay was performed to determine bleeding time. For this approach 50 11-13 weeks-old male black six C57BL/6JOlaHsd mice were used according to a protocol that was approved by the local ethical committee for animal experiments (Utrecht University, The Netherlands). Mice were injected intravenously (i.v.) in the tail vein with 100 μl buffer (PBS, control group, n=14) or buffer with tester compound or heparin (positive control, known to prolong bleeding). After 5-20 minutes the mice were anesthetized in a chamber with 5% Isofluran (induction), followed by anesthesia with 2-2.5% Isofluran using a mask during the course of the experiment (maintenance). Mice were kept at a warmed blanket (37° C.) with their tail hanging off the table. Five mm was cut from the tail with a scissors and blood was collected in cups. Time between injection and the tail cut was recorded, as well as the time between the start of bleeding and when bleeding (was) stopped. End points were arrest of bleeding, bleeding time lasting longer than 20 minutes, which was actively stopped by closing the wound by burning, and reaching a bled volume of over 200 μl due to fast bleeding. Prolonged bleeding for over 20 minutes and relatively excessive bleeding were both set arbitrarily to a bleeding time of 20 minutes. As a positive control for expected prolonged bleeding, we used 10 I.E./mouse heparin (Leo Pharmaceutical Products B.V., 5000 IE/ml) i.v. in 100 μl 0.9% NaCl (n=8). Hepatocyte growth factor activator (HGFA) finger/fibronectin type I domain was used at 4.7 mg/ml. Hundred μl was injected i.v. resulting in an approximate final concentration of 234 μg/ml based on an estimated blood volume of 2 ml/mouse (n=14). Human intravenous immunoglobulins (IgIV, Octagam, OctaPharma) from a 50 mg/ml stock as supplied by the manufacturer were used 20 times diluted (n=14).

For the studies a synthetic HGFA finger domain was used that was chemically synthesized according to standard procedures (Dr T. Hackeng, Academic Hospital Maastricht, The Netherlands; Hackeng, T. et al. (2001) Protein Sci. 10, 864-870, Hackeng, T. et al. (1997) Proc. Natl. Acad. Sci. U.S.A 94, 7845-7850). For HGFA, residues 200 to 240 (Swiss-Prot entry Q04756) were taken. The HGFA F domain can bind to misfolded proteins with crossbeta structure (see for example patent application WO2003NL00501).

### Results

Averaged time of bleeding from a tail wound after clipping off approximately 0.5 cm of the tail, of 14 mice of the buffer-treated, HGFA F treated and IgIV treated mice were determined (FIG. 21). Bleeding times were scored randomly by five different persons. Positive control for inducing prolonged bleeding time was heparin at a dose of 10 IE/mouse (n=8). In the reference group PBS was injected (n=14). Average bleeding time is 368 seconds for PBS-injected control mice and 1056 seconds for heparin-injected control mice. HGFA F and IgIV prolonged the bleeding time to on average 706 and 765 seconds. According to an unpaired t-test with two-tailed P values, bleeding times in HGFA F-injected mice and IgIV injected mice differ significantly from the bleeding time observed with PBS-injected mice (See FIG. 21). P values are 0.013 for HGFA F and 0.0045 for IgIV, respectively, when compared to the PBS-injected control group. These observations demonstrate a role for misfolded proteins with crossbeta structure in the cascades that result in coagulation and formation of a platelet plug. As depicted by us before (see for example patent application WO2003NL00501), fibrin polymerization requires crossbeta structure formation, and fibrin clot lysis by tPA and plasminogen is inhibited by crossbeta structure binding compounds. Furthermore, platelets are activated by misfolded proteins with crossbeta structure, and activated platelets themselves expose crossbeta structure. In Example 2 and 12 we show that IgIV interferes with crossbeta induced platelet aggregation. In Example 8 we demonstrate that IgIV enriched on HbAGE-Sepharose binds with increased specificity to fibrin, when compared to starting material used for IgIV enrichment. The data obtained now with HGFA F and IgIV in the tail clip bleeding assay show that these crossbeta binding molecules are a valuable starting point for the development of anti-coagulant therapeutics based on crossbeta structure binding compounds or based on compounds that bind to the molecules that bind to crossbeta structure during coagulation and platelet activation, and thereby facilitate coagulation and/or thrombus formation. In one embodiment of the suggested therapeutic, affinity regions with specificity for the proteins with crossbeta structure that contribute to coagulation and platelet aggregation are selected, thereby directing the therapeutic action more specifically to the proteins with crossbeta structure that underlie coagulation and/or thrombus formation.

### Example 14

### Isolation and Identification of Proteins from Plasma of Systemic Amyloidosis patients and serum and synovial fluid of RA patients, Using Matrix with Affinity Regions for Misfolded Proteins

Since crossbeta structures and proteins comprising a crossbeta structure are effectively bound to a collection of IgIV molecules according to the invention and/or to a composition according to the invention, they are effectively separated and/or isolated from a sample and/or an animal's or human's body and subsequently identified. IgIV after enrichment using crossbeta-affinity matrix, were used to isolate crossbeta structures and/or proteins comprising a crossbeta structure and/or proteins capable of specifically binding to crossbeta structure or crossbeta structure induced conformations in proteins. Proteins capable of specifically binding to a crossbeta structure and/or a crossbeta induced conformation in proteins are identified by the fact that when bound to protein with crossbeta structure and/or crossbeta induced conformation in an unsaturated manner, enriched IgIV matrices bind to the free binding sites on the protein with crossbeta and/or crossbeta induced conformation, thereby indirectly binding to the proteins binding to crossbeta structure or crossbeta structure induced conformation bound to the crossbeta structure and/or crossbeta induced conformation. The presence and/or identity of a crossbeta structure, and/or protein comprising a crossbeta structure and/or proteins capable of specifically binding to crossbeta structure or crossbeta structure induced conformations in proteins, of healthy individuals was compared with the presence and/or identity of a crossbeta structure, and/or protein comprising a crossbeta structure and/or proteins capable of specifically binding to crossbeta structure or crossbeta structure induced conformations in proteins, from individuals with a disease or health problem related to and/or associated with a crossbeta structure and/or a protein comprising a crossbeta structure and/or proteins capable of specifically binding to crossbeta structure or crossbeta structure induced conformations in proteins, like for example from individuals with primary AL amyloidosis or rheumatoid arthritis (RA). The identity of proteins isolated with a matrix of affinity regions was identified by mass-spectrometric analyses. The results of a sample originating from a healthy individual and a sample originating from a patient were compared. Furthermore, results obtained with a sample from a patient or a healthy individual contacted to enriched IgIV-matrix was compared to results obtained after contacting the same samples to control matrix without immobilised affinity regions. In this way, information was obtained about the identity and/or susceptibility of proteins prone to misfold and adopt crossbeta structure conformation during defined disease states, and about the protein(s) that preferentially bind(s) to those misfolded proteins. This provides key information for development of diagnostic tools that are disease specific, for instance to monitor disease state, to monitor effectiveness of therapy, to monitor occurrence of disease, and provides valuable leads for development of therapeutics targeted at crossbeta structures and/or protein(s) comprising a crossbeta structure and/or proteins capable of specifically binding to crossbeta structure or crossbeta structure induced conformations in proteins, which are preferably specific for the exemplary disorders. The therapeutics for instance clear the misfolded proteins in situ, or clear the misfolded proteins extracorporally, using for example affinity matrix during dialysis regimes.

### Material and Methods

Octagam IgIV (Octapharma, lot 5024018434) was enriched on Aβ-Sepharose, HbAGE-Sepharose and dIgIV-Sepharose, as described elsewhere in this application. The eluates of these matrices were dialysed against PBS (2 h, 1:2000, 4° C.), pooled and coated on CNBr-Sepharose (GE-Healthcare, Amersham Biosciences). Immobilization of enriched IgIV was performed essentially as described elsewhere in this application for NHS-Sepharose. CNBr-matrix was dissolved at 200 mg/ml in 1 mM HCl and treated the same as the NHS-matrix, except for an additional 5 minutes activation step in 1 mM HCl on a roller device before washing in this buffer. The pooled enriched fractions were diluted in immobilization buffer (50 mM NaCl and 40 mM NaHCO₃) to a concentration of 15 μg/ml. Control matrix was exposed to immobilization buffer, only. After overnight immobilization matrix was blocked with Tris and washed.

Six samples were incubated with the IgIV-Sepharose and the control-Sepharose: Normal pooled plasma, plasma of a patient I or of a patient II, with AL amyloidosis, serum of a patient III with RA (Rheumatoid Factor, RF titer 682), control serum and synovial fluid of a patient IV with RA (RF titer 23). All samples were diluted 20× in HBS and applied to 200 μl beads in two volumes of 500 μl. One volume was incubated for 4 h at RT and supernatant was discarded after centrifugation (2 minutes at 1400 rpm). Subsequently, the second volume was applied to the same matrix and incubated overnight on a roller device at 4° C. The affinity matrix or control matrix were washed 12 times with HBS and bound proteins were eluted with 2×50 μl of 8 M Urea in PBS, in two subsequent incubation steps of 1 h each. To collect the eluates, the matrices were centrifuged and the two eluates were pooled for each sample.

Sample codes:

A1 normal pooled plasma
C1 normal pooled plasma
A2 AL amyloidosis patient I
C2 AL amyloidosis patient I
A3 AL amyloidosis patient II
C3 AL amyloidosis patient II
A4 serum of patient III with RA (RF titer 682)
C4 serum of patient III with RA (RF titer 682)
A5 control serum
C5 control serum
A6 synovial fluid of patient IV with RA (RF titer 23)
C6 synovial fluid of patient IV with RA (RF titer 23)
A-series: affinity matrix of enriched IgIV-Sepharose
C-series: control matrix (activated/de-activated Sepharose)

Eluted proteins were reduced with dithiothreitol (DTT) (60 minutes, final concentration 6.5 mM) and then alkylated with iodoacetamide (30 minutes, final concentration 54 mM), followed by overnight tryptic digestion (10 ng/μl). Protein digests were desalted as described (Rappsilber et al 2003, Anal. Chem. 75, 663-670), vacuum dried and dissolved in 2.5% formic acid.

For analysis of peptide mixtures, an Agilent 1100 HPLC system (Agilent Technologies) connected to a Thermo Finnigan LTQ-MS (Thermo Electron, Bremen, Germany) was used. Protein digests were injected on a trap column (Reprosil C18 RP (Dr Maisch, Germany), 20 mm×100 μm I.D.) at 5 μl/minute. Subsequently, the peptides were transferred with a split-reduced flow rate of 100 nL/minute solvent A (0.1 M acetic acid) on the analytical column (Reprosil C18 RP, 20 cm×50 μm I.D.). Elution of the peptides was achieved with a linear gradient from 0 to 40% B (0.1 M acetic acid in 80% (v/v) acetonitrile) in 40 minutes. The column effluent was directly introduced into the ESI source of the mass spectrometer via a butt-connected nano-ESI emitter (New Objectives, Woburn, Mass.). The mass spectrometer was operated in the positive ion mode and parent ions were selected for fragmentation in data-dependent mode.

After mass spectrometric measurements, peak lists were generated using BioWorks software (Thermo Electron, Bremen, Germany). Protein identification was performed using Mascot software (www.matrixscience.com) by searching the IPIhuman database (version 3.24, downloaded from ftp://ftp.ebi.ac.uk/pub/databases/IPI/current) using the following settings: fully tryptic peptides, peptide tolerance 0.8 Da, MS/MS tolerance 0.9 Da, 1 missed cleavage allowed, carbamidomethyl (Cys) and oxidation (Met) as fixed and variable modification, respectively. The Scaffold software package (www.proteomesoftware.com) was used to parse the data and to filter peptides at a confidence level of 95%, allowing only protein identification with at least 2 peptides identified.

### Results & Discussion

In Table 9 the results are displayed for the different samples. For the amyloidosis patients human pooled plasma was used as a control. For the RA patient, serum from a healthy subject was used as a control. The results for control serum and normal pooled plasma are used for identification of peptides that are uniquely present in peptide compositions obtained with patient samples. The proteins displayed are the proteins or protein fragments which bound specifically from patient serum or plasma, compared to the control serum or plasma. Since there was no synovial fluid from a healthy subject available, only the control-matrix was used as a negative control for the synovial fluid from a RA patient. As mentioned, protein identification was performed by searching the IPIhuman database. IPI stands for ‘International Protein Index’, and is used to identify proteins, protein precursors and protein fragments in different databases, such as Swiss-Prot, TrEMBL, and PIR (these databases are all coupled in UniProt). IPI protein sets are made for a limited number of higher eukaryotic species whose genomic sequence has been completely determined but for which there are a large number of predicted protein sequences that are (not yet) listed in UniProt. IPI takes data from UniProt and also from sources comprising predictions, and combines them non-redundantly into a comprehensive proteome set for each species. This information was all accessed through the website of the European Bioinformatics Institute (EBI) which is accessible via: www.ebi.ac.uk.

One protein (IPI00807428) for which one peptide was identified in the eluate of control matrix that was contacted with synovial fluid is listed because seven peptides of this protein were identified in the eluate of enriched IgIV matrix. As seen, there are several ‘hypothetical’ proteins and proteins indicated by the molecular weight of the detected proteins. Because relatively short amino-acid sequences cannot always be attributed uniquely to a specific protein, which is especially seen among immunoglobulins, multiple results are possible for some of the protein fragments identified. In some other cases the IPI number of the hypothetical protein refers to an already identified protein.

In samples 2/3, the serum of AL amyloidosis patients I and II, one identified protein was ‘dynein heavy chain domain 3’. Dynein is a ‘motor protein’, which moves intracellular cargo's from the cell membrane into the cell. This is for instance the case with autophagy and axonal transport. Dynein is involved in transport of protein aggregates. So if it was for some reason bound to a protein aggregate in the plasma it could eventually end up binding the enriched IVIg-matrix. Therefore, dynein is identified as a crossbeta binding protein. In addition in sample 2/3, one hypothetical protein, two 25 kDa proteins and one immunoglobulin lambda constant 1 region were identified. The 25 kDa protein with IPI number IPI00747752 had no reference in any of the databases. It had however all the structural characteristics of immunoglobulins. The other 25 kDa protein had a gene reference to the immunoglobulin lambda locus. The hypothetical protein had a gene and a protein reference to immunoglobulin lambda variable 4-J. Immunoglobulins consist of two heavy chains, each with a constant region and an antigen binding variable region, and of two light chains also each with a constant region and an antigen binding variable region.

Because the patients suffer from primary AL amyloidosis the identified light chains are most likely the misfolded immunoglobulin light chains related to the pathology of the disease.

In sample 4, the serum from a RA patient III, several unique proteins were identified. This patient had an RF titer of over 600, indicating that this patient is suffering from severe RA. Four of the proteins identified were hypothetical proteins, one of which (IPI00760678) had a gene reference to the immunoglobulin lambda locus and a protein reference to the immunoglobulin lambda constant regions. The other three did have all the structural characteristics of immunoglobulins. Two proteins identified as 25 kDa proteins, both had a gene reference to the immunoglobulin lambda locus. One had a specific protein reference to the Rheumatoid Factor G9 light chain, a lambda variable 3 region apparently specific for Rheumatoid Factor. Therefore, it is concluded that this fragment is part of a crossbeta binding immunoglobulin. There were two proteins identified as immunoglobulin lambda constant 1 (IPI00658130, IPI00719373) and two proteins as immunoglobulin lambda constant 2 (IPI00555945, IPI00450309). There was one other protein identified as an immunoglobulin region, namely immunoglobulin lambda variable 3-25. It is concluded that this fragment comprises the amino-acid sequences which display affinity for misfolded proteins.

In different studies it was shown that Rheumatoid Factor in many cases contains specific lambda regions, one of which was apparently identified in this experiment. The other lambda regions identified also could be part of Rheumatoid Factor. These regions also could be part of misfolded immunoglobulin molecules, or they were part of the RF auto-antigen, which is the Fc region of immunoglobulins, that display characteristics of a misfolded protein comprising crossbeta structure (see Example 10).

Three other proteins were identified. One was identified as Isoform 1 of Centrosomal protein Cep290 (IPI00784201). Centrosome- and cilia-associated proteins play crucial roles in establishing polarity and regulating intracellular transport in post-mitotic cells. Due to its intracellular localisation, presence indicates that the content of lysed cells is present in the patient sample.

The second one was identified as the Isoform Gamma-β of the Fibrinogen gamma chain precursor (IP100021891). Different forms of fibrinogen are antigens for auto-antibodies in rheumatoid arthritis. The deiminated form of fibrinogen is one of these antigens, which is abundantly found in the synovial membrane of rheumatoid arthritis patients.

The final protein identified (IPI00004233) was the antigen to the monoclonal antibody Ki-67. This antigen is used as a proliferation marker. In some cases it is used as a marker for tumor growth. Most interestingly, it also has been described as a proliferation marker in rheumatoid arthritis, to assess the proliferation of inflammatory cell types in the synovium.

In sample 6, the synovial fluid of a rheumatoid arthritis patient IV, there were also several proteins identified uniquely. Three of these proteins were hypothetical proteins. One (IPI00807428) had no gene or protein references, but had all the characteristics of immunoglobulins. One (IPI00760678) had gene database references to the immunoglobulin lambda locus (constant 2) and protein database references to the immunoglobulin lambda locus constant region, but also protein references to the variable 2-14 region and to hypothetical proteins. The last one (IPI00003362) was in fact heat shock protein BiP (GRP78). BiP is one of the constituents of the Crossbeta Pathway and binds misfolded proteins (See Table 4 and 5). BiP is most likely identified in the patient sample because it was bound to a misfolded protein. BiP has also been identified as a target auto-antigen itself in RA patients.

Other than the hypothetical proteins, three other unnamed proteins were identified; two 25 kDa proteins and one 26 kDa protein. Both the 25 kDa proteins had gene references to the immunoglobulin lambda locus (IPI00747752, IPI00154742). One of these (IPI00154742) also had a protein reference to Rheumatoid factor G9 light chain, the lambda variable 3 region specific to rheumatoid factor, which was mentioned before. The 26 kDa protein had gene reference to immunoglobulin kappa variable 1-5.

There were also a few other immunoglobulin regions identified. One was an immunoglobulin kappa constant (IPI00807413), one (IPI00166866) an immunoglobulin heavy constant alpha 1, one (IPI00748998) an immunoglobulin single-chain Fv fragment (heavy chain variable region) and finally one (IPI00658130) which was identified as an immunoglobulin light chain constant 1.

The synovial fluid also contained some components of the complement system, namely Complement C1q subcomponent subunit C (IPI00022394), Complement C1r subcomponent (IPI00296165) and Complement factor H-related protein 1 (IPI00011264). It has been shown that in synovial fluid from rheumatoid arthritis patients, microparticles with bound C1q, C4 and/or C3 are abundantly found, compared to serum from both rheumatoid arthritis patients as well as healthy controls. It also has been found that C1q accumulates in amyloid beta plaques. Finally, C1q is structurally similar to surfactant protein A (SP-A), both having a globular head region and a collagen-like tail. SP-A has been associated with lamellar bodies in the synovium and autoantibodies to SP-A are present in the synovial fluid of rheumatoid arthritis patients. These auto-antibodies have some cross-reactivity with C1q. C1q is acting in the Crossbeta Pathway (See Table 4 and 5). Judging from the cross-reactivity of auto-antibodies against SP-A with C1q, it is also considered as being an auto-antigen. Especially because collagen is a common auto-antigen in rheumatoid arthritis.

Complement C1r is a serine protease which is capable of associating with C1q. C1r can activate other complement factors. No clear association with rheumatoid arthritis or protein misfolding has been found thus far.

Complement factor H-related protein 1 (FHR-1) consists of five short consensus repeats (also found in factor H) and its function is unknown thus far. FHR-1 is found in human plasma as part of certain lipoprotein particles. It was shown that FHR-1 is associated with a lipoprotein complex of phospholipid and other proteins in plasma and that this complex mediates responses of cells to lypopolysaccharides (LPS). We demonstrated that LPS induces crossbeta conformation in proteins. We also established that ApoA-I is capable of adopting crossbeta conformation. In addition, ApoA-I is capable of binding to other proteins comprising crossbeta conformation. The lipoprotein in the complex consists of phospholipids, apolipoprotein A-I (apoAI), lipopolysaccharide binding protein (LBP), and factor H-related proteins (FHRs). It is concluded that FHR-1 plays a role in carrying and/or regulating the function of LBP. As FHR-1 is the dominant protein component of these particles, FHR-1 appears several fold more abundant than either ApoA-I or LBP. Previously, it was shown that a related protein composed of six short consensus repeats known as beta 2-glycoprotein I (also called apolipoprotein H) associates both with HDL particles and with phospholipids.

Beta 2-glycoprotein I (IPI00298828) was also identified in the synovial fluid sample. Beta 2-glycoprotein I is a known auto-antigen in atherosclerosis and anti-phospholipid syndrome, a condition with increased risk for thrombosis. The functions of beta 2-glycoprotein I remain unclear. It however has been shown that it inhibits phospholipid-dependent coagulation reactions, such as the activity of the pro-thrombinase—tenase complex, and factor XII activation. It also binds factor XI and inhibits its activation. In contrast, it inhibits anti-coagulant activity of activated protein C and it may contribute to thrombin generation in vivo. When beta 2-glycoprotein I is cleaved by plasmin, it binds plasminogen and suppresses plasmin generation. We showed that β2gpi comprises crossbeta conformation when contacted with cardiolipin or when alkylated, rendering it with immunogenic potential.

There were three other proteins identified in the synoval fluid sample, namely calmodulin-like protein 5 (IPI00021536) (also called calmodulin-like skin protein), isoform I of desmoplakin (DPI) (IPI00013933) and isoform I of gelsolin (IPI00026314). Calmodulin-like protein 5 is a skin specific calcium binding protein and its expression is restricted to the stratum granulosum and the lower layers of the stratum corneum. It is expressed during cell differentiation. This protein is probably present in the synovial fluid as a contamination (skin cells). Desmoplakin is a regulator of microtubule organisation in the epidermis and it associates with keratins of the epidermis. This protein is probably also a contamination. Gelsolin caps actin filaments, and a secreted form of gelsolin is present in plasma where it probably acts as an actin-scavenger. Gelsolin is also capable of forming amyloid deposits and is one of the proteins causing cerebral amyloid angiopathy. Mutations in the gelsolin gene result in the Finnish type of gelsolin-related familial amyloidosis. When gelsolin aggregates or misfolded gelsolin was present in the synovial fluid sample, it is not surprising that it bound to the enriched IVIg-matrix.

By the use of the enriched IgIV-affinity matrix, as described in the current Example, we identified several proteins unique for amyloidosis patients, and series of proteins was uniquely identified in samples obtained from patients with rheumatoid arthritis. These proteins either contain a crossbeta structure or are crossbeta binding proteins, themselves. These proteins form the basis for the development of a disease-specific diagnostic tool and/or are newly identified targets for the development of therapeutics aimed at depleting patients from disease-modulating misfolded proteins in vivo (for instance by administering drugs) and/or ex vivo (e.g. extracorporal device). Moreover, the studies revealed insight into several identified crossbeta binding molecules apparently related to the disease. The identified variable regions of Ig's serve as a good starting point for development of synthetic affinity regions (see below, Example 20).

### Example 15

### Modulation of the Interaction of Misfolded Proteins with Cells by Affinity Regions

Misfolded proteins comprising crossbeta structure are capable of binding to cells and evoke cellular responses, including but not limited to inflammatory responses and changes in cell growth or apoptosis. We addressed whether affinity regions modulate the interaction of such misfolded proteins with cells. We used human primary endothelial cells (HUVECs) isolated from umbilical veins.

### Materials & Methods

### Isolation, Culturing and Analysis of Human Umbilical Vein Endothelial Cells (HUVECs)

### Isolation and Culturing

HUVECs are primary endothelial cells (ECs), isolated from umbilical cords using 0.1% collagenase (Sigma, C0130, 100 mg, dissolved in 100 ml M199 medium supplemented with 10% FCS (Gibco 10106-169) and Penicillin-Streptomycin (P/S, Gibco, 15140-122)), according to widespread used standard procedures known to a person skilled in the art. HUVECs have the typical features of ECs, e.g. cobblestone morphology and von Willebrand factor storage in Weibel-Palade bodies. HUVECs can regularly be cultured up to passage 5; beyond passage 5 HUVECs loose typical EC markers. The isolation is described here in brief. The umbilical cord is washed for less than 3 minutes in ethanol and subsequently with PBS. The vein is connected to canules and flushed with 10 ml PBS, followed by loading with the 0.1% collagenase solution. After a 15 minute-incubation at 37° C., the detached endothelial cell suspension is recovered by flushing the vein with 10 ml medium which is subsequently added to the collagenase solution. The EC suspension is centrifuged for 5 minutes at room temperature, at low g-force. Supernatant is discarded and the cell pellet is resuspended in 5 ml ‘rich medium’ (EGM-2; Endothelial basal medium (EBM-2, Cambrex, CC-3156) and Singlequots containing supplements for endothelial cells (Cambrex, CC-4176)). Cells (passage 0, P0) are seeded in a culture flask coated with 0.5% gelatin (Sigma, G1393). To facilitate the adhesion of the endothelial cells, human fibronectin is added to the cell culture at a final concentration of 2 μg/ml. EC's are cultured at 37° C., at 5% CO₂. The cell culture medium is refreshed every 2-3 days up to confluency. Then, with the addition of trypsin-EDTA, the cells are detached from the flask, centrifuged at low g-force, resuspended in rich medium and seeded in larger 0.5% gelatin-precoated cell culture flasks.

### Expression and Purification of Rage

For a description of recombinant human sRAGE cloning, expression and purification, see patent application WO2006101387 (paragraph [0303]). Purified sRAGE-FLAG-His stock was 284 μg/ml in PBS, stored at −80° C.

### Adhesion of Cells to Misfolded Proteins

In 96-wells plates (Immulon 1B Thermo Labsystems 3355) proteins, i.e. BSA-AGE (5 μg/ml), 10 μg/ml native IVIg (Octagam charge#5024018434), 10 μg/ml enriched IVIg (enriched by contacting Octagam IgIV with Hb-AGE-Sepharose [see elsewhere in the application for description]) or gelatin (Sigma G1393, 2% solution in H₂O or PBS, positive control for adhesion to ECs) were coated using 100 μl solutions. Following incubation for 2 hours at 37° C. the solutions were discarded and the wells blocked for 1 hour at 37° C. with 100 μl/well of 0.5% polyvinylpyrrolidone (PVP, Sigma P5288) in PBS, filter (0.22 μm) sterilized. PVP is an inert polymer that does not support cell adhesion. Subsequently, the solution with PVP was discarded. Next, the plates were incubated with 40 μl RPMI 1640 medium (Gibco 52400) and 10 μl of potential inhibitor, such as affinity regions. HUVECs were obtained by trypsinization. After centrifugation cells were resuspended in RPMI 1640 medium with P/S and diluted to 80.000-100.000 cells/ml. Each well was seeded with 100 μl of the cell suspension. Cells were allowed to adhere for 1 hour at 37° C. Plates were gently washed with RPMI 1640 medium with P/S. The medium was removed by pipetting along the wall of the wells. Plates were washed until blank wells contained hardly any cells, i.e. 1-3 times. Subsequently, 50 μl RPMI medium was added to each well, followed by the addition of 5 μl/well 10% Triton-X100 in PBS and incubation for 10 min at 37° C. Next, 50 ul of lactodehydrogenase (LDH, Roche Applied Science, 11644793001) solution was added according to instructions of the manufacturer. The plate was incubated for 0.5-3 hours at room temperature in the dark. The absorbance at 490 nm was measured on a Versamax microplate reader at various time points.

### Binding of Misfolded Proteins to Cells Assessed by Fluorescence-Activated Cell-Sorting (FACS) Analysis

For these experiments HUVECs were isolated by trypsinization. After trypsinization cells were collected in RPMI 1640, containing P/S and 10% FCS and centrifuged. After centrifugation cells were resuspended in RPMI medium without FCS at a concentration of 250.000 cells/250 μl. Individual 4-ml tubes (polypropylene, Greiner), containing 250 μl cell suspensions were made. To each tube 75 μl of a sample, containing either buffer (PBS) only, 50 μl buffer with 25 μl oxLDL (1 mg/ml) or 74 μl buffer with 1 μl BSA-AGE (25 mg/ml), was added. Subsequently, the cells were incubated with the sample for approximately 3.5 hours at 4° C. Next, cells were pelleted by centrifugation and the supernatant was discarded. Cells were washed subsequently with FACS buffer (PBS/0.5% BSA/0.05% m/v NaN₃) at 4° C. and resuspended in FACS-buffer at approximately 1×10⁵ cells/100 μl for subsequent analysis. Cell death was determined by adding 3 μl 7-aminoactinomycin D (7AAD) solution (prepared according to standard procedures). Binding of sample BSA-AGE (see elsewhere in this application for preparation details) was determined with anti-AGE monoclonal antibody 4B5 (10 μg/ml) and, after washing, with goat anti-mouse PE secondary antibodies (Jackson Immunoresearch, West Grove, USA). Binding of BSA-AGE was also assessed using the intrinsic fluorescence of BSA-AGE in the PE channel. Binding of oxidized LDL (oxLDL, oxidized for 56% following incubation with FeSO₄; specific enhancement of Thioflavin T fluorescence) was determined with rabbit serum with anti-ApoB100 polyclonal antibodies (Dade Behring, Newark, Del., USA, lot. 153670) at a concentration of 160 μg/ml and, after washing the cells, with FITC-labelled goat anti-rabbit:antibodies (1:200, Jackson).

### Results & Conclusion

### Adhesion of Cells to Misfolded Proteins

It was found that HUVECs adhere to misfolded proteins, i.e. as shown here with BSA-AGE, to a somewhat greater extent, approximately 125%, than to gelatin (FIG. 22A, bars 1 vs. 3). Increasing concentrations of affinity regions, i.e. IgIV, inhibited adhesion of ECs to BSA-AGE (bars 7-9 vs. bar 3). These data reveal that affinity regions interfere with the interaction of misfolded proteins with cells.

FIG. 22B shows that cells also bind to affinity regions (IVIg, Octagam), most efficiently to enriched affinity regions (enriched IVIg, after enrichment by contacting Octagam with Hb-AGE-Sepharose, see elsewhere in this application for description). Binding of ECs to the immobilized affinity regions comprising Fc domains is not mediated by classical Fc receptors, since such receptors, i.e. CD16, CD32a and b and CD64, were not present on the cells, as determined using FACS analysis (not shown). Since affinity regions are capable of specifically binding misfolded proteins, this interaction between affinity regions and cells is explained by binding of misfolded proteins on the cells to the affinity regions, specifically. Indeed, approximately 1-2% of the cells was less viable, as determined with FACS (not shown).

### Binding of Misfolded Proteins to Cells Determined by Flow Cytometry

Using two methods, BSA-AGE was found to bind efficiently to 96% of the ECs with a mean fluorescence intensity (MFI) of 13.9. OxLDL bound to 18% of the incubated ECs and displayed an MFI of 1.6. The binding characteristics obtained with ECs incubated in suspension with BSA-AGE are in line with the observation that ECs bind efficiently to wells of cell culture plates that are coated with BSA-AGE (see FIG. 22).

Taken together, these results demonstrate that cells are capable of specifically binding misfolded proteins with crossbeta structure and that affinity regions, preferably enriched affinity regions, modulate the interaction of such misfolded proteins with cells. In this Example, we observed that IgIV affinity regions present in Octagam IgIV efficiently block the adhesion of ECs to immobilized misfolded BSA-AGE. It is concluded that affinity regions directed against the immobilized misfolded protein bind and shield the misfolded protein from interaction with EC surface receptors.

### Example 16

### Depletion of Solutions from Misfolded Proteins Using Enriched IgIV

We analysed whether enriched IgIV, obtained after selection of affinity regions that bind to matrices with immobilized misfolded proteins comprising crossbeta structure, are suitable for depleting solutions from crossbeta structure. In brief, in an ELISA approach, a mixture of IgIV enriched by using Aβ fibril-Sepharose, dIgIV-Sepharose, dHSA-Sepharose and BSA-AGE-Sepharose, as described in Example 6, was immobilized, exposed to solutions with a spike of misfolded HbAGE and dOVA, and subsequently binding of the misfolded proteins to enriched IgIV was assessed.

### Materials and Method

Octagam IgIV (lot 5024018434) was enriched by using Aβ fibril-Sepharose, dIgIV-Sepharose, dHSA-Sepharose and BSA-AGE-Sepharose, as described in Example 6. The Ig concentrations were approximately 30 μg/ml. For the current experiment the four eluates from the affinity matrices were mixed 1:1:1:1 on a volume basis, and coated at a concentration of 5 μg/ml at Greiner Microlon high-binding plates, for 1 h at room temperature with motion. As a negative control buffer only or native HSA (CEALB, Sanquin, The Netherlands) was coated. ELISAs were performed essentially as described before. Blocked (Roche blocking reagent) wells coated with enriched IgIV or HSA or coat buffer were overlayed in duplicate with 0, 1, 10 or 100 μg/ml of either dOVA or HbAGE. Binding of dOVA was assayed using monoclonal anti-chicken egg albumin (Sigma, A6075, 1:10,000) and RAMPO (Dako Cytomation, P0260, 1:3,000). HbAGE was detected using an AGE specific mouse hybridoma IgG 4B5, raised against glucose-6-phosphate glycated human fibronectin, and RAMPO. Background signals obtained with buffer coated wells that were subsequently overlayed with protein solutions (see below), were subtracted from signals obtained with wells with coated enriched IgIV or HSA. In addition, background signals obtained for primary and secondary antibody incubations with wells in which no dOVA or HbAGE was added (buffer control for binding), was subtracted from signals obtained with 1, 10 and 100 μg/ml misfolded protein.

### Results and Discussion

FIG. 23 shows that dOVA is extracted from solution by immobilized enriched IgIV, whereas hardly any attachment to HSA occurred. Similarly, HbAGE was also extracted specifically by the enriched IgIV. These results show that enriched IgIV with increased affinity for misfolded proteins comprising crossbeta structure, that is immobilized on a suitable solid support, is suitable for being applied for depletion of solutions from misfolded proteins comprising crossbeta structure, like for example dOVA and HbAGE.

Applications for this disclosed method for depleting protein solutions from misfolded proteins are in the field of for example, but not restricted to, i) diagnostics for protein misfolding diseases, like for example renal failure, systemic amyloidosis, like for example AL-, AA- or ATTR amyloidosis, or RA, ii) quality control of protein solutions, like for example biopharmaceuticals and vaccines, iii) dialysis, using for example extracorporal devices, of patients suffering from protein misfolding diseases like for example renal failure, systemic amyloidosis, like for example AL-, AA- or ATTR amyloidosis, or RA, and iv) clearance of biopharmaceuticals from misfolded proteins bearing a risk for induction of (immunogenic) side effects. For all of the above mentioned applications, the specifications of the applied affinity regions with respect to preferential and specific binding to misfolded proteins, are adjusted to one's needs. In one preferred embodiment, with the methods and means described in Example 6 and 7 and the “Summary based on Examples 1-20”, given below, those specific affinity regions are selected from a composition of affinity regions, that are required for certain aimed purposes like for example those listed above.

### Example 17

### Immunomodulation of Cellular Responses to Misfolded Proteins by Enriched Affinity Regions

In order to clear the body from misfolded proteins immune cells respond to misfolded proteins in various ways. Responses include the opsonization of misfolded proteins, the production of cytokines and chemokines in order to activate and attract other cells of the immune system and the expression of cell surface markers to activate other cells. In particular, antibodies, such as affinity regions capable of specifically binding misfolded proteins, interact with immune cells in order to activate such immune cells. We tested whether affinity regions, enriched for antibodies recognizing misfolded proteins, such as glycated BSA, were able to enhance the response to misfolded proteins. We used primary human dendritic cells (DCs) isolated from peripheral blood of a healthy volunteer. We determined the production of cytokine interleukin-6 (IL-6) and chemokine IL-8, expression of cell surface markers (CD80, CD83, CD86 and CD40), as well as cell viability and survival (binding of 7AAD).

### Materials & Methods

### In Vitro Generation of Peripheral Blood Human Monocyte-Derived Dendritic Cells, and Analyses for Activation

Human DCs are generated from non-proliferating precursors selected from peripheral blood mononuclear cells (PBMCs), essentially by published methods (Sallustro and Lanzavecchia [1994], J. Exp. Med. 179 1109-1118). Relative abundant presence of CD1a, CD32, CD36, CD40, CD54, CD86, HLA-DR and CD206 and relative low content of CD14 positive, CD16 positive, CD64 positive, CD80 positive, CD83 positive and CD163 positive cells serve as a quality measure for the immature DCs. After obtaining the immature DCs upon stimulation with GM-CSF and IL-4, 1 ml of cell suspensions are incubated for 22 h with 50 μl of the following compounds (final concentrations), i) PBS, ii) 50 μg/ml poly-IC with 100 ng/ml TNFα, iii) 50 μg/ml BSA-AGE, iv) BSA-AGE +4.4 μg/ml enriched IgIV, v) as iv) but the cells are pre-incubated with a saturating concentration of blocking anti-CD32a antibody, vi) BSA-AGE +660 μg/ml Octagam IgIV, vii) as vi) but the cells are pre-incubated with a saturating concentration of blocking anti-CD32a antibody. The enriched IgIV is obtained by contacting Octagam IgIV with HbAGE-Sepharose and by subsequently isolation of those affinity regions that bound to the misfolded protein-matrix.

The DCs were analyzed for the following parameters: surface density (mean fluorescent intensity, MFI, or % positive cells) of CD83, CD86, CD80 and CD40 measured using FACS, as wells as cell death/cell viability, as determined by apoptosis marker 7-Amino-Actinomycin D (7AAD) binding. In addition, extent of IL-6 secretion and IL-8 secretion are determined in the cell culture supernatant using Pelipair ELISA (M9316, Sanquin Reagents, Amsterdam, The Netherlands) for IL-6 and a Cytosets CHC1304 kit (Biosource) for IL-8.

### Results and Discussion

Table 10 shows the results from the analysis. It is seen that DCs are potently responding to control stimulus (poly I-C in the presence of TNFalpha). The data demonstrate that enriched IgIV is able to stimulate DCs in the presence of BSA-AGE. In contrast, non-enriched IgIV at 150-fold higher concentration is hardly able to potentiate DCs. For example, the expression of IL-6 (4433 pg/ml) and IL-8 (19316 pg/ml) is potently stimulated by enriched IgIV, but to only a limited extent with non-enriched IgIV (191 pg/ml and 4682 pg/ml), respectively. In addition, enriched IgIV also stimulates the expression of co-stimulatory molecules, like CD80, CD83, CD86 and CD40. The response is inhibited by antibodies directed against FcγRIIa (anti-CD32a), indicating that the effects are mediated by this Fc receptor.

Taken together, these results show that affinity regions, preferably enriched affinity regions, serve a role in potentiating the immune system in order to remove misfolded proteins, notably through FcR. Thus, by means of the disclosed method a person skilled in the art is capable of selecting affinity regions to be used, preferably in the treatment of a disease, to remove misfolded proteins, to diminish the contribution of the misfolded proteins in the pathology of the disease.

### Example 18

### Analysis for the Presence of Anti-Cyclic Citrullinated Peptide Antibodies in Enriched IgIV Affinity Regions and in IgIV from which Enriched IgIV was Selected Using HbAGE-Sepharose Misfolded Protein Affinity Matrix

In Examples 1 and 3-9 we demonstrated that various preparations of affinity regions, i.e. human IgIV, are capable of specifically binding to misfolded proteins with crossbeta structure. In Example 10 we demonstrated that the widespread accepted method of aggregating by heating at 65° C. for preparation of human IgG for use in assays for analysis of Rheumatoid factor (RF) titers, auto-antibodies directed against the Fc domain of IgG molecules, induces crossbeta structure in the IgG molecules. RF titers are found in 70-80% of all rheumatoid arthritis patients. In addition, approximately 5% of the apparently healthy population also tests positive for RF. We now addressed the possibility that the Ig sub-population in IgIV that is capable of specifically binding to crossbeta structure or crossbeta structure-induced protein conformation has affinity for cyclic citrullinated peptide (CCP).

It has been extensively described that a population of auto-antibodies found in over 80% of rheumatoid arthritis patients, target deiminated forms of certain proteins such as fibrinogen, filaggrin and vimentin. Recently, it has been described that anti-synthetic citrullinated filaggrin sequences antibodies in fact bind to citrullinated fibrin in patients. We showed before that fibrin bears crossbeta structure conformation. In deimination, the amino acid arginine is converted to the amino acid citrulline. Therefore, this process is referred to as citrullination, resulting in citrullinated proteins. Diagnostic tests for rheumatoid arthritis are routinely used that are based on the binding of these anti-citrullinated protein auto-antibodies to citrullinated proteins, such as the anti cyclic citrullinated peptide (CCP) ELISA test (anti-CCP ELISA). It was up till the present invention that it was largely unknown how citrullination of proteins provokes an auto-immune response in RA patients. We noticed that a well-documented result of citrullination of a protein is the unfolding/refolding of the protein. According to the invention, the citrullination of arginine residues by the enzyme peptidylarginine deiminase induces misfolding of the protein comprising the arginine residue. The result of arginine citrullination is the net loss of a positive charge on the protein. This net loss of positive charge contributes to misfolding by modulation of ionic interactions and hydrogen bonds, involved in the stability and integrity of the protein three-dimensional structure. We have demonstrated previously that misfolding of proteins with the occurrence of crossbeta conformation turns the protein into an immunogenic entity (see patent application “crossbeta adjuvation”, WO2007008070). We therefore now conclude that the citrullination of proteins and the resulting misfolding of these proteins is accompanied by the formation of crossbeta structure, explaining the (auto-)immunogenic features of these citrullinated proteins. To substantiate this conclusion, we tested the presence of anti-CCP antibodies in our enriched IgIV affinity regions population that was retrieved by contacting Octagam IgIV with misfolded glycated haemoglobin, immobilized on NHS-Sepharose.

### Materials & Methods

The following affinity region preparations were analyzed for the occurrence of anti-CCP antibody titers:
1. Octagam IgIV (Octapharma, charge nr: 5024018434, 50 mg/ml)
2. 10 mg/ml human γ-globulins (Sigma G4386, Lot 21k7600). Dissolved in PBS, incubated for 10 minutes at room temperature on a roller device, and subsequently for 10 minutes at 37° C. and again for 10 minutes at room temperature on a roller device.
3. Gammagard IgIV (Baxter Hyland Immuo Gammagard S/D 5g, Lot LE08E044AL, 52 mg/ml, dissolved in supplied solution, aliquoted and stored at −20° C.).
4. 103 μg/ml enriched IgIV in PBS. Enriched from Octagam IgIV (charge nr: 5024018434) using HbAGE-Sepharose, as described in Example 6, 7.

Routine titer determinations were performed by the Laboratory for Medical Immunology (UMC Utrecht, The Netherlands) using the EliA system (Phadia GmbH) for the anti-CCP antibody titer determination. Samples 1-4 were diluted 10× for the analysis, in stead of the 100× dilution that is performed routinely for serum of patients.

### Results & Discussion

Anti-CCP antibody titers in various affinity regions preparations were determined by the local Laboratory for Medical Immunology (UMC Utrecht, The Netherlands) using the EliA system. See Table 11 for the determined titers. The values obtained with IgIV and γ-globulins preparations fall within the limits set for designating an anti-CCP titer in serum as negative with respect to the purpose of diagnosing a disease, i.e. <7 U/ml. In fact, the measured titers are regularly found in sera of apparently healthy individuals. With enriched IgIV, now, the obtained titer of 2.7 U/ml is comparable to what has been measured with Octagam IgIV, from which enriched IgIV was isolated. The concentration of enriched IgIV, however, is 485-fold lower, implicating a 437-fold enrichment of the enriched IgIV affinity regions preparation for anti-CCP antibodies. From this, we conclude that affinity regions selected based on their affinity for misfolded Hb also exhibit affinity for citrullinated peptide.

Peptidylarginine deiminase have been localized at the protein level and at the mRNA level in a wide variety of tissues and cells, but not in erythrocytes. Moreover, presence of peptidylarginine deiminases in the erythrocyte proteome was not detected in a proteomics approach. Therefore, based on these findings we conclude that the human haemoglobin (Hb) used for extensive glycation at lysine and arginine residues is not citrullinated. In addition, the used cyclic citrullinated peptides in the anti-CCP titer analysis are modified sequences based on human filaggrin and do therefore not comprise Hb amino-acid sequences. A sequence alignment with human filaggrin amino acid sequence and human Hb α-chain or β-chain amino-acid sequence does reveal low to no sequence homology (i.e. approximately 20-35%) between peptide strands of approximately 19 amino-acid residues, i.e. the length of the CCP of the second generation used in the analysis. As mentioned before, citrullination is well known for the induction of protein refolding. Therefore, our results demonstrate that with the use of a misfolded protein that comprises crossbeta structure, i.e. HbAGE, we were able to select from a collection of IgIV affinity regions a set of affinity regions with specificity for CCP, which has an amino-acid sequence that is unrelated to human Hb. With this finding we substantiate our conclusion that the misfolding, either induced by glycation, or induced by citrullination, or induced by any other means or methods for protein misfolding, results in the adoption of a common structural feature in the protein, i.e. the crossbeta structure and/or a crossbeta structure induced conformation, which is independent of the amino-acid sequence. This has an important implication for the interpretation of anti-CCP titer data. Now that it has been disclosed that affinity regions that are capable of specifically binding to citrullinated proteins comprise in fact a population of affinity regions with specificity for amino-acid sequence-independent structural features that are induced upon citrullination of the protein, implication of protein misfolding in the pathology of the diseases from which the patients with the identified anti-CCP titers suffer, can obviously not be neglected. Misfolded proteins formed through citrullination are therefore a newly identified target for the direction of the research conducted to the development of, for example, RA specific therapies. Our results, now, demonstrate that the enriched IgIV affinity regions obtained using a misfolded protein-matrix, are such a newly identified lead compound for drug development against RA-related misfolded proteins.

### Example 19

### Human Enriched IgIV Affinity Regions with Specificity for Misfolded Mouse γ-Globulins

### Background

Rheumatoid Factor (RF) is a composition of IgA, IgG, IgM auto-antibodies directed to epitopes in the Fc domain of self-IgG molecules, that are exposed upon misfolding of the IgG by exposure to heat. RF occurs in 70-80% of rheumatoid arthritis (RA) patients, and relatively high RF titers correlate with severe disease progression. In Example 10, we demonstrate that methods to expose the RF epitope in fact misfold the IgG's in a way that crossbeta structure is formed, resulting to the conclusion that RF are affinity regions with affinity for crossbeta structure or crossbeta structure induced conformation in IgG. We found that immunization of a mouse with four different proteins with crossbeta structure, i.e. synthetic human Aβ1-40, chicken serum amyloid A, glycated human haemoglobin and synthetic fragment of human fibrin α-chain, elicited an immune response resulting in a hybridoma IgM clone with specificity for misfolded human IgG, that comprises crossbeta structure. Either one, or more of the four protein antigens with unrelated amino-acid sequences but with the presence of crossbeta structure or crossbeta structure induced conformations in common, comprise crossbeta structure or crossbeta structure induced structural features that is by chance closely reminiscent to the crossbeta features in misfolded human IgG. An alternative explanation is that structural crossbeta features in one or more of the four antigens resembles crossbeta structure or crossbeta structure-induced conformation in a mouse self-Ig molecule. Cross-reactivity may have occurred during high activity of the immune system, accompanied by over-production of Ig's by β-cells. Abnormal reactivity of the mouse immune system is concluded from the extremely large spleen (seven-fold increased number of cells), accompanied by a large number of infiltrated fibroblasts. Moreover, the mouse was critically ill for a while during the immunization trial. These observations may be the concequence of an auto-immune response against self-IgG, reflected in the observed affinity of the hybridoma IgM for misfolded human IgG. A third plausible explanation is that the mouse just had a general crossbeta binding IgM clone with properties in common with RF in its repertoire, resembling the IgG's that are selected from human IgIV by applying a crossbeta affinity matrix. We now assessed whether human IgIV that is enriched for affinity regions with affinity for misfolded proteins upon selection on an HbAGE-matrix, comprises affinity regions with specificity for misfolded mouse IgG. This will further substantiate our knowledge on the existence of a population of self-immunoglobulins with specificity for misfolded proteins in general.

### Materials and Methods

To test whether Octagam IgIV starting material used as a pool for selection of affinity regions binding to crossbeta structure, and enriched human IgIV comprise a population of affinity regions with specificity for misfolded mouse IgG with crossbeta structure, we analyzed binding of Octagam IgIV and enriched IgIV to various misfolded forms of mouse IgG and compared the results with binding to native mouse IgG. Measuring ThT fluorescence and Congo red fluorescence with native mouse IgG, mouse IgG exposed to high pH (dmIgG BASE), mouse IgG exposed to low pH (dmIgG ACID) and mouse IgG heated to 85° C. in PBS (dmIgG 85° C.) revealed that crossbeta structure is induced by the various misfolding methods. The ELISA was performed in two different ways. In one approach, the mouse IgG was directly coated onto the wells and overlayed with a concentration series of enriched IgIV. In an alternative manner, first rabbit anti-mouse immunoglobulins (RAMPO, Dako Cytomation, Denmark) was coated onto the wells. Wells were blocked (Roche blocking reagent) and subsequently, the mouse IgG preparations were bound to the immobilized antibodies, before a concentration series of Octagam human IgIV was applied to the wells in triplicate.

### Results & Discussion

In FIG. 24 the results of the two alternative ELISA approaches are summarized. In both experimental approaches the human affinity regions bind preferentially to the various misfolded forms of mouse IgG. Hardly any binding of enriched IgIV to native mouse IgG is detected, and Octagam IgIV did not bind at all to native mouse IgG. Both Octagam IgIV and enriched IgIV bound with highest affinity to dmIgG BASE, with concentrations resulting in half maximum binding of approximately 200 μg/ml and 4.4 μg/ml IgIV, respectively. The fact that with enriched IgIV some binding to native mouse IgG is seen whereas no binding could be detected with Octagam IgIV points to the presence of a certain fraction of misfolded IgG molecules in the mouse IgG composition, for which enriched IgIV has increased affinity. From these figures it is deduced that enriched IgIV is enriched for binding to misfolded mouse IgG with approximately a factor 50.

In conclusion, the three different forms of misfolded mouse IgG comprise binding sites for enriched human IgIV and Octagam IgIV, from which enriched IgIV is selected. dmIgG BASE exposes misfolded protein conformation for which both Octagam IgIV and enriched IgIV express highest affinity. These data show that by using an affinity matrix composed of misfolded glycated hemoglobin, a population of affinity regions is selected from a composition of IgG molecules, i.e. Octagam IgIV, that exhibits affinity for misfolded mouse IgG. This points to the occurrence of RF like affinity regions in the selected enriched IgIV fraction, and thus in the Octagam IgIV, i.e. affinity regions that preferentially bind to misfolded affinity regions with crossbeta structure.

### Example 20

### A Hybridoma IgM with Binding Properties Reminiscent to Rheumatoid Factor

### Background

As mentioned before in the Materials and Methods section to Examples 1-5, mouse hybridoma IgM 7H2H2 binds specifically to some misfolded forms of human immunoglobulins. We therefore designated 7H2H2 as a Rheumatoid Factor like antibody. The mouse was immunized consecutively with synthetic human Aβ1-40, chicken serum amyloid A, glycated human haemoglobin and synthetic peptide of human fibrin α-chain, before the spleen was isolated for preparation of hybridoma's. Noteworthy, at the time the spleen was removed, it comprised an extraordinary large number of cells, 7*10⁸ (normal number is 1*10⁸ cells). In addition, the spleen comprised an exceptionally high number of infiltrated fibroblasts. These observations point to a highly active spleen, due to high activity of the mouse immune system. Noteworthy, approximately 40 weeks after the first immunizations with Aβ, before any immunization with a second, third or fourth misfolded antigen, the mouse got ill, but recovered within a few weeks, well before the immunization with the second antigen, i.e. chicken SAA. The fact that 7H2H2 recognizes γ-immunoglobulins and misfolded IgIV, whereas four antigens are used for immunizations that comprise unrelated amino-acid sequences, and no (foreign) immunoglobulins are used as antigen, combined with the observation of a highly activated immune system and the illness of the mouse at some point during the immunization procedure, let us to conclude that the mouse developed an auto-immune response directed to self-antibodies. To further analyse the structural requirements of human IgG's in order to expose the epitope for 7H2H2, we performed binding experiments with a series of human IgG preparations that comprise misfolded antibodies obtained through different methods.

### Materials & Methods

For the analysis of the binding of hybridoma clone 7H2H2 IgM to various structure appearances of human IgG, a dilution series of purified 7H2H2 (2.5 mg/ml in PBS; P. van Kooten, ABC-Hybridoma-facility, University of Utrecht/UMC Utrecht, The Netherlands) or a fixed concentration of 12.5 μg/ml IgM was used in ELISAs with immobilized human IgG's. As a negative control, hybridoma IgM 2G10 was used. Misfolded forms of human IgG's and native controls used for the analyses are depicted in FIG. 25, and are: 1) IgIV 5 minutes at 65° C. (‘RF’ method), 2) IgIV 65° C., 3) IgIV 69, 4) IgIV 76, 5) IgIV 80, 6) IgIV 83, 7) IgIV 86, 8) IgIV Acid/Base control, 9) IgIV Acid, 10) IgIV Base, 11) native Gammagard IgIV, 12) IgIV HFIP/TFA, 13) IgIV NaPi 5 mg/ml, 14) IgIV NaPi 20 mg/ml, and 15) IgG Base denatured, 37° C. For structure details, refer to the ‘General Materials and Methods for Examples 6-20’ section on preparation and structure determination of crossbeta standards. In FIGS. 8 and 9, structural features of the 15 forms of human IgG are depicted. The human γ-globulins that were warmed for 30 minutes at 37° C. after adding NaOH (hIgG-BASE-37° C.) appear as large particulates in suspension (FIG. 9L), displays increased Trp fluorescence when compared to native IgIV (FIG. 8F) and the preparation enhances ThT and CR fluorescence (FIG. 8A, B).

In a second experiment, binding of a concentration series of purified mouse hybridoma IgM 7H2H2 to various preparations of mouse γ-globulins was compared to binding to hIgG-BASE-37° C. and control native IgIV Gammagard, and compared to binding of control mouse hybridoma IgM 2G10 to the same series of IgG preparations. The same mouse IgG preparations as in Example 19 were incorporated in the study, i.e. native mIgG, dmIgG ACID, dmIgG BASE, dmIgG 85° C. The mouse and human IgG preparations at 5 μg/ml or control buffer was coated on Microlon high-binding ELISA plates (Greiner), which were blocked with Blocking reagent (Roche) after coating. IgM 7H2H2 and IgM 2G10 (negative control) were applied to the wells in triplicate at 0/1/10/100 μg/ml in PBS/0.1% Tween20. After washing, binding of IgM was detected using secondary goat anti-mouse-IgM-PO antibody (Jackson), diluted 1:5000 in PBS/0.1% Tween20. Absorbance was read at 450 nm. Background signals measured for non-coated wells with the concentration series of IgM, and background signals obtained with IgG coated wells with 0 μg/ml IgM, but with secondary antibody, were subtracted from corresponding signals with IgG coated wells overlayed with IgM.

### Results & Discussion

In FIG. 25A it is depicted that 12.5 μg/ml of mouse hybridoma IgM 7H2H2 does not or hardly bind to human IgG preparations 1, 2, 3, 9, 10, 11, 13 and 14, binds to a little extent to preparations 4 and 5, binds moderately to 6, 7, 8 and 12, and binds best to human IgG preparation 15 (γ-globulins, basic conditions, treated for 30 minutes at 37° C., followed by pH adjustment with HCl back to physiological pH). When binding of the purified 7H2H2 is analysed with preparations 1, 6, 11, 14 and 15, no binding to native Octagam IgIV, 1) IgIV 5 minutes at 65° C. (‘RF’ method), 11) native Gammagard IgIV or 14) IgIV NaPi 20 mg/ml is detected (FIG. 25B). Similarly high-affinity binding is seen with human IgG preparation 6) Gammagard IgIV heated to 83° C. at 5 mg/ml in 20 mM sodium phosphate pH 5.0, and 15) IgG Base denatured, 37° C., at 5 mg/ml, at 37° C. Both the number of binding sites is comparable (Bmax is 0.65 and 0.59 a.u., respectively), as well as the concentration 7H2H2 at which half of the binding sites are occupied, i.e. 3.3 μg/ml 7H2H2 for both immobilized misfolded IgG preparations. Preparation 15) appeared on TEM images as aggregate structures similar to IgIV BASE (sample 10) and IgIV HFIP/TFA (sample 12). The negative control for IgM binding to the immobilized human IgG's, hybridoma IgM 2G10, did not show any affinity for the human IgG preparations (data not shown). For preparation 6) it is evident from FIG. 9 that all fluorescent probes bind to a relatively high extent, and even to the highest extent for Congo red and Thioflavin S, compared to all other preparations (FIG. 8). However, sample 6) moderately enhanced tPA/plasminogen activation, whereas 9) and 12) strongly potentiated the protease activity (FIG. 9M). Analysis of TEM images revealed that increase in fluorescence of dyes to some extent positively correlates with an increase in multimer size. It is concluded that the six fluorescence data points (CR, ThT, ThS, Trp, bis-ANS and ANS) altogether build up predictive power for the expected binding of 7H2H2. Multiplication of the signals for each IgG preparation would indeed predict that sample 6) will display as the best suitable ligand for the hybridoma clone. When tPA/plasminogen activation is also considered, somewhat higher binding of 7H2H2 to samples 9) and 12) is predicted. In conclusion, it appears that the increased magnitude of binding of a series of fluorescent dyes with affinity for crossbeta structure, i.e. CR, ThT and ThS, or that probe solvent-exposure of hydrophobic patches in the protein structure, i.e. bis-ANS and ANS, and changes in the local environment of Trp residues, displayed as increases in fluorescence intensity, predict whether the hybridoma IgM clone 7H2H2 will bind with high affinity. These results clearly demonstrate that the mouse at some moment, either as an innate immune response, or as an adaptive response upon exposure to one or more of the four foreign crossbeta antigens used for immunization, developed an immune response to epitopes that are hidden or not present in natively folded IgG's, i.e. exposed crossbeta structure, or crossbeta structure induced conformation. In summary, our results show that by choosing a certain misfolded protein or set of misfolded proteins, an immune response in mice is inflicted resulting in affinity regions with clear specificity for a defined misfolded protein, with preferential binding to a certain appearance of the crossbeta structure or crossbeta mediated exposed conformation.

In FIG. 25C it is shown that 7H2H2 at all tested concentrations binds to hIgG-BASE-37° C., in accordance to what has been demonstrated in FIGS. 25A and B. At 100 μg/ml also some binding to native IgIV Gammagard is seen. This may reflect the presence of a certain percentage of IgIV aggregates in Gammagard, or this may display the denaturing conditions of the used ELISA plate. The negative control IgM 2G10 did not bind at all. In FIG. 25D it is seen that already at 1 μg/ml 7H2H2 binds to acid-denatured mouse γ-globulins (dmIgG-ACID). At 10 and 100 μg/ml the hybridoma IgM binds to all three forms of misfolded self-IgG, with largest signals obtained at 100 μg/ml for dmIgG-ACID and dmIgG-BASE. At 100 μg/ml also some binding to native mIgG occurs. The negative control IgM 2G10 did not bind at all to any of the mouse IgG preparations (not shown). With these results it is clearly demonstrated that the hybridoma mouse IgM 7H2H2 not only binds specifically to misfolded forms of human IgG, but also to misfolded forms of mouse self-IgG. This shows that the mouse from which the hybridoma clone 7H2H2 was selected developed an auto-immune response against self-IgG. This may have occurred during the immunization trials with the four different non-IgG, non-self misfolded proteins, i.e. human synthetic Aβ, chicken SAA, human HbAGE and synthetic human fibrin fragment. With the observation that 7H2H2 binds to misfolded mouse self-IgG with crossbeta structure, this hybridoma IgM is designated as a Rheumatoid Factor antibody. Illness of the mouse during the immunization experiment and the unusual large spleen with an unusual large amount of infiltrated fibroblasts is related to a triggered auto-immune response while immunized with different misfolded proteins comprising crossbeta structure.

### Recombinant/Synthetic Affinity Regions

Generation of Recombinant/Synthetic Affinity Regions Obtained from Enriched IgIV

The present invention discloses methods and means for the selection of affinity regions specific for misfolded proteins for the diagnosis and treatment of protein misfolding and protein misfolding diseases. Affinity regions are selected from any combinatorial library of affinity regions, such as for example natural occurring human immunoglobulins (i.e. human IVIg or IgIV). Affinity regions analogous as those obtained in this way are for instance made recombinantly or synthetically by applying standard techniques, known to a person skilled in the art, including protein sequence analysis, DNA cloning and expression technology. This example describes one embodiment. In subsequent steps: (1) The amino acid sequence, at least from the variable regions of both heavy and light chains, or at least from the complementarity determining regions 1-3 (CDRs), or at least from CDR3 of the heavy chain (HC) of the individual isolated affinity regions, is obtained by protein sequence analysis. (2) A DNA sequence encoding the identified amino acids sequence is made synthetically. As an alternative to the exact sequence determined by protein analysis, a sequence can be used wherein one or more mutations are introduced, preferably in the CDR3, and even more preferably in the CDR3 of the heavy chain (HC), in order to produce affinity regions with altered affinity, preferably increased and/or more specific affinity. (3) The DNA is cloned into an appropriate expression vector. Such vector preferably already contains the sequences encoding the constant regions of immunoglobulins of the desired type, such as to obtain IgG1, IgG2a, IgG2b, IgM, IgA, IgE etc. (4) The vector is transduced in either way into an expression system of choice, preferably a mammalian cell. (5) The cells expressing the affinity region are selected. (6) Recombinantly made affinity regions are purified from the cells or cell derived culture supernatant. If mutations are introduced into the original affinity region sequence to optimize affinity, the newly made affinity regions can be re-selected using the disclosed methods and means. Such generation of semi-synthetic affinity regions with an even increased repertoire of affinity regions, preferably in the complementarity determining regions, preferably in the CDR3, even more preferably in the CDR3 of the HC, is preferably performed by generation of a semi-synthetic library, such as a phage display library (see below).

### Generation of Recombinant/Synthetic Affinity Regions

Besides a collection of human immunoglobulins such as IVIg obtained from blood, a combinatorial library can also be obtained from any other set of affinity regions, preferably a set of recombinant affinity regions such as those present in a phage display library (Winter et al. 1994; Hoogenboom, 1992, 1997, 2000, 2002, 2005). Preferably, such a library is comprised of sequences related to mammalian affinity regions, preferably human affinity regions, like immunoglobulins. Preferably, such a phage display library comprising a collection of affinity regions is made as follows (Winter et al. 1994, de Kruif et al. 1995a, 1995b). First RNA is extracted from B cells or from a tissue comprising B cells. Subsequently, cDNA is prepared. Next, cDNA encoding the variable regions is amplified, cloned into an appropriate phagemid vector and transformed into an appropriate host, such as for example a strain of Escherichia coli. In this way affinity regions are expressed, i.e. displayed by phages, as fusion proteins on the surface of filamentous bacteriophages. A phage display library is for instance prepared from B cells obtained from a healthy mammal, preferably a human, mouse, rat or llama, or alternatively from a mammal immunized with a misfolded protein. In one embodiment, a phage display library is prepared from B cells from a mammal, preferably a human suffering from a particular disease, preferably a misfolding disease, like for example RA. In this way, a collection of affinity regions is prepared with a specific aim to comprise those affinity regions specific for misfolded proteins. For example a mouse is immunized once or several times with one or a selection of misfolded proteins (like in this Example 20), B cells are isolated from the spleen and used to prepare a phage display library. In another example, B cells are isolated from a human with a particular disease, for example (rheumatoid) arthritis. cDNA prepared from these B cells is then used to prepare a phage display library. In such a way a phage display library is prepared to comprise affinity regions with specificity for misfolded proteins involved in the chosen misfolding disease. For example, a library is prepared with affinity regions for the Fc domain of Ig's, i.e. affinity regions like Rheumatoid Factor (RF) (van Esch et al. 2003, Clin Exp. Immunol). In the above described way a person skilled in the art is able to design and prepare a phage display library with any collection of affinity regions with emphasis on a particular disease or application.

A phage display library with such a collection of affinity regions with an increased repertoire is also prepared synthetically (Hoogenboom, 1992, 1997, 2000, 2002, 2005; de Kruif et al. 1995a, 1995b). In this way a person skilled in the art is able to design a library comprising affinity regions of considerable additional diversity. Most notably, by implementing additional sequences in the hypervariable regions, the CDRs that interact with the antigen, additional affinity regions are made, reshaping the variable domains. Besides affinity regions obtained from human sequences, a person skilled in the art is able to create a collection of affinity regions from any other species, such as llama, camel, alpaca or camelid, to obtain affinity regions, such as llama antibodies, also referred to as nanobodies, with properties related to these species. Thus, a phage display library and/or a collection of affinity regions is prepared in many ways, preferably from a mammal immunized with one or a set of misfolded proteins. In a particularly preferred embodiment, a phage display library and/or a collection of affinity regions is prepared from a mammal with a disease, preferably a misfolding disease. Affinity regions specific for misfolded proteins are selected from a phage display library using the disclosed means and methods, combined with standard procedures for isolating phages. Most straightforward, in a preferred embodiment, misfolded proteins are prepared and are immobilized, preferably according to any one of the procedures disclosed in this application, and subsequently allowed to bind phages. After extensive washing bound phages are retrieved and amplified by reinfection of host. To allow recovery of only specific phages the selection procedure is preferably repeated several times. Finally, those phages are isolated that are capable of specifically binding misfolded targets. Alternatively, misfolded proteins are isolated from a tissue sample obtained from an individual or combination of individuals with a disease. For example, misfolded proteins are isolated using a protein that is capable of specifically binding to misfolded proteins comprising crossbeta structure, such as tPA, RAGE or a functional equivalent thereof (see Table 4), from synovial fluid of a patient with (rheumatoid) arthritis. In analogy, any other sample can be taken.

Using approaches as described above recombinantly made affinity regions for misfolded proteins are obtained.

After selection of the appropriate phages DNA encoding the variable regions of the isolated affinity regions are preferably isolated from the phagemid DNA in order to generate full antibodies. This is easily performed by a person skilled in the art according to standard procedures. The DNA is preferably cloned into vectors encoding the constant regions for the heavy and light chains. Any vector can be used and any desired type of constant region. The vector is transduced in any known way into an expression system of choice, preferably a mammalian cell. The cells expressing the affinity region are selected. Recombinantly made affinity regions are preferably purified from the cells or cell derived culture supernatant. In such a way any immunoglobulin affinity region for misfolded proteins is prepared (Bloemendal et al 2004; Huls et al 1999a, 1999b; Boel et al 2000).

### Generation of “Chimeric” or “Humanized” Recombinant Affinity Regions

For use in humans, affinity regions obtained from other species are preferably modified in such a way that non-human sequences are replaced with human sequences, wherever possible, while preferably not too much influencing the binding properties of the affinity region. Affinity regions are also made during classical immunization strategies, preferably using mice or rats, even more preferably using transgenic mice that encode human immunoglobulins. After immunization hybridoma cell lines expressing monoclonal antibodies are prepared by standard procedures, or by applying the above described phage display technology. Monoclonal antibodies are selected that are capable of specifically interacting with misfolded proteins. “Chimeric” or “humanized” versions of such affinity regions, when made using normal mice or rats, are for instance made by replacing the non-human constant regions and the relevant non-human variable regions with the relevant human homologous regions (Morrison et al 1984; Jones et al. 1986). Moreover, different constant regions are introduced when desired.

### Summary Based on Examples 1-20

Procedure to Select Affinity Regions Enriched for One or a Set of Misfolded Proteins, Preferably Specific for a Particular Disease or Health Problem Associated with the Misfolded Protein or Set of Misfolded Proteins.

In Examples 1 to 9 we demonstrated that with the use of affinity matrices containing misfolded proteins comprising misfolded proteins and/or crossbeta structure, affinity regions are selected from any composition of affinity regions, that preferentially and selectively and with increased affinity bind to misfolded proteins and/or proteins comprising crossbeta structure, that were not necessarily included in the set of affinity regions used for the selection. The Examples demonstrated that with the use of a solid support with immobilized selected misfolded proteins comprising crossbeta structure, we are able to isolate from a collection of affinity regions those affinity regions which have affinity for virtually any misfolded protein. With HbAGE-, dHSA-, Aβ fibril- and dIgIV-matrices we selected affinity regions that bind to dHSA, Aβ fibrils, Aβ non-fibrillar aggregates, dOVA, BSA-AGE, Hb-AGE, misfolded mouse IgG, citrullinated peptide/protein, ApoA-I and oxLDL. Multiple members of this list of ligands for the enriched IgIV composition contribute to the pathology of protein misfolding diseases, like for example Aβ (Alzheimer's disease), oxLDL and ApoA-I (atherosclerosis, amyloidosis), glycated proteins (amyloidosis, end-stage renal disease, diabetes, RA), misfolded IgG, citrullinated proteins (AL amyloidosis, RA).

In addition, we showed that both with misfolded proteins with fibrillar appearance, as well as misfolded protein aggregates lacking fibrillar features, affinity regions are selected which exhibit broad range specificity for misfolded proteins comprising crossbeta structure. With Aβ fibril-affinity matrix affinity regions were selected that displayed affinity for non-fibrillar multimers of for example misfolded BSA-AGE, aggregates of Aβ and dOVA. At the other hand, with the use of non-fibrillar HbAGE-matrix or non-fibrillar misfolded IgIV-matrix, affinity regions were selected that efficiently binds to Aβ fibrils.

With the use of a bovine serum albumin-AGE-matrix, affinity regions with affinity for human Aβ, human albumin and chicken ovalbumin was demonstrated. With the use of human Aβ-matrix affinity regions that bind to glycated bovine serum albumin and chicken ovalbumin were selected. With glycated human Hb-matrix affinity regions binding to misfolded mouse IgG were selected. These data show that with misfolded proteins originating from one species, human affinity regions are selected that have affinity for misfolded proteins originating from other species.

In conclusion, we demonstrated that from a collection of human IgIV affinity regions a selection of affinity regions originating from at least four different β-cell clones producing IgG1, IgG2, IgG3 and IgG4 iso-types, was selected that exhibit binding properties towards a wide range of proteins originating from various species and that have neither substantial amino-acid sequence homology, nor similar amino acid sequence length, nor overlapping or similar 3D structure in their native fold, though that share a structural feature common to misfolded proteins. This structural feature can be introduced in the protein structure by various means, like for example but by no means restricted to glycation of lysine and arginine residues, citrullination of arginines, oxidation of amino acid side chains, and any combination of exposure to low pH, high pH, heat, carbohydrates, all at varying protein concentration. The selected affinity regions with specificity for misfolded proteins and/or proteins comprising crossbeta structure are useful for a variety of applications. Below, enriched affinity regions used for therapy against protein misfolding diseases is outlined in more detail.

The disclosed means and methods allow for the selection of affinity regions that are applicable in therapeutics and/or diagnostics for diseases associated with protein misfolding. A summary outlining the general characteristics of preferred procedures is depicted in FIG. 26. Any misfolded protein of choice (mix X and Y in FIG. 26, representing the Misfoldome) is suitable for being used to select affinity regions, but preferably misfolded proteins (mix A in FIG. 26) are used that are implicated in disease. Since misfolded proteins share common characteristics, in general, affinity regions will be selected that bind to more than one particular misfolded protein. However, as disclosed in this application, also affinity regions can be selected that preferentially bind a subset or even a single type of misfolded protein. By combining a set of columns a person skilled in the art is able to select those affinity regions that are applicable for therapeutics and/or diagnostics for misfolding in general or that are preferentially applicable for a particular disease or set of diseases in which a misfolded protein of choice is implicated. As illustrated in FIG. 26 application of column I (mix of misfolded proteins not necessarily related to a disease) will result in affinity regions (preparation 1) with affinity for misfolded proteins in general, i.e. the Misfoldome. Such affinity regions is suitable for use for diagnostics and also for therapy. However use of such affinity regions for therapeutic purposes implies the potential risk for side effects, due to the fact that affinity regions are introduced to the patient that not only bind to the disease-related misfolded protein (desired therapeutic effects), but in addition to other misfolded proteins present (unpredictable side-effects of the therapy). By combining columns I and III, and more preferably II and IV a person skilled in the art selects those affinity regions that preferentially interact with misfolded proteins specific for a disease or a set of diseases. Column IV is used to remove those affinity regions that are capable of interacting with misfolded proteins which are not related to the target disease of choice. Hence, preparations 3 and 4 are preferentially selected for specific therapeutic purposes.

### Tables:

TABLE 1
reported side effects related to administering IgIV to patients‡

| | | | |
|---|---|---|---|
| Venous thrombosis | arterial thrombosis | Headache | chills |
| nausea | fever | Cramping | tachycardia |
| aseptic meningitis | (acute) renal failure | Anaphylaxis | thromboembolic events |
| Pseudohyponatremia | back pain | passagere headache | seizures |
| hypotension | haemolytic anaemia | haemolytic hemolysis | nephro-toxicity |
| intolerance (anti-IgA | Pseudohyponatraemia | reduced immune | Acquired von |
| antibodies when IgA | in newborn | response against | Willebrand's syndrome |
| deficient | | some living virus | in association with a |
| | | vaccines (mumps, | lupus-like anticoagulant |
| | | measles, | |
| | | varicella/rubella | |
| | | vaccine) | |
| exanthema | eczema | pure red cell aplasia | fatigue |
| cerebrovascular | Hyperviscosity in | Acute myocardial | transient ischaemic |
| accidents | newborn | ischemia | attacks |
| Transient neutropenia | Acute renal transplant | Acute myocardial | Hemolytic uremic |
| | injury | infarction | syndrome |
| pain at injection site | | | |

‡Data is retrieved from literature references obtained by Pubmed data mining, and from Octagam and Gammagard datasheets.

TABLE 2
Sequence identities of synthetic peptides

| | Sequence | |
|---|---|---|
| peptide | identity | Amino-acid sequence |
| FP13 K157G | SEQ-ID 1 | KRLEVDIDIGIRS |
| Aβ(1-40) | SEQ-ID 2 | DAEFRHDSGYEVHHQKLVFFAEDVGSNKGAIIGLMVGGVV |
| Aβ(1-40) E22Q | SEQ-ID 3 | DAEFRHDSGYEVHHQKLVFFAQDVGSNKGAIIGLMVGGVV |
| FP10 | SEQ-ID 4 | KRLEVDIDIK |
| Yeast prion | SEQ-ID 5 | GNNQQNY |
| peptide | | |
| FP6 | SEQ-ID 6 | IDIKIR |
| TRAP | SEQ-ID 7 | SFLLRN |
| PPACK | SEQ-ID 8 | FPR-chloromethylketone |
| Abeta1-42 | SEQ-ID 9 | DAEFRHDSG YEVHHQKLVF FAEDVGSNKG AIIGLMVGGV VIA |

TABLE 3
cross-β structure conformation binding compounds

| Congo red | Chrysamine G | Thioflavin T |
|---|---|---|
| 2-(4′-(methylamino)phenyl)- | Any other amyloid- | Glycosamino- |
| 6-methylbenzothiaziole | binding dye/chemical | glycans |
| Thioflavin S | Styryl dyes | BTA-1 |
| Poly(thiophene acetic | conjugated polyeclectro- | |
| acid) | lyte PTAA-Li | |

TABLE 4
proteins that act in the Crossbeta Pathway by binding to and/or interacting with misfolded proteins

| | | |
|---|---|---|
| Tissue-type plasminogen activator | Finger domain(s) of tPA, factor XII, | Apolipoprotein E |
| | fibronectin, HGFA | |
| Finger domains | Proteins comprising finger domains, e.g. | Affinity regions |
| | tPA, HGFA, factor XII, fibronectin | |
| Factor XII | Plasmin(ogen) | Matrix metalloprotease-1 |
| Fibronectin | 75kD-neurotrophin receptor (p75NTR) | Matrix metalloprotease-2 |
| Hepatocyte growth factor activator | α2-macroglobulin | Matrix metalloprotease-3 |
| Serum amyloid P component | High molecular weight kininogen | Monoclonal antibody 2C11(F8A6)^{‡} |
| C1q | Cathepsin K | Monoclonal antibody 4A6(A7)^{‡} |
| CD36 | Matrix metalloprotease 9 | Monoclonal antibody 2E2(B3)^{‡} |
| Receptor for advanced glycation endproducts | Haem oxygenase-1 | Monoclonal antibody 7H1(C6)^{‡} |
| Scavenger receptor-A | low-density lipoprotein receptor-related | Monoclonal antibody 7H2(H2)^{‡} |
| | protein (LRP, CD91) | |
| Scavenger receptor-B | DnaK | Monoclonal antibody 7H9(B9)^{‡} |
| ER chaperone Erp57 | GroEL | Monoclonal antibody 8F2(G7)^{‡} |
| Calreticulin | VEGF165 | Monoclonal antibody 4F4^{‡} |
| Monoclonal conformational antibody WO1 (ref. | Monoclonal conformational antibody WO2 | Amyloid oligomer specific antibody |
| (O'Nuallain and Wetzel, 2002)) | (ref. (O'Nuallain and Wetzel, 2002)) | (ref. (Kayed et al., 2003)) |
| formyl peptide receptor-like 1 | α(6)β(1)-integrin | CD47 |
| Rabbit anti-albumin-AGE antibody, Aβ- | CD40 | apo A-I belonging to small high-density |
| purified^{a)} | | lipoproteins |
| apoJ/clusterin | 10 times molar excess PPACK, 10 mM | CD40-ligand |
| | εACA, (100 pM-500 nM) tPA²⁾ | |
| macrophage scavenger receptor CD163 | Affinity region with affinity for mouse d-γ- | BiP/grp78 |
| | globulins | |
| Erdj3 | haptoglobin | α2-macroglobulin-trypsin complex |
| α2-macroglobulin-α-chymotrypisin complex | α2-macroglobulin-bromelain | Rheumatoid factor |
| Rheumatoid factor IgA isotype | Rheumatoid factor IgG isotype | Rheumatoid factor IgM isotype |
| B-cell receptor with alpha, or gamma, or mu | Anti-cyclic citrullinated peptide | Anti-citrullinated protein (auto)antibody |
| chains | (auto)antibody | |
| HSP60 | HSP90 | DNAK |
| HSP104 | ClpA | ClpB |
| Affinity regions with affinity for misfolded | Anti-citrullinated protein/peptide antibody | Affinity regions collected from a |
| proteins | | composition of affinity regions |
| | | using a crossbeta affinity matrix |
| Affinity regions collected from a composition of | Affinity regions collected from a | Affinity regions collected from a |
| affinity regions using a crossbeta HbAGE | composition of affinity regions using a | composition of affinity regions using |
| affinity matrix | crossbeta dIgIV affinity matrix | a crossbeta BSSAGE affinity matrix |
| Affinity regions collected from a composition of | Affinity regions collected from a | Affinity regions collected from a |
| affinity regions using a crossbeta Aβ affinity | composition of affinity regions using a | composition of affinity regions using |
| matrix | crossbeta Aβ fibril affinity matrix | a crossbeta dHSA affinity matrix |
| broad spectrum (human) immunoglobulin G | Affinity regions with affinity for crossbeta | Affinity regions collected from patient |
| (IgG) antibodies (IgIV, IVIg) | structure or crossbeta induced | serum/plasma/synovial fluid using affini [symbol] |
| | conformation, e.g. collected from a | region matrix with affinity for crossbeta |
| | composition of affinity regions | structure and/or crossbeta induced |
| | | conformation |
| Affinity region with affinity for oxLDL/ApoB-100 | Affinity region with affinity for misfolded | Affinity region with affinity for Aβ |
| | ApoA-I | |
| Affinity region with affinity for Aβ fibril | Affinity region with affinity for non-fibrillar | Affinity region with affinity for fibrin |
| | Aβ aggregates | |
| Affinity region with affinity for HbAGE | Affinity region with affinity for BSAAGE | Affinity region with affinity for |
| | | glycated protein |
| Affinity region with affinity for citrullinated | Affinity region with affinity for dOVA | Affinity region with affinity for dHSA |
| protein | | |
| Affinity region with affinity for human dIgIV | Macrophage scavenger receptor - 1 | Anti-cyclic citrullinated peptide antibody |
| | (MSR-1) | |

^{‡}Monoclonal antibodies developed in collaboration with the ABC-Hybridoma Facility, Utrecht University, Utrecht, The Netherlands.
^{a)}Antigen albumin-AGE and ligand Aβ were send in to Davids Biotechnologie (Regensburg, Germany); a rabbit was immunized with albumin-AGE, antiboc [symbol] against a structural epitope were affinity purified using a column with immobilized Aβ.
²⁾PPACK is Phe-Pro-Arg-chloromethylketone (SEQ-ID 8), εACA is ε-amino caproic acid, tPA is tissue-type plasminogen activator [symbol]
indicates data missing or illegible when filed

TABLE 5
Proteins that are part of the Crossbeta Pathway

| | | |
|---|---|---|
| Monoclonal antibody 4B5 | Heat shock protein 27 | Heat shock protein 40 |
| Monoclonal antibody 3H7^{‡} | Nod2 (=CARD15) | Heat shock protein 70 |
| FEEL-1 | Pentraxin-3 | HDT1 |
| LOX-1 | Serum amyloid A proteins | GroES |
| MD2 | Stabilin-1 | Heat shock protein 90 |
| FEEL-2 | Stabilin-2 | CD36 and LIMPII analogous- |
| | | I (CLA-1) |
| Low Density Lipoprotein | LPS binding protein | CD14 |
| C reactive protein | CD45 | Orosomucoid |
| Integrins | alpha-1 antitrypsin | apo A-IV-Transthyretin |
| | | complex |
| Albumin | Alpha-1 acid glycoprotein | β2-glycoprotein I |
| Lysozyme | Lactoferrin | Megalin |
| Tamm-Horsfall protein | Apolipoprotein E3 | Apolipoprotein E4 |
| Toll-like receptors | (pre)kallikrein | CD11d/CD18 (subunit aD) |
| CD11b2 | CD11a/CD18 (LFA-1, subunit | CD11c/CD18 (CR4, subunit |
| | aL) | aX) |
| Von Willebrand factor | Myosin | Agrin |
| Perlecan | Chaperone60 | b2 integrin subunit |
| proteins that act in the unfolded | proteins that act in the | Macrophage receptor with |
| protein response (UPR) pathway | endoplasmic reticulum stress | collagenous structure |
| of the endoplasmic reticulum | response (ESR) pathway of | (MARCO) |
| (ER) of prokaryotic and | prokaryotic and eukaryotic | |
| eukaryotic cells | cells | |
| 20S | CHAPERONE16 family | HSC73 |
| | members | |
| HSC70 | plasmin(ogen) | 26S proteasome |
| 19S cap of the proteasome | hepatocyte growth factor/ | carboxy-terminus of |
| (PA700) | scatter factor | CHAPERONE70-interacting |
| | | protein (CHIP) |
| Pattern Recognition Receptors | Derlin-1 | Calnexin |
| Thrombospondin | GRP94 | Endoplasmic reticulum p72 |
| (broad spectrum) (human) | proteins that act in the | The (very) low density |
| immunoglobulin M (IgM) | endoplasmic reticulum | lipoprotein receptor family |
| antibodies | associated degradation | |
| | system (ERAD) | |
| Fc receptors (e.g. human CD16, | Bcl-2 asociated athanogene | UDP-glucose: glycoprotein |
| CD32A, CD32B, CD64) | (Bag-1) | glucosyl transferase (UGGT) |
| multidrug transporter, variously | translocation channel protein | Complement receptor |
| called MultiDrug-Resistance 1 | Sec61p | CD11b/CD18 (Mac-1, CR3) |
| protein (MDR1), P-glycoprotein | | |
| (pleiotropic-glycoprotein), Pgp, | | |
| or P-170 | | |
| casein, αs-casein, β-casein | NFκB | Vitronectin |
| chromozym p450 | c3 | CD79 |
| GrpE | TLR2 | TLR4 |
| TLR9 | (pro)thrombin | Fcε-receptors |
| MAC-2 | | |

^{‡}Monoclonal antibodies developed in collaboration with the ABC-Hybridoma Facility, Utrecht University, Utrecht, The Netherlands.

### Tables 6-11 (Examples 6-20)

TABLE 6
Determination of endotoxin levels in various
protein solutions, using a LAL assay

| | Endotoxin level | Estimated LPS |
|---|---|---|
| Sample^{‡} | (EU) | content (ng/ml) |
| dOVA std (1 mg/ml) | 115.6 | 250 |
| Octagam (lot 5024018434, 50 | 0.033, 0.147 | <0.25 |
| mg/ml) | | |
| Enriched IgIV (HbAGE- | 19.2 | 25 |
| Sepharose) (52 μg/ml in PBS) Lot1 | | |
| Enriched IgIV (HbAGE- | 35 | |
| Sepharose) (103 μg/ml in PBS) | | |
| Lot2, concentrated | | |
| depleted IgIV after contacting | 0.772, 1.112 | 1 |
| HbAGE-Sepharose (27.35 mg/ml) | | |
| HbAGE (1.6 mg/ml) | 0.122 | <0.25 |
| CEALB (Sanquin, lot | 0 | 0 |
| 05C29H120A, 200 mg/ml) | | |
| Mouse 7H2 IgM (in hybridoma | 0 | 0 |
| culture medium) | | |
| Mouse 7H2H2 IgM (purified, in | >3 | |
| PBS) | | |
| Fibronectin finger4-5-FLAG-His | 1.7 | |
| (290 μg/ml in PBS with | | |
| 10% glycerol) | | |
| tPA (Actilyse, 50 μM; 3.65 mg/ml) | 2.7 | |
| dHSA, (20 mg/ml) obtained from | 0.043 | |
| CEALB | | |

^{‡}dOVA was obtained by dissolving ovalbumin to 1 mg/ml in PBS and heating in a PCR thermo-cycler for five cycles from 30° C. to 85° C. at 5° C./minute and quickly back to 30° C., as described above. HbAGE was glycated for 38 weeks and subsequently dialysed against water. dHSA was obtained by denaturing CEALB at 20 mg/ml, at pH 2, at 65° C. for 6 hours, followed by neutralization with NaOH solution to physiological pH.

TABLE 7
Enrichment factors for binding of IgIV after enrichment
on several misfolded crossbeta protein-affinity matrices,
to various misfolded crossbeta proteins^{‡}

| | Enrichment factors obtained with eluted IgIV from | | | |
|---|---|---|---|---|
| | matrices with misfolded crossbeta protein | | | |
| Immobilized | affinity matrix | | | |
| ligand in | | Aβ42/Aβ40 | | |
| binding study | BSA-AGE | fibrils | dHSA | dIgIV |
| BSA-AGE | 30, 15, 44 | 5, 5, 5 | 1.9, 0.7, 5 | 2, 1.8, 2 |
| Aβ42/Aβ40 | 53, 25, 6 | 35, 23, 3 | 18, 11, 3 | 34, 21, 2 |
| fibrils | | | | |
| Aβ42/Aβ40 | 25, 13 | 6, 12 | 7, 0.6 | 5, 9 |
| non-fibrillar | | | | |
| aggregates | | | | |
| nOVA | 1 | 0.6 | 0.8 | 0.5 |
| dOVA | 1.9, 1.3, 2.9 | 0.5, 6, 1.7 | 0.5, 0.25, 1.3 | 1.6, 4, 1.4 |
| dHSA* | 116 | 145 | 186 | 1170 |
| HSA | 0 | 0 | 0 | 0 |
| dmγ-globulins | >1** | 0 | 0 | 0 |
| Mouse γ- | 0 | 0 | 0 | 0 |
| globulins | | | | |

^{‡}enrichment factors are given for each individual experiment. N.d., not determined; 0, no binding; 1, no enrichment
*binding of the Octagam IgIV to HSA and dHSA results in very low signals. Enrichment on a misfolded protein matrix clearly increases binding to dHSA, but determination of accurate enrichment factors is hampered.
**the same is seen for binding of Octagam IgIV to mouse γ globulins. The binding of BSA-AGE enriched IgIV to mγ globulins is increased compared to the starting material. This effect is stronger for denatured γ globulins (Mdγ-globulins)

TABLE 8
Sub-class determination and IgG iso-typing of
preparations of (enriched) affinity regions

| | Octagam IgIV | | | | | Concentrated | |
|---|---|---|---|---|---|---|---|
| | | | % | Enriched IgIV | | enriched IgIV | |
| Ig | mg/ml | % | (datasheet) | mg/ml | % | mg/ml | % |
| IgG | 47.2 | 99.45 | ≧95 | n.d. | — | 0.434 | — |
| IgA | 0.185 | 0.39 | ≦0.4 | n.d. | — | n.d. | — |
| IgM | 0.0756 | 0.16 | ≦0.2 | n.d. | — | n.d. | — |
| IgG1 | 28.6 | 56.1 | 62.6 | 0.0823 | 76.3 | 0.242 | 51.3 |
| IgG2 | 18.6 | 36.5 | 30.1 | n.d. | — | 0.169 | 35.8 |
| IgG3 | 3.20 | 6.3 | 6.1 | 0.0236 | 21.9 | 0.0538 | 11.4 |
| IgG4 | 0.548 | 1.1 | 1.2 | 0.00191 | 1.8 | 0.00675 | 1.4 |

n.d., not detected. For IgG2 the detection limit is <0.093 mg/ml. For IgA the detection limit is 0.0667 mg/ml, for IgM 0.0417 mg/ml, whereas the expected approximate values are one order of magnitude lower.

TABLE 9
Proteins uniquely identified in eluates of matrix with affinity regions with specificity
for misfolded proteins, that was contacted with RA or AL amyloidosis patient samples

| | | # of | |
|---|---|---|---|
| | IPI | peptides in | # of pept [symbol] |
| | accession | sample | in sampl [symbol] |
| Protein name^{‡} | number^{†} | A#¹⁾ | C# |
| Sample A2 and A3 [AL amyloidosis patient plasma] | | | |
| dynein heavy chain domain 3 (gene name: KIAA1503) | IPI00783464 | 1 | 0 |
| IGLC1 protein/immunoglobulin lambda chain | IPI00658130 | 1 | 0 |
| | (also in A4 | | |
| | and A6) | | |
| 25 kDa protein/immunoglobulin lambda locus (gene) | IPI00747752 | 1 | 0 |
| | (also in A4 | | |
| | and A6) | | |
| Hypothetical protein/immunoglobulin lambda variable 4-3 | IPI00382938 | 1 | 0 |
| 25 kDa protein/immunoglobulin lambda chain/rheumatoid | IPI00154742 | 1 | 0 |
| factor G9 light chain (lambda V3)/IGLC1 protein | (also in A4 | | |
| | and A6) | | |
| Sample A4 [RA patient serum] | | | |
| IGLV3-25 protein (immunoglobulin lambda variable 3-25: | IPI00550162 | 1 | 0 |
| synonym: V2-17) | | | |
| Hypothetical protein/immunoglobulin lambda locus/ | IPI00760678 | 1 | 0 |
| immunoglobulin lambda chain C regions (1/2/3)/ | (also in A6) | | |
| immunoglobulin lambda variable V2-14/Ig lambda C3 | | | |
| protein (C2 segment protein/C3 segment protein)/ | | | |
| Hypothetical protein DKFZp667J0810 (Fragment) | | | |
| Hypothetical protein | IPI00784519 | 1 | 0 |
| Hypothetical protein | IPI00784711 | 1 | 0 |
| Hypothetical protein | IPI00784983 | 1 | 0 |
| IGLC2 protein (immunoglobulin lambda C2) | IPI00555945 | 1 | 0 |
| IGLC2 protein (immunoglobulin lambda C2) | IPI00450309 | 1 | 0 |
| Isoform 1 of Centrosomal protein Cep290/Centrosomal | IPI00784201 | 1 | 0 |
| protein Cep290: synonyms (Nephrocystin-6) (Tumor antigen | | | |
| se2-2) | | | |
| Isoform Gamma-B of Fibrinogen gamma chain precursor | IPI00021891 | 1 | 0 |
| IGLC1 protein (immunoglobulin lambda C1)/ | IPI00658130 | 1 | 0 |
| immunoglobulin lambda chain | (also in A2, | | |
| | A3 and A6) | | |
| 25 kDa protein/immunoglobulin lambda chain | IPI00747752 | 1 | 0 |
| | (also in A2, | | |
| | A3 and A6) | | |
| 25 kDa protein/immunoglobulin lambda chain/rheumatoid | IPI00154742 | 1 | 0 |
| factor G9 light chain (lambda V3)/IGLC1 protein | (also in A2, | | |
| | A3 and A6) | | |
| IGLC1 protein (immunoglobulin lambda C1)/ | IPI00719373 | 1 | 0 |
| immunoglobulin lambda chain/immunoglobulin C1 segment | | | |
| protein (fragment) | | | |
| Isoform Long of Antigen KI-67/Antigen KI-67 | IPI00004233 | 1 | 0 |
| Sample A6 [RA patient synovial fluid] | | | |
| IGKC protein (immunoglobulin kappa constant) | IPI00807413 | 10 | 0 |
| Hypothetical protein/immunoglobulin lambda constant 2/ | IPI00760678 | 1 | 0 |
| IGLV2-14 (immunoglobulin variable 2-14/Ig lambda C3 | (also in A4) | | |
| protein (C2 segment protein/C3 segment protein)/IGLC1 | | | |
| (immunoglobulin lambda constant 1)/Hypothetical protein | | | |
| DKFZp667J0810 | | | |
| Hypothetical protein | IPI00807428 | 7 | 1 |
| IGHA1 protein (immunoglobulin heavy constant alpha 1) | IPI00166866 | 10 | 0 |
| Single-chain Fv (Fragment)/Immunoglobulin heavy chain | IPI00748998 | 2 | 0 |
| variable region (fragment)/ | | | |
| Beta-2-glycoprotein 1 precursor/Beta-2-glycoprotein | IPI00298828 | 7 | 0 |
| (Apolipoprotein H) | | | |
| Complement C1q subcomponent subunit C precursor/ | IPI00022394 | 1 | 0 |
| complement component 1, q subcomponent, C chain | | | |
| Complement C1r subcomponent precursor/complement | IPI00296165 | 1 | 0 |
| component 1, r subcomponent/Hypothetical protein | | | |
| DKFZp686O02154 | | | |
| Calmodulin-like protein 5 (Calmodulin-like skin protein) | IPI00021536 | 2 | 0 |
| Complement factor H-related protein 1 precursor/ | IPI00011264 | 2 | 0 |
| Complement factor H-related 1 | | | |
| Isoform DPI of Desmoplakin (250/210 kDa paraneoplastic | IPI00013933 | 1 | 0 |
| pemphigus antigen)/desmoplakin | | | |
| Isoform 1 of Gelsolin precursor/gelsolin | IPI00026314 | 3 | 0 |
| Hypothetical protein/heat shock 70 kDa protein 5 (glucose- | IPI00003362 | 2 | 0 |
| regulated protein = 78 kDa = GRP78 = BiP = HSPA5) | | | |
| IGLC1 protein (immunoglobulin lambda C1)/ | IPI00658130 | 1 | 0 |
| immunoglobulin lambda chain | (also in A2, | | |
| | A3 and A4) | | |
| 25 kDa protein/immunoglobulin lambda locus (gene) | IPI00747752 | 1 | 0 |
| | (also in A2, | | |
| | A3 and A4) | | |
| 25 kDa protein protein/immunoglobulin lambda chain/ | IPI00154742 | 1 | 0 |
| rheumatoid factor G9 light chain (lambda V3)/ | (also in A2, | | |
| immunoglobulin lambda C1 | A3 and A4) | | |
| 26 kDa protein/immunoglobulin kappa variable 1-5 | IPI00738024 | 1 | 0 |

^{‡}Proteins are listed that are identified based on identified peptide masses. For peptide masses that are not unique for a single protein all proteins with the identified sequence are listed
^{†}A# series: analyzed eluate from enriched IgIV-matrix after contacting with indicated patient samples; The control serie C# displayed are the analyses of eluates from control matrix with affinity regions contacted with the same patient samples: background measurement.
¹⁾The IPI accession codes refer to protein entry codes for various protein/peptide databases. When the same protein(s) were also identified in one or more of the other analysed eluates after contacting affinity region-matrix with patient sample, these patient sample codes are given. [symbol]
indicates data missing or illegible when filed

TABLE 10
Effect of enriched affinity regions on immune cells capable of opsonizing proteins

| | DC stimulator | | | | | | |
|---|---|---|---|---|---|---|---|
| | | | | BSA-AGE + | | BSA-AGE + | |
| | PBS | | BSA-AGE + | enr. IgIV + | BSA-AGE + | IVIg + | Poly I-C + |
| marker | Control | BSA-AGE | enr. IgIV | anti-CD32a | IgIV | anti-CD32a | TNFalpha |
| IL-6 (pg/ml) | 104 | 118 | 4433 | 1417 | 191 | 25 | 15119 |
| IL-8 (pg/ml) | 2452 | 1842 | 19316 | 20260 | 4682 | 638 | 26225 |
| cell death (%) | 17 | 11 | 8 | 6 | 14 | 16 | 15 |
| CD80 (MFI ratio) | 8.9 | 4.2 | 11.9 | 11.7 | 5.2 | 3.5 | 25.7 |
| CD83 (% pos. cells) | 5 | 5 | 25 | 23 | 8 | 3 | 66 |
| CD86 (MFI ratio) | 7.3 | 3.8 | 9.5 | 8.6 | 4.3 | 2.9 | 14.4 |
| CD40 (MFI ratio) | 16.1 | 9.5 | 13.4 | 10.6 | 9.6 | 5.8 | 20.7 |

TABLE 11
Anti-CCP titers in various preparations of human affinity regions

| | [anti-CCP titer] | |
|---|---|---|
| Ig preparation | (U/ml) | Enrichment fact [symbol] |
| 1. Octagam IgIV, 50 mg/ml | 3 | 1 |
| 2. γ-globulins, 10 mg/ml | 2.6 | — |
| 3. Gammagard IgIV, 52 mg/ml | 3.1 | — |
| 4. enriched IgIV, 0.1 mg/ml | 2.7 | 437 |

When the anti-CCP titer in serum of an individual is >10 U/ml, the serum is designated as anti-CCP antibody positive. T [symbol] measured titers of 2.6-3.1 U/ml are well within the detection limits of the EliA system, and this range of titers is regularl [symbol] measured for sera of healthy individuals.
‡The enrichment for the concentration of anti-CCP antibodies is determined with enriched IgIV, in comparison with Oct [symbol] IgIV from which enriched IgIV was selected using HbAGE-Sepharose affinity matrix. [symbol]
indicates data missing or illegible when filed

### BRIEF DESCRIPTION OF THE DRAWINGS

FIG. 1. Human IgIV binds specifically to misfolded glycated proteins.

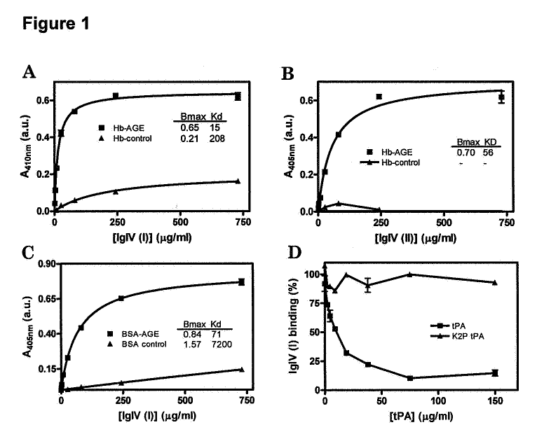

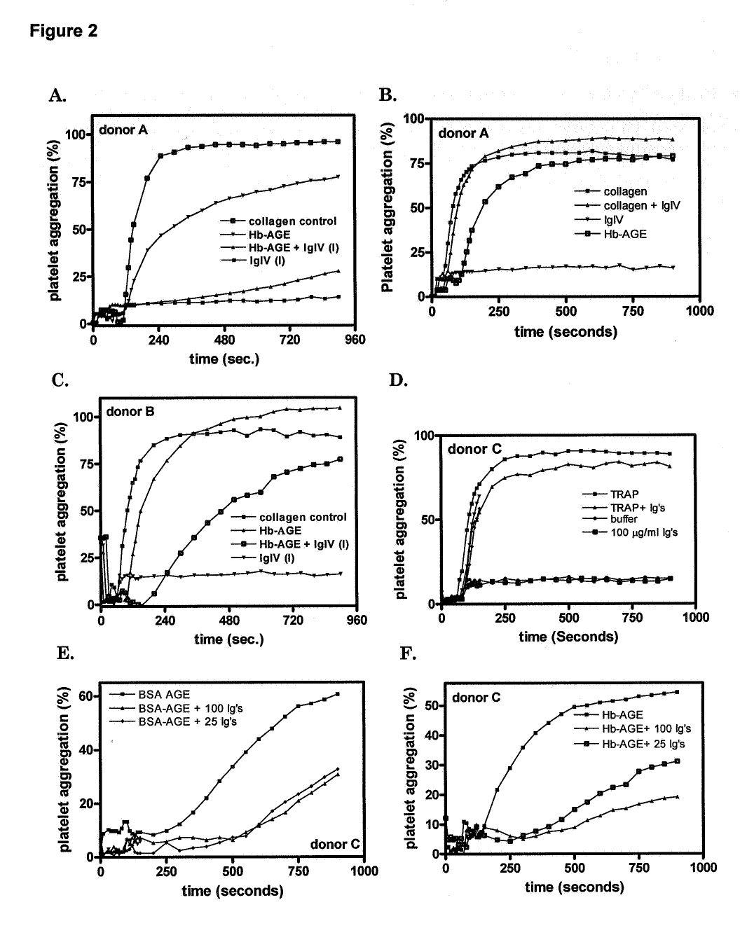

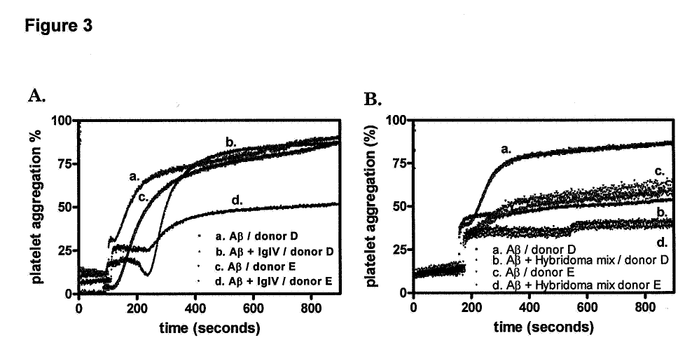

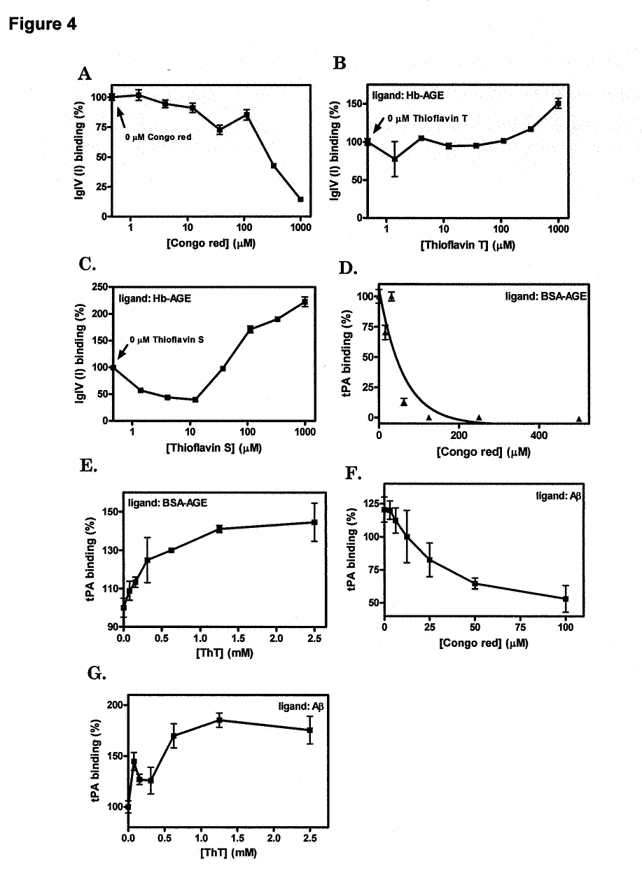

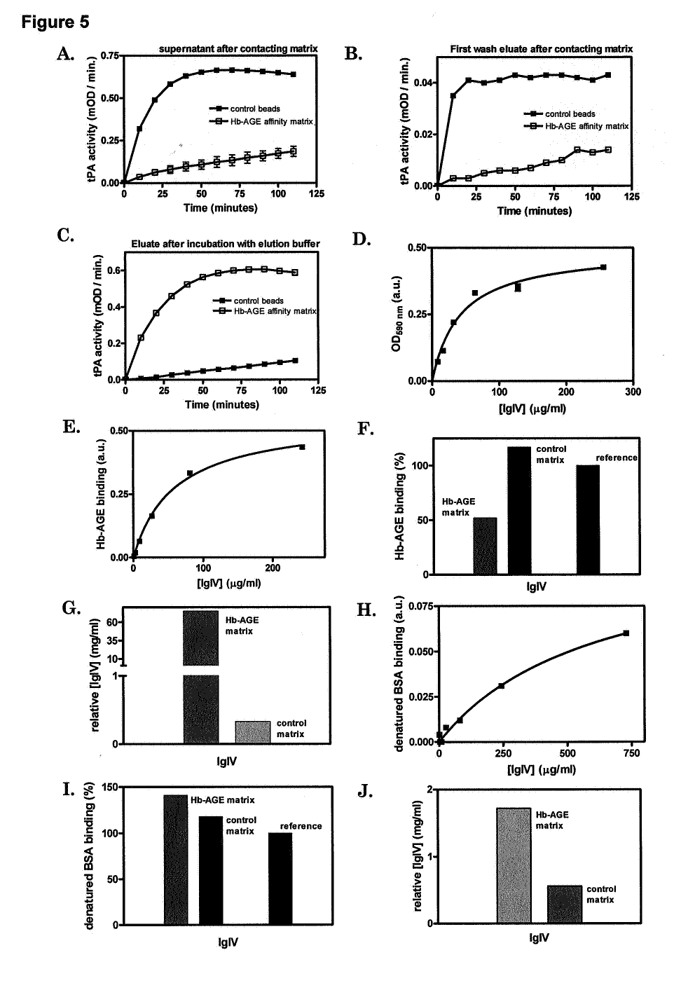

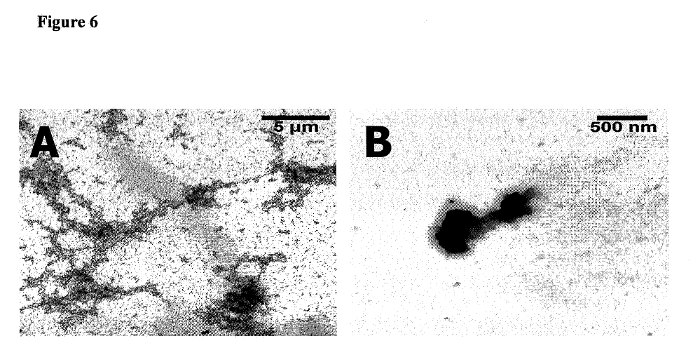

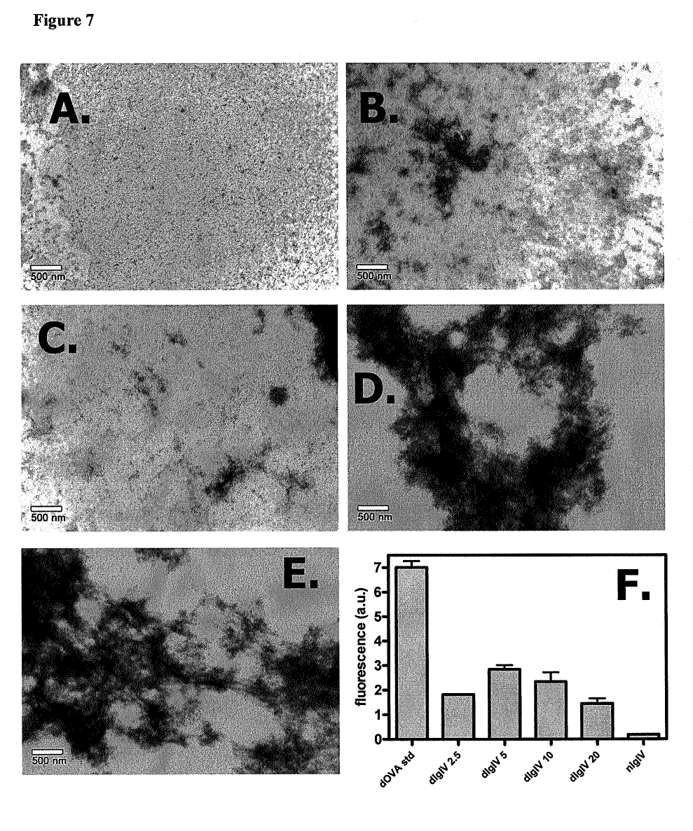

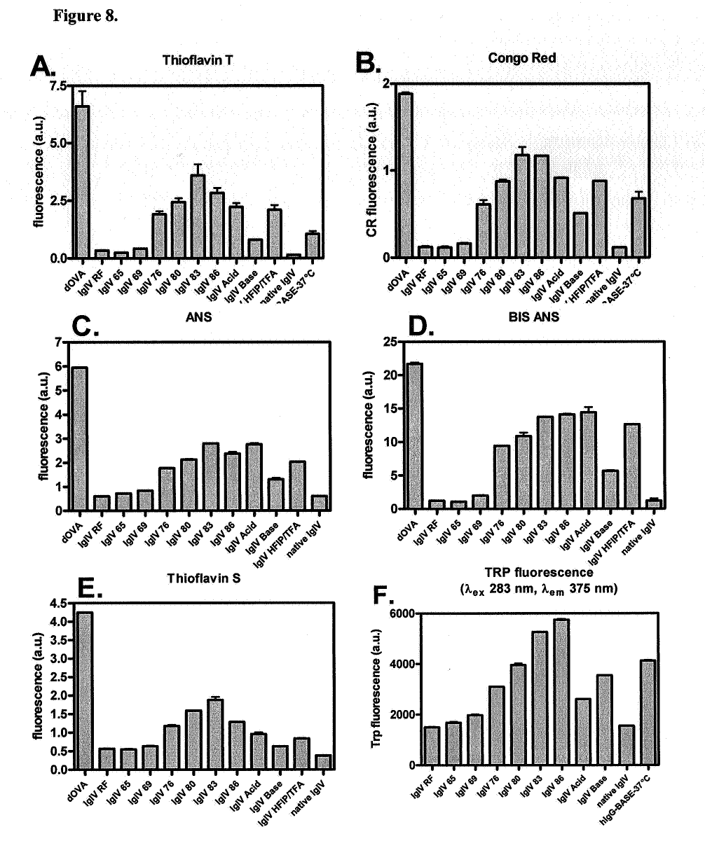

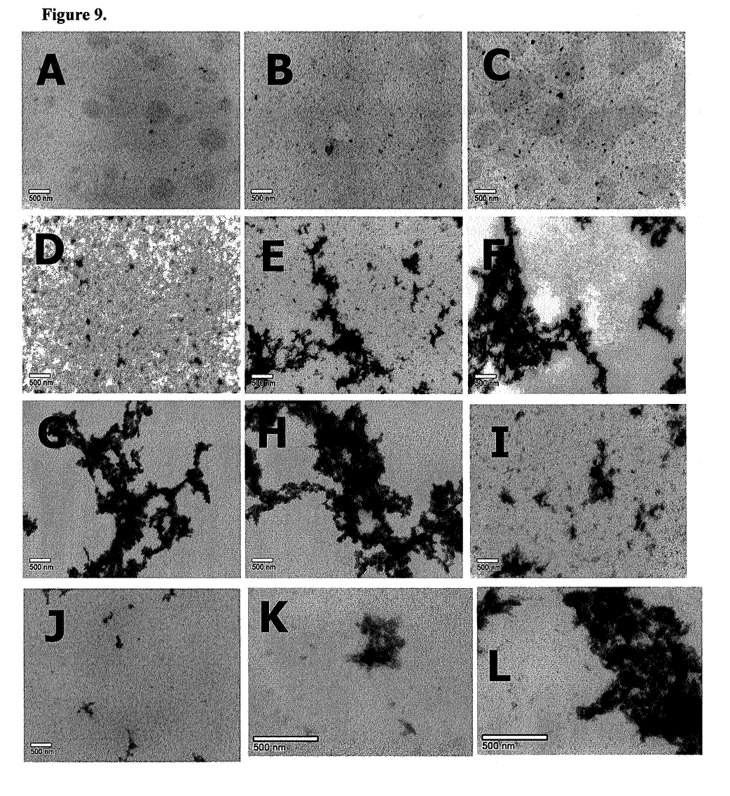

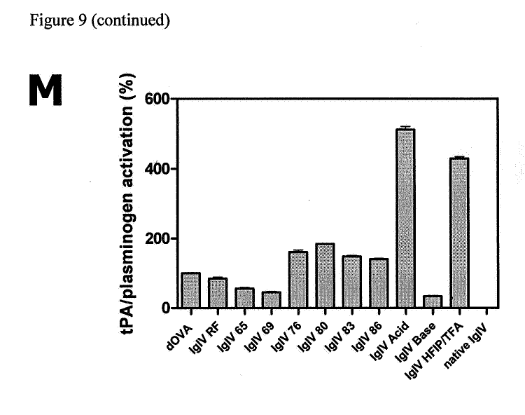

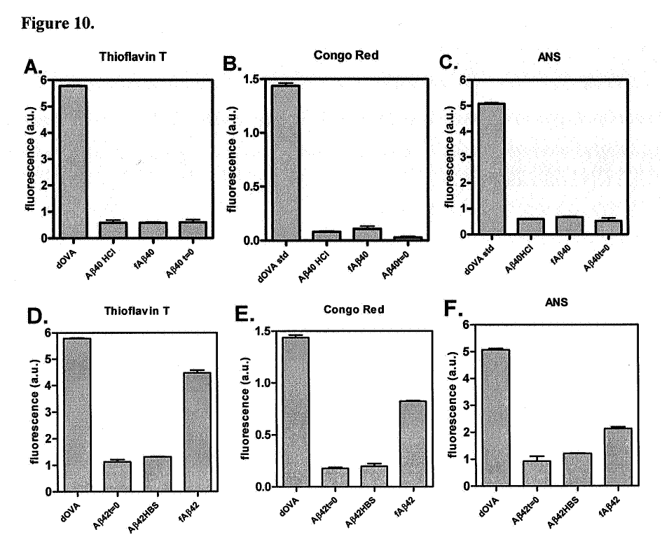

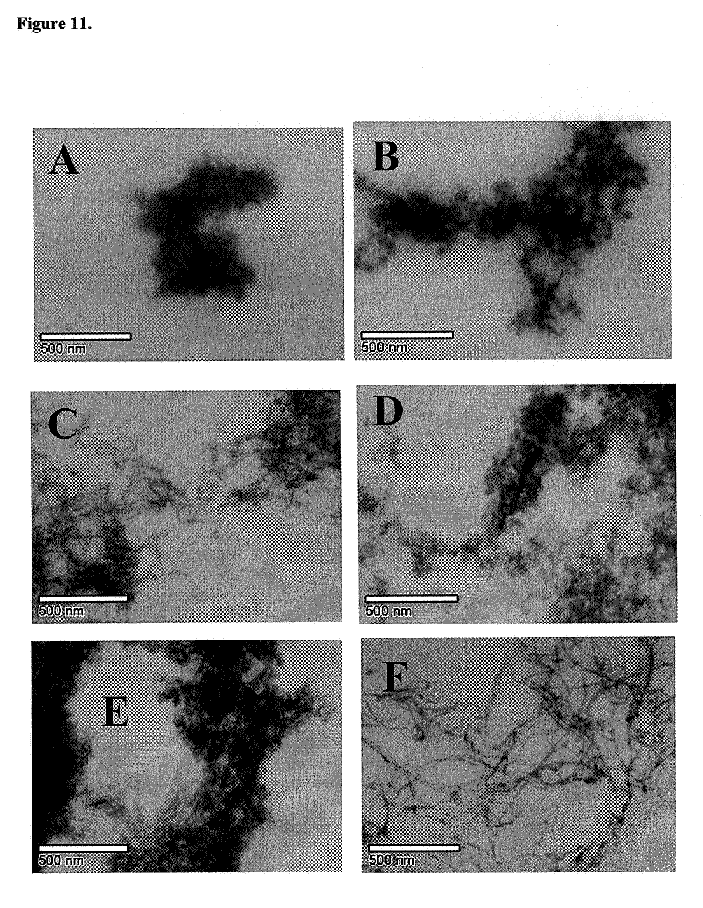

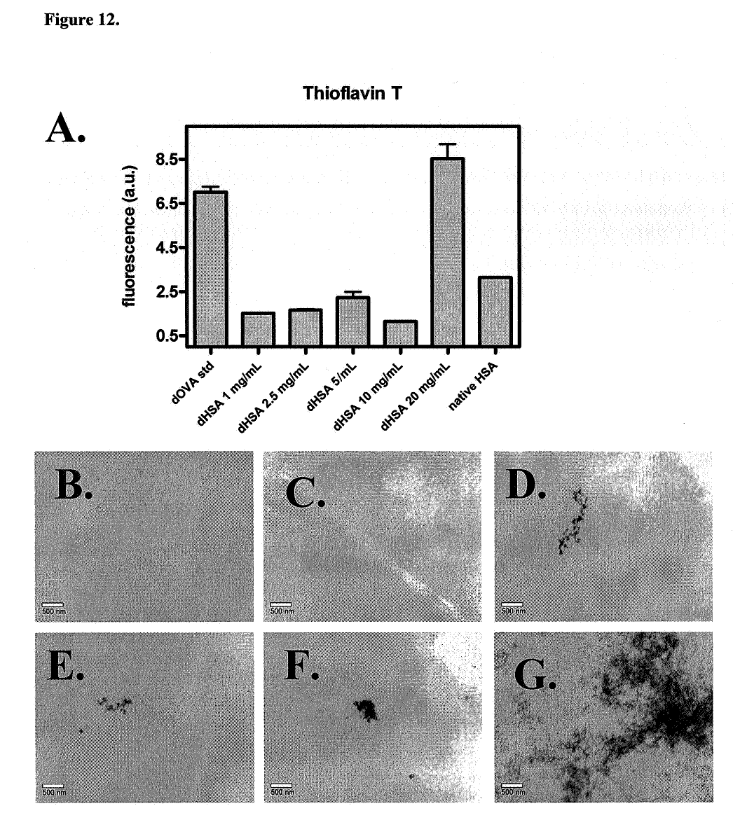

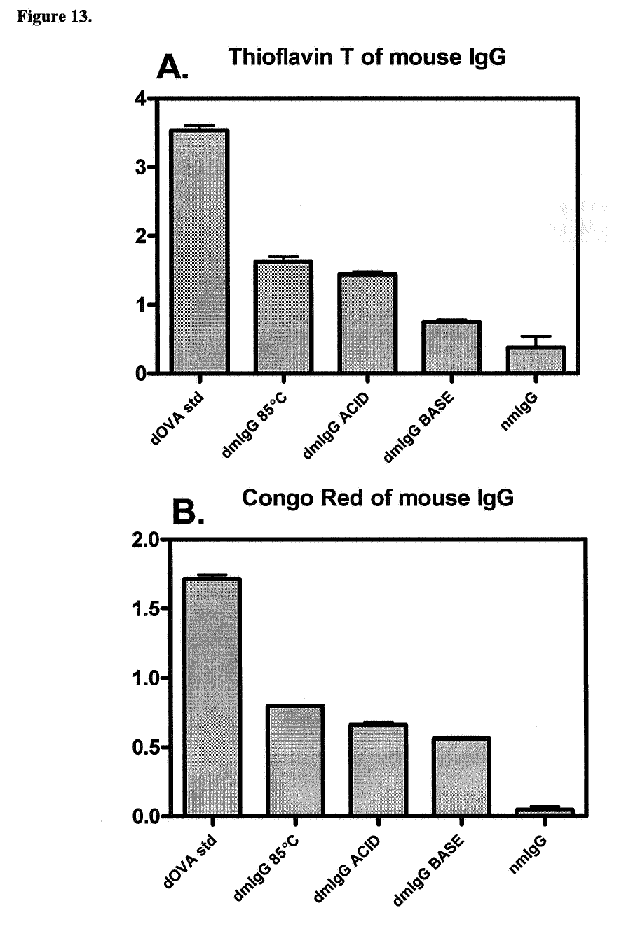

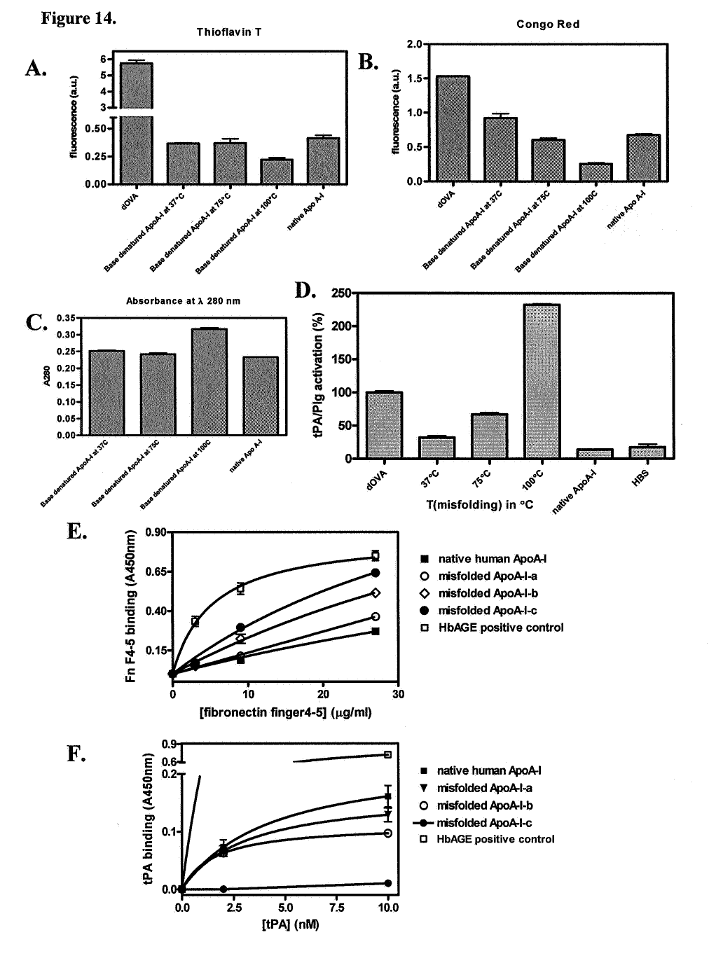

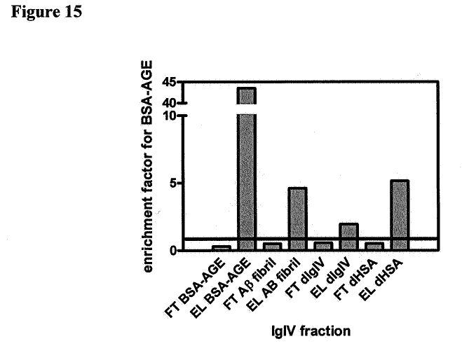

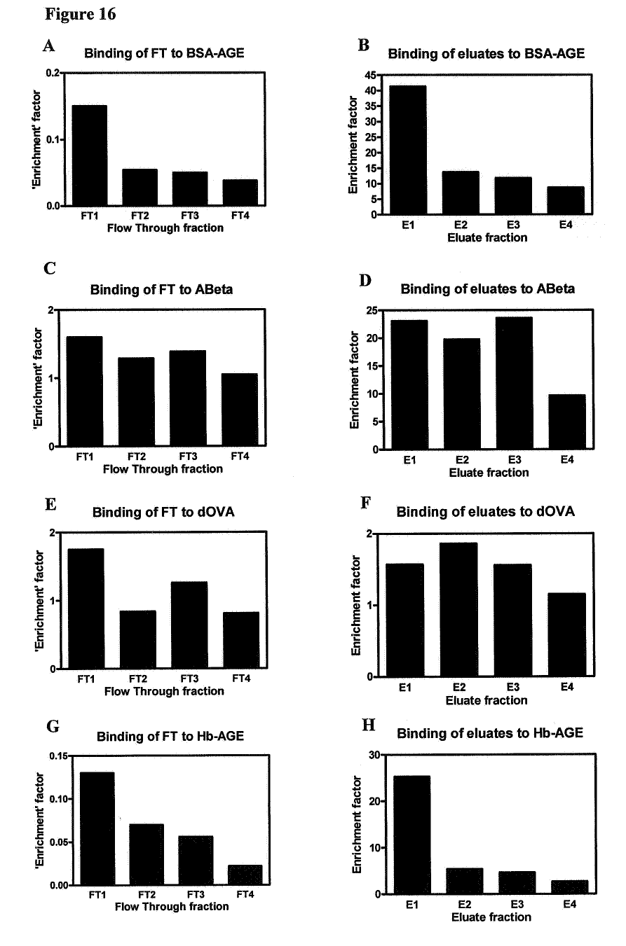

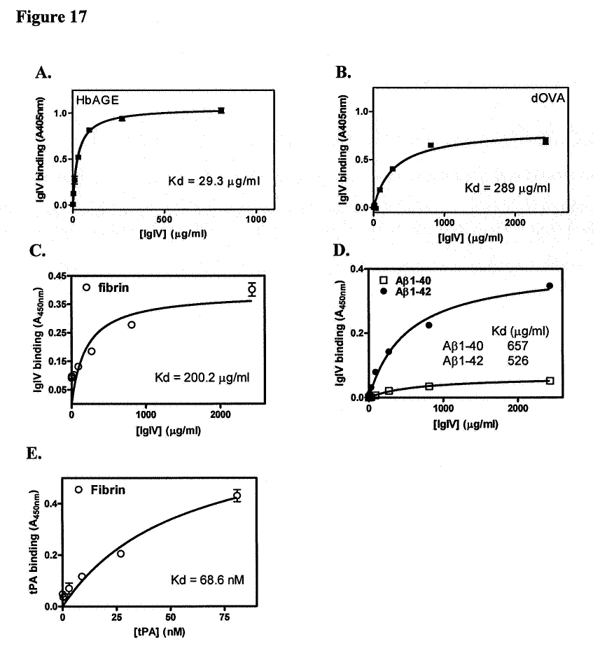

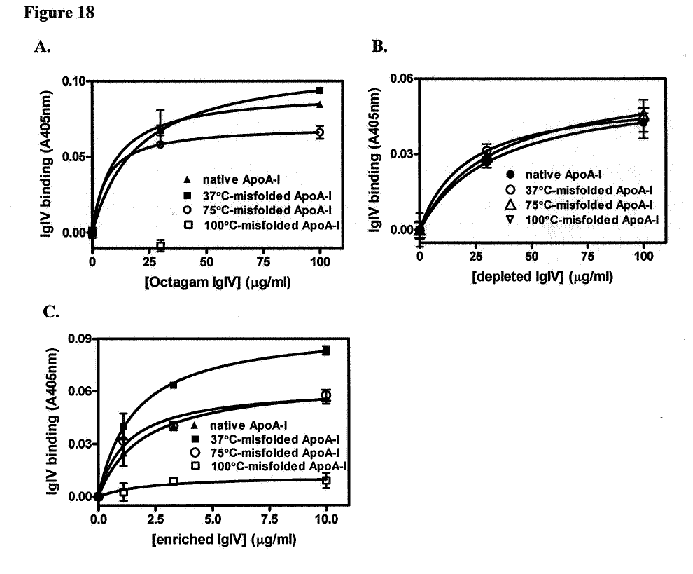

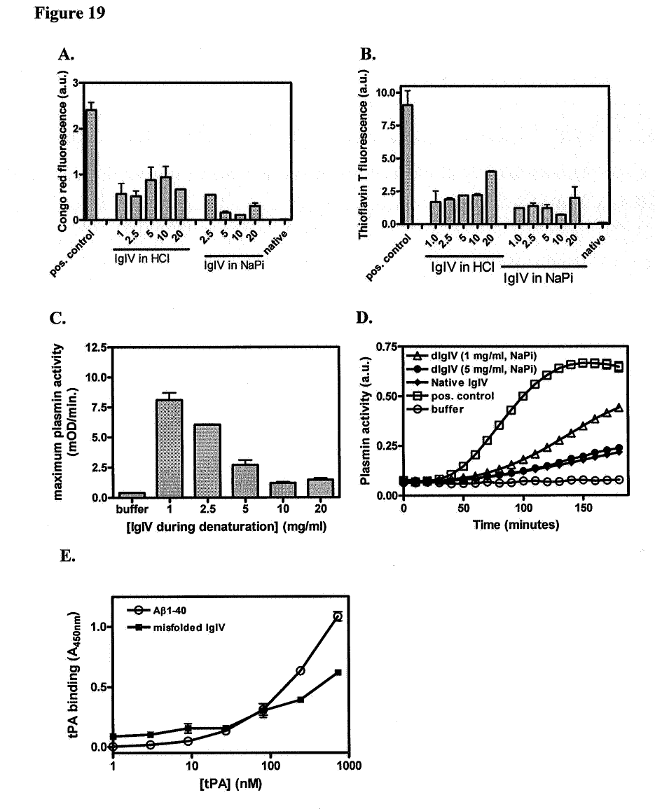

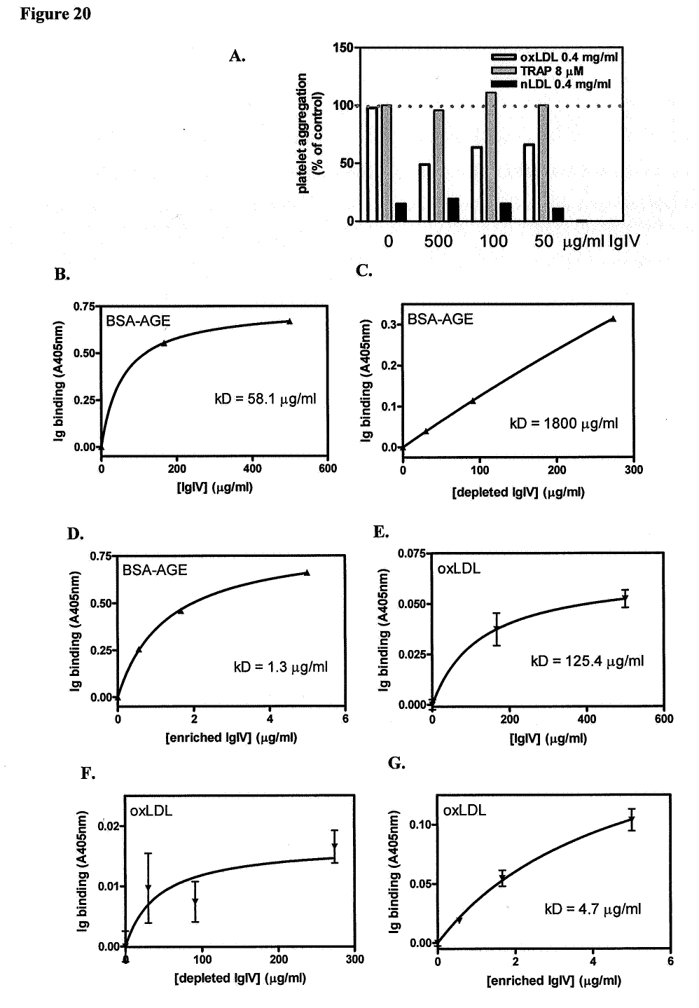

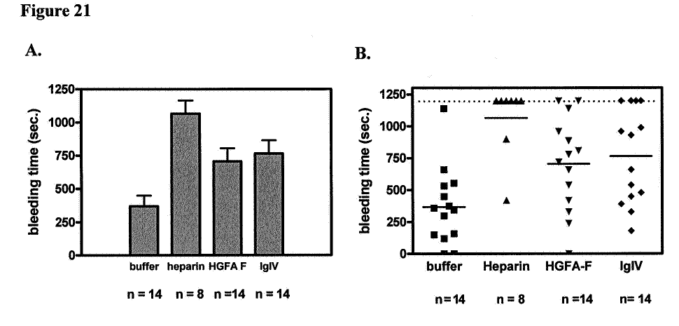

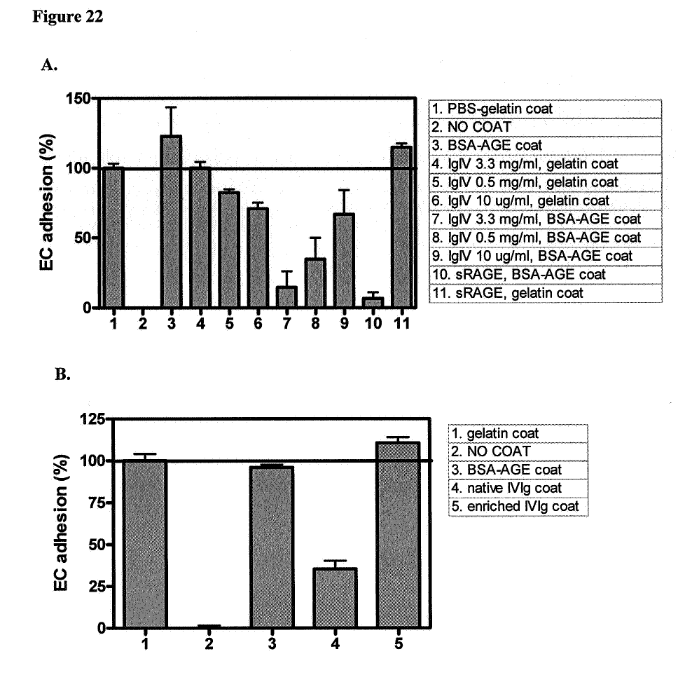

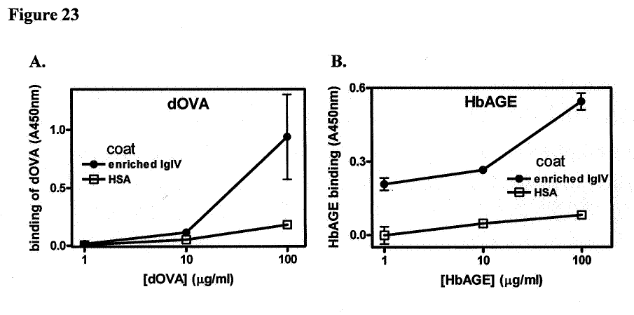

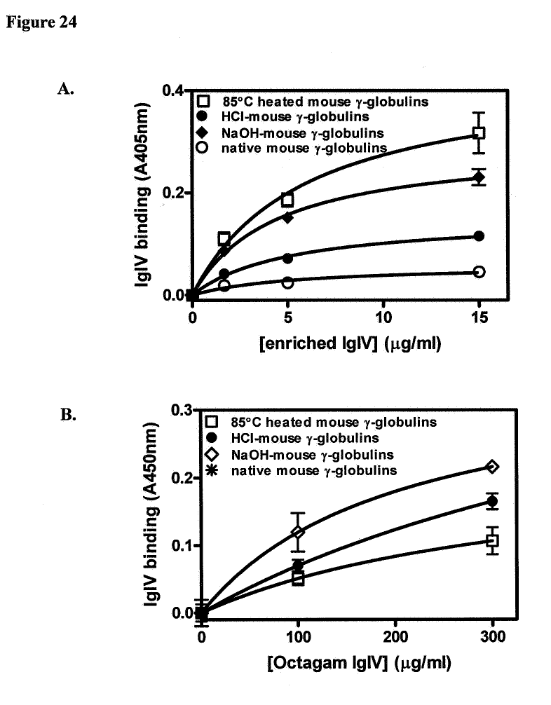

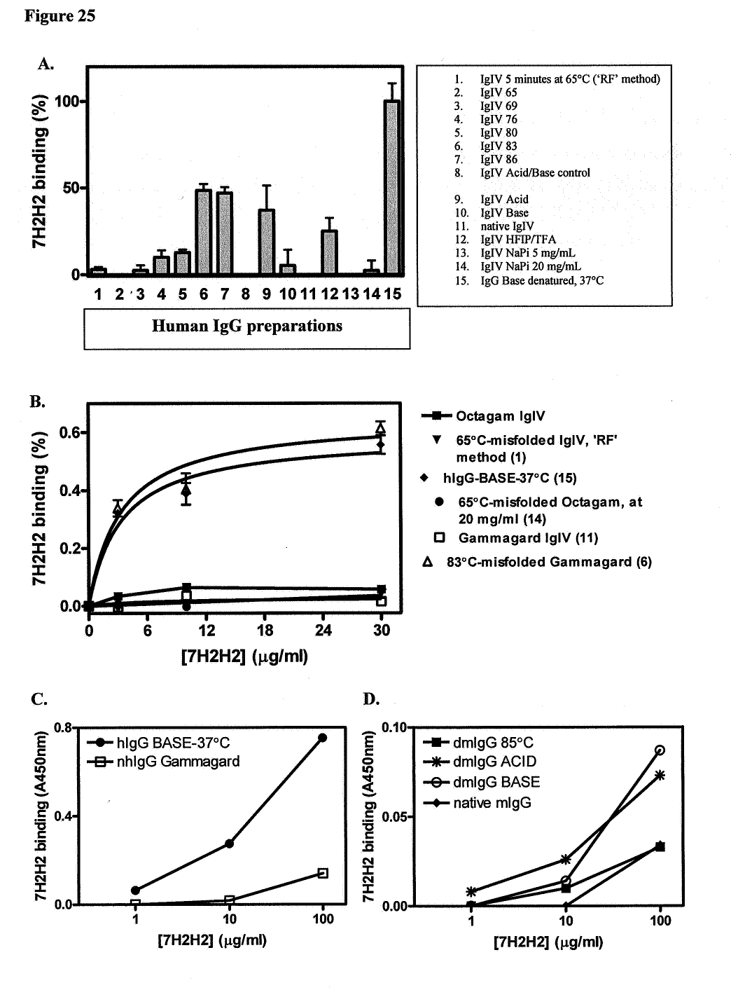

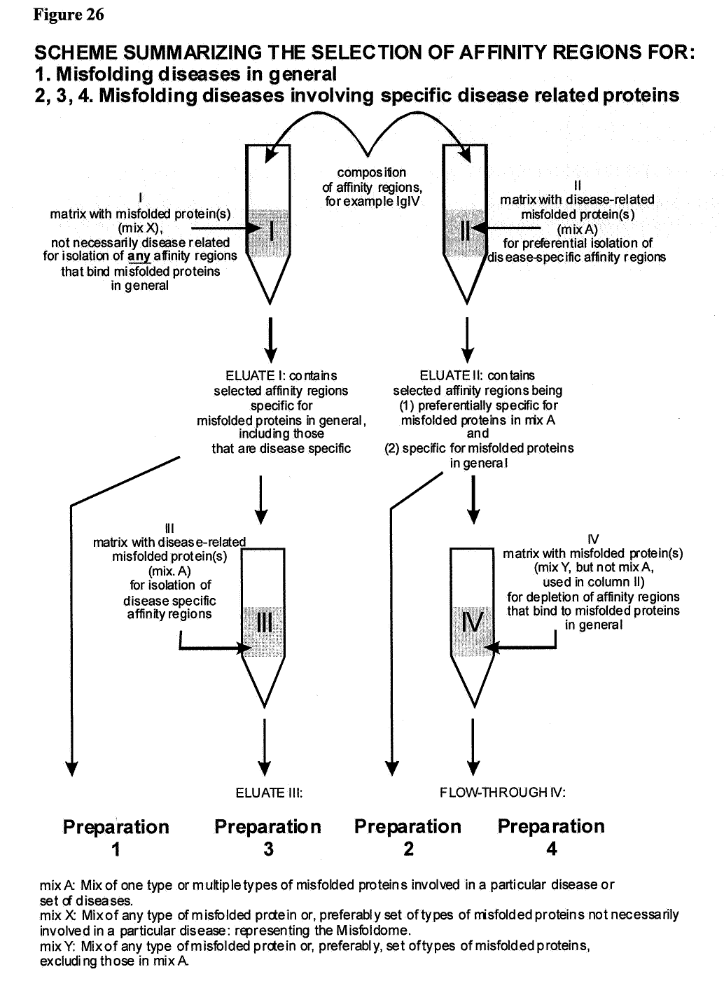

In ELISA set-ups the binding of human IgIV for therapeutical usage, obtained from two manufacturers, I and II, was assessed with immobilized glycated proteins. A. Binding of IgIV from manufacturer I (IgIV (I)) to coated glycated human haemoglobin (Hb-AGE), freshly dissolved Hb and aggregated amyloid-β peptide (Aβ) was tested. B. Binding of IgIV from manufacturer II (IgIV (II)) to coated Hb-AGE, freshly dissolved Hb and aggregated Aβ was tested. C. Binding of IgIV (I) to coated glyeated albumin (BSA-AGE), freshly dissolved control albumin and FP13 K157G amyloid was analyzed. D. The influence of tPA and K2P tPA on the binding of 15 μg/ml IgIV (I) to coated Hb-AGE was addressed by adding concentration series of tPA or K2P tPA to the IgIV (I) incubation mixture. Ten mM of εACA was added to the mixture to avoid binding of tPA to exposed lysine or arginine side chains.

FIG. 2: Platelet aggregation induced by misfolded glycated proteins with amyloid-like conformation is inhibited by IgIV and a mixture of monoclonal antibodies.

Platelet aggregation after introduction of collagen or TRAP (positive controls), buffer (negative control) or misfolded amyloid-like glycated albumin or haemoglobin was followed in an aggregometer using isolated platelets from freshly drawn citrated plasma in HEPES-Tyrode buffer. The proposed inhibitory properties of human IgIV and murine monoclonal antibodies raised against four different amyloid structures, on platelet aggregation was assessed. A. IgIV purchased from manufacturer I effectively inhibits the glycated haemoglobin-induced aggregation of platelets of human donor ‘A’. IgIV (I) itself has no effect on platelets, that is to say, no aggregation is induced by adding IgIV (I) to platelets. IgIV (I) concentration used was 4.7 mg/ml, the Hb-AGE concentration was 18 μg/ml, collagen was used at a concentration of 10 μg/ml. B. Influence of 10 μg/ml collagen, 18 μg/ml Hβ-AGE, 4.7 mg/ml IgIV (I) and 18 μg/ml Hb-AGE that was preincubated with 4.7 mg/ml IgIV (I) on aggregation of platelets of donor A was determined. C. Similar to the experiment performed with platelets of donor A (A.), platelet aggregation with platelets of donor ‘B’ was followed in time. Now, 10 μg/ml collagen, 90 μg/ml Hb-AGE, 4.7 mg/ml IgIV (I) and 90 μg/ml Hb-AGE preincubated with 4.7 mg/ml IgIV (I) was used. D. In a control experiment with platelets of donor ‘C’ 5 μM TRAP was used as a positive control. The influence of 100 μg/ml of a mixture of five monoclonal antibodies with affinity for misfolded proteins with crossbeta structure conformation was determined with TRAP as activator of aggregation, or with HEPES-Tyrode buffer control. E., F. Platelet aggregation (donor C) was induced by 25 μg/ml glycated bovine serum albumin (BSA-AGE, E.) or glycated human haemoglobin (Hb-AGE, F.). Inhibition of this aggregation by 25 or 100 μg/ml mixture of five monoclonal antibodies with affinity for misfolded proteins with crossbeta structure conformation was determined.

FIG. 3. Blood platelet aggregation is induced by amyloid-β, and inhibited by IgIV or monoclonal antibodies.

A., B. Induction of platelet aggregation by 50 μg/ml amyloid-β is inhibited when 2.5 mg/ml IgIV (I) is pre-incubated with amyloid-β (A.), or when 160 μg/ml mixture of five monoclonal antibodies that bind to misfolded proteins (B.) is preincubated with amyloid-β. Platelets of two donors D and E are analyzed separately.

FIG. 4. Amyloid-specific small compounds influence binding of IgIV or tPA to immobilized misfolded proteins differently.

In ELISA set-ups binding of IgIV or tPA, a multiligand binding protein with affinity for misfolded proteins that comprise the crossbeta structure tertiary/quarternary fold, was analyzed under influence of concentrations series of amyloid-specific dyes Congo red and Thioflavin T. A-C. The influence of amyloid-specific dyes Congo red (A.), Thioflavin T (B.) and Thioflavin S(C.) on binding of 15 μg/ml IgIV (I) to immobilized Hb-AGE was addressed by preincubating the IgIV (I) with concentration series of the three dyes before adding the solutions to ELISA plates. D., F. Influence of Congo red on binding of a suboptimal concentration of tPA to coated BSA-AGE (D.) or Aβ (F.). E., G. Influence of Thioflavin T on binding of a suboptimal concentration of tPA to coated BSA-AGE (E.) or Aβ (G.).

FIG. 5. An affinity matrix with the ability to bind proteins that bind to misfolded proteins with crossbeta structure conformation.

Glycated and misfolded human haemoglobin was linked to CNBr-Sepharose and the ability to bind proteins with affinity for misfolded proteins that comprise the crossbeta structure fold was determined by analyzing tPA binding. Next, the affinity matrix was applied to isolate a subset of immunoglobulin molecules from IgIV-I comprising affinity regions for cross-β structure and/or proteins comprising cross-β structure. A. Hb-AGE Sepharose and empty control beads were incubated with 6 μM tPA solution and the supernatant was subsequently analyzed for the presence of tPA activity by adding tPA chromogenic substrate S2765. B. After incubation with tPA the Hb-AGE Sepharose and the control beads were washed several times with wash buffer. The presence of tPA in the first wash eluate was again analyzed by following tPA substrate S2765 conversion at 37° C. in time. C. After extensive washing bound tPA was eluted from empty control Sepharose beads and Hb-AGE Sepharose with high salt. Ten times diluted eluate was analyzed for the presence of tPA by adding S2765. D. Standard curve of the absorbance at 590 nm of diluted IgIV stock (Octagam), stained with ADV01. E. Standard curve of binding of a dilution series of IgIV stock (Octagam) to Hβ-AGE, as determined with ELISA. F. Binding to immobilized Hb-AGE of 1000 times diluted IgIV stock, IgIV after contacting with Hb-AGE-Sepharose and IgIV after contacting with control matrix, as assessed with an ELISA. G. Binding to immobilized Hb-AGE of IgIV eluted from Hb-AGE-Sepharose and IgIV eluted from control matrix, as assessed with an ELISA. Signals are given as relative numbers, as calculated from the IgIV stock binding curve (See Figure E). H. Standard curve of binding of a dilution series of IgIV stock (Octagam) to heat-denatured BSA, as determined with ELISA. I. Binding to immobilized heat-denatured BSA of 1000 times diluted IgIV stock, IgIV after contacting with Hb-AGE-Sepharose and IgIV after contacting with control matrix, as assessed with an ELISA. J. Binding to immobilized heat-denatured BSA of IgIV eluted from Hb-AGE-Sepharose and IgIV eluted from control matrix, as assessed with an ELISA. Signals are given as relative numbers, as calculated from the IgIV stock binding curve (See Figure H).

FIG. 6. TEM analysis of misfolded, through glycation, albumin (BSA-AGE) and hemoglobin (HbAGE)

The images show that BSA-AGE (A.) and HbAGE (B.) form non-fibrillar amorphous aggregates.

FIG. 7. Misfolding of Octagam IgIV induces crossbeta structure

A-E. TEM analysis of misfolded IgIV Octagram at 1 mg/ml (A), 2.5 mg/ml (B), 5 mg/ml (C), 10 mg/ml (D) and 20 mg/ml (E) in 10 mM NaPi buffer pH 8.1. F. Thioflavin T analysis of misfolded Octagam IgIV. It is seen that different conditions of denaturation result in misfolded proteins with different TEM and Thioflavin T characteristics.

FIG. 8. Misfolding of Gammagard IgIV induces crossbeta structure

Thioflavin T (A), Congo red (B), ANS(C), Bis-ANS (D) and Thioflavin S (E) fluorescence of various misfolded IgIV Gammagard preparations. F. Tryptophan fluorescence of the various misfolded IgIV Gammagard preparations when the fluorescence intensity at 375 nm is measured upon exciting at 283 nm.

FIG. 9. Misfolding of Gammagard IgIV induces aggregation, accompanied with ability to activate tPA/plasminogen.

TEM analysis of A. native IgIV Gammagard, and various forms of misfolded IgIV Gammagard, i.e. B. IgIV RF, C. IgIV 65, D. IgIV 69, E. IgIV 76, F. IgIV 80, G. IgIV 83 Gammagard, H. IgIV 86, I. IgIV Acid and J. IgIV Base. K. IgIV HFIP/TFA, L. hIgG-BASE-37° C., M. tPA mediated plasmin generation upon exposure to various denatured IgIV Gammagard preparations at a final concentration of 100 μg/ml. Co-factor stimulation of dOVA at 40 μg/ml was set arbitrarily to 100%.

FIG. 10. ThT, Congo red and ANS analysis of Aβ preparations.

FIG. 11. TEM analysis of Aβ.

A. Aβ40t=0, B. Aβ40HCl, C. fAβ40 (i.e. stored for 168 h), D. Aβ42t=0, E. Aβ42HBS, and F. fAβ42 (i.e. HCl treatment at 37° C. for 24 h).

FIG. 12. Analysis of HSA structure.

Thioflavin T fluorescence of native and denatured HSA (A) and TEM analysis of native HSA (B) and HSA denatured at 1 mg/ml (C), 2.5 mg/ml (D), 5 mg/ml (E) 10 mg/ml (F) or 20 mg/ml (G).

FIG. 13. Enhanced fluorescence of ThT and CR with misfolded mouse IgG.

Thioflavin T (A) and Congo red (B) fluorescence of heat denatured mouse IgG (dmIgG 85° C.), acid denatured mouse IgG (dmIgG ACID), base denatured mouse IgG (dmIgG BASE) and native mouse IgG (nmIgG). The mouse IgG preparation used is a composition of mouse γ-globulins.

FIG. 14. Structure analysis of human ApoA-I.

(A) ThT fluorescence, (B) Congo red fluorescence, (C) A280 nm protein determination, (D) tPA/plasminogen (Plg) activation assay and (E) binding of fibronectin F4-5-FLAG-His to immobilized ApoA-I and HbAGE (positive control). Background signals obtained with control buffer coated wells are subtracted from signals obtained with corresponding Fn F4-5 dilution series on immobilized proteins. Misfolded ApoA-I a to c: a=incubated for 30 minutes at 37° C. after adding NaOH to a final concentration of 100 mM to native ApoA-I stock; addition of HCl to a final concentration of 100 mM after warming; b=as in a, now heated to 75° C.; c=as in a, b, now heated to 100° C. (F). tPA binding to the ApoA-I preparations and HbAGE, similar as in A. For clearity, a two-segment y-axis is displayed, because absolute signals obtained with tPA and ApoA-I preparations are substantially lower than the signal obtained with HbAGE.

FIG. 15. Enhanced binding to misfolded BSA-AGE of affinity regions that are enriched using indicated misfolded crossbeta proteins coupled to matrices.

In the figure are the misfolded proteins indicated that were immobilized on a matrix. ‘FT’, affinity matrix flow-through; ‘EL’, affinity matrix eluate, or recovered fraction after elution of affinity regions bound to the indicated misfolded proteins. The solid line at an enrichment factor of 1 indicates the border between depletion or enrichment with respect to binding of affinity regions to, like in this illustrative example, BSA-AGE.

FIG. 16. Binding of enriched and depleted IgIV to misfolded crossbeta proteins after contacting IgIV with misfolded crossbeta BSA-AGE affinity matrix.

Octagam IgIV was incubated with BSA-AGE Sepharose. One part of the flow trough (FT) fractions was tested in an ELISA for binding to BSA-AGE, the remaining FT was applied again to a fresh amount of BSA-AGE matrix (A). The eluate fractions E were collected and tested in an ELISA for binding to BSA-AGE as well (B). The enrichment factor is given as the binding to misfolded protein per mass unit, compared to Octagam IgIV starting material. During the successive binding steps more BSA-AGE binding Ig molecules are isolated from the Octagam pool resulting in a decreasing enrichment factor for the successive FT fractions. Ig molecules bound specifically to the BSA-AGE matrix are eluted from the affinity matrix (eluates, E). Enrichment factors of FTs and Eluate fractions were also determined with Aβ (C and D), dOVA (E and F) and HbAGE (G and H).

FIG. 17. Binding of Octagam IgIV to various proteins with crossbeta conformation, including fibrin, analysed with ELISAs.

A-D. ELISAs showing binding of Octagam IgIV to immobilized Hb-AGE (A., positive control), dOVA (B.), fibrin (C.), and Aβ 1-40 and Aβ 1-42 (D.). E. Binding of tPA to fibrin (positive control for C.).

FIG. 18. Binding of various IgIV preparations to various misfolded human plasma apolipoprotein A-I preparations.

A. In an ELISA binding of Octagam IgIV to immobilized native ApoA-I and ApoA-I misfolded by adding NaOH to a final concentration of 100 mM, followed by a 30-minutes incubation at 37° C., or 75° C., or 100° C., was assessed. No binding is seen with the ApoA-I that was heated at 100° C. B. ELISA as in A., with depleted IgIV flow-through that was recovered from an HbAGE-affinity matrix after contacting the matrix with Octagam IgIV. Again, no binding is seen with ApoA-I heated to 100° C. C. ELISA as in A. and B., with the enriched IgIV eluate after contacting HbAGE-affinity matrix with Octagam IgIV.

FIG. 19. Congo red and Thioflavin T fluorescence enhancement, tPA binding and tPA/plasminogen activation by misfolded IgIV.

IgIV was heat-denatured at increasing concentrations, either at 65° C. in NaPi buffer pH 8.1, or in HCl, pH 2 for 6 hours at 65° C. Congo red (A) and Thioflavin T fluorescence enhancement (B) was measured. Congo red fluorescence was not tested with IgIV denatured at 1 mg/ml. Activation of tPA/plasminogen by native IgIV and heat-denatured misfolded IgIV, heated at 1 mg/ml or 5 mg/ml is determined using a chromogenic substrate for plasmin. C. Maximum plasmin activity was determined with heated IgIV that was misfolded at the indicated concentrations. D. Representative graph showing plasmin activity induced by IgIV misfolded in NaPi buffer at 1 mg/ml and 5 mg/ml. E. Binding of tPA to Aβ40t=0 and misfolded IgIV.

FIG. 20. Aggregation of human blood platelets by oxLDL is inhibited by IgIV; affinity of enriched IgIV for oxLDL, as compared with non-enriched starting material and depleted IgIV, collected as flow-through, after exposure of IgIV to misfolded HbAGE-affinity matrix.

A. Influence of IgIV on oxLDL-induced platelet aggregation. Aggregation induced by TRAP is maximal and is arbitrarily set to 100%. The influence of a concentration series of IgIV is assessed by pre-incubating the native LDL control or oxLDL with IgIV, before addition to the platelet suspension and start of the aggregation experiment. B.-D. ELISA: Binding to immobilized BSA-AGE of Octagam IgIV (B.), IgIV depleted from affinity regions which were immobilized on a HbAGE-matrix (C.), and IgIV that was enriched by applying an HbAGE-affinity matrix (D.). E.-G. display binding to oxLDL of the same three indicated affinity region stocks. E. starting material, Octagam IgIV, F. IgIV depleted from affinity regions with affinity for crossbeta proteins and/or crossbeta induced conformation in proteins, and G. binding of enriched IgIV to oxLDL. If possible, kD values are calculated to obtain a comparable quality measure for the experiments. The ratio between the kD's obtained for binding of IgIV to oxLDL and for binding of enriched IgIV, using a misfolded HbAGE matrix, to oxLDL is 27, showing that the enrichment factor obtained with the followed procedure is 27 for binding of affinity regions to misfolded ApoB100.

FIG. 21. Influence of crossbeta structure binding compounds IgIV and HGFA F on bleeding time in an in vivo mouse bleeding time assay.

A. In a mouse tail cut assay, both HGFA F (approximately 234 μg/ml final concentration) and IgIV (approximately 2.5 mg/ml final concentration) prolong bleeding time significantly. Buffer (PBS) was used as a reference for bleeding time. Ten IE heparin per mouse was used in a positive control group of prolonged bleeding time. Calculated mean bleeding times and error bars are given. B. The averaged data as shown in A. are now displayed in a scatter plot in order to provide insight in the distribution of measured bleeding times. Note: bleeding times exceeding 20 minutes were set to 20 minutes and bleeding was actively stopped, and in addition, excessive bleeding resulting in blood loss of over 200 μl was also set to a bleeding time of 20 minutes and bleeding was actively stopped (both procedures are according to the protocol that was approved by the local ethical committee).

FIG. 22. Adhesion of cells to misfolded proteins and modulation with enriched affinity regions.

A. ECs bind to wells of a culture plate that are pre-coated with gelatin (arbitrarily set to 100%) or BSA-AGE. When Octagam IgIV is titrated in the cell suspension, adherence to glycated albumin is dose dependently inhibited. Similar inhibition of adherence is seen with recombinant soluble fragment of human RAGE. B. ECs bind preferentially to enriched IgIV over native IgIV coated at the same concentration. Positive controls for adherence are gelatin (binding set to 100%) and BSA-AGE. Negative control is adherence of cells to cell culture plate wells that were not coated with protein at all (0% adherence).

FIG. 23. Depletion of solutions from misfolded proteins using enriched IgIV

A-B. Extraction of misfolded dOVA (A.) or HbAGE (B.) from a protein solution by using enriched IgIV affinity regions that are immobilized on a solid support, i.e. the wells of an ELISA plate. Negative control: HSA immobilized on the solid support.

FIG. 24. Binding of enriched human IgIV and Octagam IgIV to various forms of misfolded mouse IgG.

A. Binding of enriched human IgIV to misfolded mouse IgG was assessed in a direct ELISA with immobilized mouse IgG preparations. B. In a second approach, first anti-mouse IgG antibody was coated onto the wells of a 96-wells plate, followed by binding of various mouse IgG preparations, and overlays with a concentration series of Octagam human IgIV.

FIG. 25. Binding of Mouse hybridoma IgM 7H2H2 to various forms of misfolded human γ-immunoglobulins and mouse self-γ-globulins.

Binding of mouse hybridoma IgM 7H2H2 to various forms of misfolded human IgG preparations was assessed in ELISAs. A. 7H2H2 IgM at 12.5 μg/ml in PBS/0.1% Tween 20 was tested for binding to 15 different human IgG preparations, as indicated in the ‘General Materials and Methods for Example 6-20’ section. B. In a second experiment, purified hybridoma clone 7H2H2 IgM at the indicated concentrations was again analyzed for binding to five human IgG preparations. Control native IgG's are Gammagard IgIV and Octagam IgIV. Numbers for the IgIV preparations refer to IgIV preparations used in A. (See also the text). C. Binding of mouse hybridoma IgM 7H2H2 to hIgG-BASE-37° C. and native IgIV Gammagard. D. Binding of 7H2H2 to various preparations of misfolded mouse IgG and native mouse γ-globulins.

FIG. 26. Summary of preferred procedure to select affinity regions for protein misfolding-disease specific diagnostics and therapeutics.

Affinity regions directed against any set of misfolded proteins can be selected by applying a composition comprising affinity regions on an affinity matrix of misfolded proteins. When such matrix contains one or a set of misfolded proteins (mix X, column I) affinity regions preparation 1) are obtained that are directed against misfolded proteins in general. Such affinity regions can be applied for all misfolding diseases, but may cause side effects, since they are not all disease specific. Disease-specific affinity regions can be isolated by applying a composition of affinity regions on a column with one or a set of disease-specific misfolded proteins (mix A, column II). Affinity regions (preparation 2) obtained in such way contain disease-specific affinity regions, but also affinity regions that interact with misfolded proteins in general. The latter, similar to the affinity regions obtained from column I, may still cause side effects when applied for the specific disease, due to the presence of affinity regions that can bind to any misfolded protein that is present by occasion. Thus, more preferably, affinity regions (preparation 3) are prepared by applying a composition comprising affinity regions on a column of misfolded proteins (column I) and subsequently on a column with one or a set of disease-specific misfolded proteins (column III, similar or identical to column II). Even more preferably, affinity regions highly specific for misfolded proteins that contribute to the pathology of a disease (preparation 4) are obtained when a composition comprising affinity regions is applied subsequently on a column with one or a set of disease-specific misfolded proteins (column II) followed by a column (column IV) comprising any set of misfolded proteins but excluding those misfolded proteins that contribute to the pathology of the target disease and that are immobilized on column II, used to deplete the mixture of affinity regions collected with column II from those that generally interact with misfolded proteins.

### REFERENCES

Bouma, B. et al. Glycation induces formation of amyloid cross-β structure in albumin. J. Biol. Chem. 278, 41810-41819 (2003)
Citterio, S. et al. Dendritic cells as natural adjuvants. Methods 19, 142-147 (1999)
Gebbink, M. F., Claessen, D., Bouma, B., Dijkhuizen, L. & Wosten, H. A. 2005. Amyloids—a functional coat for microorganisms. Nat. Rev. Microbiol. 3, 333-341
Horbach, D. A., van Oort, E., Donders, R. C., Derksen, R. H. & de Groot, P. G. Lupus anticoagulant is the strongest risk factor for both venous and arterial thrombosis in patients with systemic lupus erythematosus. Comparison between different assays for the detection of antiphospholipid antibodies. Thromb. Haemost. 76, 916-924 (1996)
Kayed, R., E. Head, J. L. Thompson, T. M. Mclntire, S. C. Milton, C. W. Cotman, and C. G. Glabe. 2003. Common structure of soluble amyloid oligomers implies common mechanism of pathogenesis. Science 300:486-489 Common structure of soluble amyloid oligomers implies common mechanism of pathogenesis. Science 300: 486-489
Kranenburg, O., B. Bouma, L. M. Kroon-Batenburg, A. Reijerkerk, Y. P. Wu, E. E. Voest, and M. F. Gebbink. 2002. Tissue-type plasminogen activator is a multiligand cross-beta structure receptor. Curr. Biol. 12:1833-1839
O'Nuallain, B. & Wetzel, R. Conformational Abs recognizing a generic amyloid fibril epitope. Proc. Natl. Acad. Sci. U.S. A 99, 1485-1490 (2002)
Hackeng, T. M., Rosing, J., Spronk, H. M., and Vermeer, C. (2001) Protein Sci. 10, 864-870
Hackeng, T. M., Mounier, C. M., Bon, C., Dawson, P. E., Griffin, J. H., and Kent, S. B. (1997) Proc. Natl. Acad. Sci. U.S.A 94, 7845-7850
Rappsilber, J., Ishihama, Y., and Mann, M. (2003) Anal. Chem. 75, 663-670
Sallusto, F. and Lanzavecchia, A. (1994) J. Exp. Med. 179, 1109-1118
Winter, G., Griffiths, A. D., Hawkins, R. E., and Hoogenboom, H. R. (1994) Annu. Rev. Immunol. 12:433-55., 433-455
Hoogenboom, H. R. and Winter, G. (1992) J. Mol. Biol. %20; 227, 381-388
Hoogenboom, H. R. (2002) Methods Mol. Biol. 178:1-37., 1-37
Hoogenboom, H. R. and Chames, P. (2000) Immunol. Today. 21, 371-378
Hoogenboom, H. R. (1997) Trends Biotechnol. 15, 62-70
Hoogenboom, H. R. (2005) Nat. Biotechnol. 23, 1105-1116
de Kruif, J., Terstappen, L., Boel, E., and Logtenberg, T. (1995) Proc. Natl. Acad. Sci. U.S.A. 92, 3938-3942
de Kruif, J., Boel, E., and Logtenberg, T. (1995) J. Mol. Biol. 248, 97-105
Bloemendal, H. J., de Boer, H. C., Koop, E. A., van Dongen, A. J., Goldschmeding, R., Landman, W. J., Logtenberg, T., Gebbink, M. F., and Voest, E. E. (2004) Cancer Immunol. Immunother. 53, 799-808
Huls, G., Heijnen, I. A., Cuomo, E., van der, L. J., Boel, E., Van De Winkel, J. G., and Logtenberg, T. (1999) Cancer Res. 59, 5778-5784
Huls, G. A., Heijnen, I. A., Cuomo, M. E., Koningsberger, J. C., Wiegman, L., Boel, E., van der Vuurst de Vries A R, Loyson, S. A., Helfrich, W., Berge Henegouwen, G. P., van Meijer, M., de Kruif, J., and Logtenberg, T. (1999) Nat. Biotechnol. 17, 276-281
Boel, E., Verlaan, S., Poppelier, M. J., Westerdaal, N. A., van Strijp, J. A., and Logtenberg, T. (2000) J. Immunol. Methods. 239, 153-166
Morrison, S. L., Johnson, M. J., Herzenberg, L. A., and Oi, V. T. (1984) Proc. Natl. Acad. Sci. U.S.A. 81, 6851-6855

1. A method for selecting from a collection of IgIV molecules, at least one IgIV molecule comprising an affinity region that is capable of interacting with an epitope of a misfolded protein and/or with an epitope of a cross-β structure and/or with an epitope of a protein comprising a cross-β structure, said method comprising contacting a collection of IgIV molecules with a misfolded protein, a cross-β structure and/or a protein comprising a cross-β structure and collecting at least one IgIV molecule comprising an affinity region interacting with said epitope.

2. The method according to claim 1, wherein said epitope is at least part of a cross-β structure of a protein.

3. The method according to claim 1, wherein said epitope is exposed on said protein comprising a cross-β structure.

4. The method according to claim 1, wherein said misfolded protein, cross-β structure and/or protein comprising a cross-β structure is bound to a solid support.

5. A collection of IgIV molecules, enriched in IgIV molecules comprising an affinity region that is capable of interacting with an epitope of a misfolded protein, a cross-β structure and/or with an epitope of a protein comprising a cross-β structure.

6. The collection of IgIV molecules according to claim 5, selected by a method comprising:
contacting a collection of IgIV molecules with a misfolded protein, a cross-β structure, a protein comprising a cross-β structure, or a combination of any thereof, and
then selecting from the collection at least one IgIV molecule comprising an affinity region that interacts with said epitope.

7. A composition comprising at least 5 isolated, synthetic and/or recombinant molecules comprising an affinity region that is capable of interacting with an epitope of a misfolded protein, a cross-β structure and/or with an epitope of a protein comprising a cross-β structure.

8. The composition according to claim 7, comprising a functional part, derivative and/or analogue of at least one IgIV molecule comprising an affinity region capable of interacting with an epitope of a misfolded protein, a cross-β structure and/or with an epitope of a protein comprising a cross-β structure.

9. The composition according to claim 7, wherein at least one of said isolated, synthetic and/or recombinant molecules further comprises a cross-β structure binding molecule.

10. The composition according to claim 7, wherein at least one of said isolated, synthetic and/or recombinant molecules further comprises an effector molecule.

11. The composition according to claim 10, wherein said effector molecule is a protease or a cross-β structure-binding part thereof.

12. The composition according to claim 10, wherein said effector molecule is an immunopotentiating compound.

13. The composition according to claim 10, wherein said effector molecule is a complement activating factor.

14. The composition according to claim 10, wherein said effector molecule is a clearance signal.

15. The composition according to claim 10, wherein said effector molecule is an inflammation suppressive compound.

16. The composition according to claim 10, wherein said effector molecule is a cross-β structure binding-potentiating factor.

17. The composition according to claim 10, wherein said effector molecule is an opsonizing compound.

18. The composition according to claim 7, wherein said isolated, synthetic and/or recombinant molecule is an opsonizing compound.

19. A method for producing the composition of claim 7, the method comprising:
defining the amino acid sequence of an affinity region of at least one IgIV molecule capable of interacting with an epitope of a misfolded protein, a cross-β structure and/or with an epitope of a protein comprising a cross-β structure, and
producing isolated, synthetic and/or recombinant molecules comprising said amino acid sequence.

20. A method for selecting from the collection of IgIV molecules according to claim 5, a molecule comprising an affinity region which is capable, upon interacting with an epitope of a misfolded protein or a cross-β structure and/or upon interacting with an epitope of a protein comprising a cross-β structure, of inducing opsonization of said cross-β structure and/or protein by a phagocytic cell, said method comprising:
contacting the collection of IgIV molecules with a misfolded protein, a cross-β structure and/or with a protein comprising a cross-β structure;
contacting any complex comprising a misfolded protein, a cross-β structure and/or a protein comprising a cross-β structure, bound to an IgIV molecule and/or to an isolated, synthetic and/or recombinant molecule, with a phagocytic cell; and
collecting an IgIV molecule and/or isolated, synthetic and/or recombinant molecule that is capable of inducing or enhancing phagocytosis, by a phagocytic cell, of said misfolded protein, cross-β structure and/or protein comprising a cross-β structure.

21.-23. (canceled)

24. A method for increasing extracellular protein degradation and/or protein clearance in an individual, comprising administering the collection of IgIV molecules according to claim 5 to said individual.

25. A method for at least in part inhibiting misfolded protein and/or cross-β structure mediated effects in an individual, the method comprising:
administering an effective amount of claim 7 to an individual.

26. A method for at least partial prevention and/or treatment of a misfolded protein and/or cross β structure related and/or associated disease, a blood coagulation disorder, sepsis and/or a microbial/pathogen/bacterial/parasite/viral infection in an individual, the method comprising:
administering of claim 7 to the individual.

27. (canceled)

28. A composition comprising:
the composition of claim 7, and
a suitable carrier, diluent and/or excipient.

29. The composition according to claim 28, further comprising a cross-β structure-binding compound.

30. (canceled)

31. The composition of claim 28, further comprising a complement activating compound.

32. The composition of claim 28, further comprising an immunopotentiating compound, an inflammation suppressive compound, and/or a complement inhibiting compound.

33. (canceled)

34. (canceled)

35. A method for at least partially removing misfolded proteins, cross-β structures and/or proteins comprising a cross-β structure from a sample, said method comprising:
contacting a sample with a collection of IgIV molecules according to claim 5, and
removing from said sample a complex of a misfolded protein, and/or a cross-β structure, and/or protein comprising a cross-β structure, bound to an IgIV molecule and/or an isolated, synthetic and/or recombinant molecule.

36. The method according to claim 35, wherein said sample is a fluid sample comprising a body fluid together with a pharmaceutical constituent or food substance.

37.-40. (canceled)

41. A diagnostic kit comprising:
at least one affinity region of the collection of IgIV molecules according to claim 5, capable of interacting with a misfolded protein, with a cross-β structure and/or with a protein comprising a cross-β structure, and
a way of visualization of an interaction of said misfolded protein and/or said cross-β structure and/or said protein with said affinity region.

42. (canceled)

43. A method for determining whether a misfolded protein, and/or a protein and/or peptide comprising a cross-β structure is present in an aqueous solution comprising a protein, said method comprising:
contacting said aqueous solution with the collection of IgIV molecules according to claim 5, and
detecting whether bound misfolded protein, and/or a protein and/or peptide comprising a cross-β structure is present.

44. The method according to claim 43, wherein said aqueous solution comprises a detergent, a food product, a food supplement, a cell culture medium, a commercially available protein solution used for research purposes, blood, a blood product, cerebrospinal fluid, synovial fluid, lymph fluid, a cosmetic product, a cell, a pharmaceutical composition or any of its constituents comprising a protein, or a combination of any of these.

45. A method for removing a misfolded protein, a cross-β structure and/or a protein comprising a cross-β structure from a pharmaceutical composition or any of its constituents comprising a protein, said method comprising:
contacting said pharmaceutical composition or any of its constituents comprising a protein with the collection of IgIV molecules according to claim 5;
allowing binding of said misfolded protein, and/or protein and/or peptide comprising a cross-β structure to said collection of IgIV molecules and/or composition; and
separating bound protein and/or peptide comprising a cross-β structure from said pharmaceutical composition or any of its constituents comprising a protein.

46. The method according to claim 45, said method further comprising:
removing an unfolded protein, an unfolded peptide, a misfolded protein, a denatured protein, an aggregated protein, an aggregated peptide, a multimerized protein and/or a multimerized peptide, and/or a protein comprising a cross-β structure, from a pharmaceutical composition or any of its constituents so as to decrease and/or prevent undesired side effects of the pharmaceutical composition and/or increase the specific activity per gram protein of the pharmaceutical composition.

47. A pharmaceutical composition or any of its constituents comprising a protein, obtainable by a method according to claim 45.

48.-53. (canceled)

54. A method for interfering in coagulation of blood comprising providing to blood the composition of claim 7.

55. A method for determining the amount of misfolded proteins and/or cross-β structures in a composition, the method comprising:
contacting said composition with the collection of IgIV molecules according to claim 5, and
relating the amount of bound misfolded proteins and/or cross-β structures to the amount of cross-β structures present in said composition.

56. A method for determining a difference in cross-β structure content of a protein in a reference sample compared to cross-β structure content of said protein in a test sample, wherein said test sample has been subjected to a treatment that is expected to have an effect on the cross-β structure content of said protein, the method comprising:
determining in a reference sample the cross-β structure content of a protein using the collection of IgIV molecules according to claim 5;
subjecting said protein to a treatment that is expected to have an effect on the cross-β structure content of said protein, thus obtaining a test sample;
determining in the obtained test sample the cross-β structure content of said protein using the collection of IgIV molecules; and
determining whether the cross-β structure content of said protein in said reference sample is significantly different from the cross-β structure content of said protein in said test sample.

57.-66. (canceled)
